SEQUENCE LISTING (56 sequences)

SEQ ID NO: 1 - protein, 764 aa, Homo sapiens
MGSNLSPQLCLMPFILGLLSGGVTTTPWSLARPQGSCSLEGVEIKGGSFRLLQEGQALEYVCPSGFYPYPVQTRTCRSTGSWSTLKTQDQKTVRKAECRAIHCPRPHDFENGEYWPRSPYYNVSDEISFHCYDGYTLRGSANRTCQVNGRWSGQTAICDNGAGYCSNPGIPIGTRKVGSQYRLEDSVTYHCSRGLTLRGSQRRTCQEGGSWSGTEPSCQDSFMYDTPQEVAEAFLSSLTETIEGVDAEDGHGPGEQQKRKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 2 - protein, 6 aa, synthetic construct
  REGION 1..6: note = cleavage site
KVSEAD

SEQ ID NO: 3 - protein, 6 aa, synthetic construct
  REGION 1..6: note = cleavage site
GKKEAG

SEQ ID NO: 4 - protein, 6 aa, synthetic construct
  REGION 1..6: note = cleavage site
IRPSKG

SEQ ID NO: 5 - protein, 6 aa, synthetic construct
  REGION 1..6: note = cleavage site
GGEKRD

SEQ ID NO: 6 - protein, 226 aa, Homo sapiens
IIGGTECKPHSRPYMAYLEIVTSNGPSKFCGGFLIRRNFVLTAAHCAGRSITVTLGAHNITEEEDTWQKLEVIKQFRHPKYNTSTLHHDIMLLKLKEKASLTLAVGTLPFPSQFNFVPPGRMCRVAGWGRTGVLKPGSDTLQEVKLRLMDPQACSHFRDFDHNLQLCVGNPRKTKSAFKGDSGGPLLCAGVAQGIVSYGRSDAKPPAVFTRISHYRPWINQILQAN

SEQ ID NO: 7 - protein, 241 aa, Homo sapiens [sequence omitted]

SEQ ID NO: 8 - protein, 411 aa, Homo sapiens
SNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 9 - protein, 7 aa, synthetic construct
  REGION 1..7: note = cleavage site
QQKRKIV

SEQ ID NO: 10 - protein, 7 aa, synthetic construct
  REGION 1..7: note = cleavage site
WEHRKGT

SEQ ID NO: 11 - protein, 8 aa, synthetic construct
  REGION 1..8: note = cleavage site
KNQKRQKQ

SEQ ID NO: 12 - protein, 7 aa, synthetic construct
  REGION 1..7: note = cleavage site
DVFYQMI

SEQ ID NO: 13 - protein, 6 aa, synthetic construct
  REGION 1..6: note = cleavage site
EGVDAE

SEQ ID NO: 14 - protein, 7 aa, synthetic construct
  REGION 1..7: note = cleavage site
DHKLKSG

SEQ ID NO: 15 - protein, 6 aa, synthetic construct
  REGION 1..6: note = cleavage site
TPWSLA

SEQ ID NO: 16 - skipped

SEQ ID NO: 17 - protein, 9 aa, synthetic construct
  REGION 1..9: note = synthetic construct
  MOD_RES 1: note = Conjugated to CPQ2 quencher
  MOD_RES 7: note = Conjugated to 5FAM fluorescent reagent
  SITE 8..9: note = linked by PEG8 polymer of ethylene glycol
  MOD_RES 9: note = amidated
ITLLSAKKK

SEQ ID NO: 18 - protein, 855 aa, Homo sapiens [sequence omitted]

SEQ ID NO: 19 - protein, 263 aa, Homo sapiens
MAFLWLLSCWALLGTTFGCGVPAIHPVLSGLSRIVNGEDAVPGSWPWQVSLQDKTGFHFCGGSLISEDWVVTAAHCGVRTSDVVVAGEFDQGSDEENIQVLKIAKVFKNPKFSILTVNNDITLLKLATPARFSQTVSAVCLPSADDDFPAGTLCATTGWGKTKYNANKTPDKLQQAALPLLSNAECKKSWGRRITDVMICAGASGVSSCMGDSGGPLVCQKDGAWTLVGIVSWGSDTCSTSSPGVYARVTKLIPWVQKILAAN

SEQ ID NO: 20 - protein, 6 aa, synthetic construct
  REGION 1..6: note = cleavage site
KVSEAD

SEQ ID NO: 21 - protein, 7 aa, synthetic construct
  REGION 1..7: note = cleavage site
DHKLKSG

SEQ ID NO: 22 - protein, 252 aa, Homo sapiens
IIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLA

SEQ ID NO: 23 - protein, 227 aa, Homo sapiens
IINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 24 - protein, 246 aa, Homo sapiens
MRGSHHHHHHGSDYKDDDDKIIGGTECKPHSRPYMAYLEIVTSNGPSKFCGGFLIRRNFVLTAAHCAGRSITVTLGAHNITEEEDTWQKLEVIKQFRHPKYNTSTLHHDIMLLKLKEKASLTLAVGTLPFPSQFNFVPPGRMCRVAGWGRTGVLKPGSDTLQEVKLRLMDPQACSHFRDFDHNLQLCVGNPRKTKSAFKGDSGGPLLCAGVAQGIVSYGRSDAKPPAVFTRISHYRPWINQILQAN

SEQ ID NO: 25 - protein, 270 aa, Homo sapiens
MRGSHHHHHHGSDEKDSDSGLRSFTRQARVVGGTDADEGEWPWQVSLHALGQGHICGASLISPNWLVSAAHCYIDDRGFRYSDPTQWTAFLGLHDQSQRSAPGVQERRLKRIISHPFFNDFTFDYDIALLELEKPAEYSSMVRPICLPDASHVFPAGKAIWVTGWGHTQYGGTGALILQKGEIRVINQTTCENLLPQQITPRMMCVGFLSGGVDSCQGDSGGPLSSVEADGRIFQAGVVSWGDGCAQRNKPGVYTRLPLFRDWIKENTGV

SEQ ID NO: 26 - protein, 273 aa, Homo sapiens
MYRMQLLSCIALSLALVTNSIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 27 - protein, 293 aa, Homo sapiens
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 28 - protein, 271 aa, Homo sapiens
VTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 29 - protein, 10 aa, synthetic construct
  REGION 1..10: note = peptide substrate
  MOD_RES 1: note = Conjugated to TQ2 quencher
  MOD_RES 10: note = Conjugated to 5FAM fluorescent reagent
  MOD_RES 10: note = amidated
KDVFYQMKKK

SEQ ID NO: 30 - protein, 10 aa, synthetic construct
  REGION 1..10: note = peptide substrate
  MOD_RES 1: note = Conjugated to TQ2 quencher
  MOD_RES 10: note = Conjugated to 5FAM fluorescent reagent
KDVFYQMKKK

SEQ ID NO: 31 - protein, 10 aa, synthetic construct
  REGION 1..10: note = peptide substrate
  MOD_RES 1: note = Conjugated to 5FAM fluorescent reagent
  MOD_RES 10: note = Conjugated to QXL520 quencher
  MOD_RES 10: note = amidated
EQQKRKIVLK

SEQ ID NO: 32 - protein, 7 aa, synthetic construct
  REGION 1..7: note = peptide substrate
  MOD_RES 1: note = Conjugated to CPQ2 quencher
  MOD_RES 7: note = Conjugated to 5FAM fluorescent reagent
  MOD_RES 7: note = amidated
PEQQKRK

SEQ ID NO: 33 - protein, 11 aa, synthetic construct
  REGION 1..11: note = peptide substrate
  MOD_RES 1: note = Conjugated to TQ2 quencher
  MOD_RES 11: note = Conjugated to 5FAM fluorescent reagent
  MOD_RES 11: note = amidated
GEQQKRKIVLK

SEQ ID NO: 34 - protein, 4 aa, synthetic construct
  REGION 1..4: note = peptide substrate
  MOD_RES 1: note = acetylated
  MOD_RES 4: note = conjugated to 7-amino-4-carbamoylmethylcoumarin
QQKR

SEQ ID NO: 35 - protein, 4 aa, synthetic construct
  REGION 1..4: note = cleavage site
EGVD

SEQ ID NO: 36 - protein, 4 aa, synthetic construct
  REGION 1..4: note = cleavage site
QQKR

SEQ ID NO: 37 - protein, 4 aa, synthetic construct
  REGION 1..4: note = cleavage site
DVFY

SEQ ID NO: 38 - protein, 5 aa, synthetic construct
  REGION 1..5: note = cleavage site
KNQKR

SEQ ID NO: 39 - protein, 4 aa, synthetic construct
  REGION 1..4: note = peptide substrate
  MOD_RES 1: note = acetylated
  MOD_RES 4: note = conjugated to 7-amino-4-carbamoylmethylcoumarin
EGVD

SEQ ID NO: 40 - protein, 8 aa, synthetic construct
  REGION 1..8: note = peptide substrate
  MOD_RES 1: note = Conjugated to CPQ2 quencher
  MOD_RES 7: note = Conjugated to 5FAM fluorescent reagent
  MOD_RES 8: note = amidated
EGVDAEKK

SEQ ID NO: 41 - protein, 18 aa, synthetic construct
  REGION 1..18: note = peptide substrate
  MOD_RES 1: note = Conjugated to biotin
  MOD_RES 18: note = Conjugated to tetramethylrhodamine (TAMRA) azide
  MOD_RES 18: note = amidated
GGGLSSLTETIEGVDAEK

SEQ ID NO: 42 - protein, 9 aa, synthetic construct
  REGION 1..9: note = peptide substrate
  MOD_RES 1: note = Conjugated to CPQ2 quencher
  MOD_RES 7: note = Conjugated to 5FAM fluorescent reagent
  SITE 8..9: note = linked by PEG8 polymer of ethylene glycol
  MOD_RES 1: note = Conjugated to biotin
  MOD_RES 9: note = amidated
EGVDAEKKK

SEQ ID NO: 43 - protein, 4 aa, synthetic construct
  REGION 1..4: note = peptide substrate
  MOD_RES 1: note = acetylated
  SITE 4: note = conjugated to 7-amino-4-carbamoylmethylcoumarin
WEHR

SEQ ID NO: 44 - protein, 4 aa, synthetic construct
  REGION 1..4: note = peptide substrate
  MOD_RES 1: note = acetylated
  MOD_RES 4: note = conjugated to 7-amino-4-carbamoylmethylcoumarin
  MOD_RES 4: note = conjugated to a trifluoroacetic acid salt
KNQK

SEQ ID NO: 45 - protein, 9 aa, synthetic construct
  REGION 1..9: note = peptide substrate
  MOD_RES 1: note = conjugated to a TQ2 quencher
  MOD_RES 9: note = conjugated to a 5FAM fluorescent reagent
  MOD_RES 9: note = amidated
KNQKRQKQK

SEQ ID NO: 46 - protein, 4 aa, synthetic construct
  REGION 1..4: note = peptide substrate
  MOD_RES 1: note = acetylated
  MOD_RES 4: note = conjugated to 7-amino-4-carbamoylmethylcoumarin
KNQK

SEQ ID NO: 47 - protein, 17 aa, synthetic construct
  REGION 1..17: note = peptide substrate
  MOD_RES 17: note = conjugated to a 5FAM fluorescent reagent
  MOD_RES 17: note = amidated
WSHPQFEKKNQKRQKQK

SEQ ID NO: 48 - protein, 4 aa, synthetic construct
  REGION 1..4: note = peptide substrate
  MOD_RES 4: note = conjugated to 7-amino-4-carbamoylmethylcoumarin
QQKR

SEQ ID NO: 49 - protein, 4 aa, synthetic construct
  REGION 1..4: note = peptide substrate
  MOD_RES 4: note = conjugated to 7-amino-4-carbamoylmethylcoumarin
GLAR

SEQ ID NO: 50 - protein, 6 aa, synthetic construct
  REGION 1..6: note = peptide substrate
  MOD_RES 6: note = conjugated to 5-carboxy-fluorescein-L-Lysine
QQKRKI

SEQ ID NO: 51 - protein, 4 aa, synthetic construct
  REGION 1..4: note = peptide substrate
  MOD_RES 4: note = conjugated to 7-amino-4-carbamoylmethylcoumarin
QHRR

SEQ ID NO: 52 - protein, 6 aa, synthetic construct
  REGION 1..6: note = peptide substrate
  MOD_RES 6: note = conjugated to 5-carboxy-fluorescein-L-Lysine
EGVDAE

SEQ ID NO: 53 - protein, 6 aa, synthetic construct
  REGION 1..6: note = peptide substrate
EGVDAE

SEQ ID NO: 54 - protein, 4 aa, synthetic construct
  REGION 1..4: note = peptide substrate
  MOD_RES 4: note = conjugated to 7-amino-4-carbamoylmethylcoumarin
RQAR

SEQ ID NO: 55 - protein, 6 aa, synthetic construct
  REGION 1..6: note = peptide substrate
  MOD_RES 6: note = conjugated to 5-carboxy-fluorescein-L-Lysine
DVFYQM

SEQ ID NO: 56 - protein, 6 aa, synthetic construct
  REGION 1..6: note = peptide substrate
  MOD_RES 6: note = conjugated to 5-carboxy-fluorescein-L-Lysine
NVFYQM

(19) United States
(12) Patent Application Publication
(10) Pub. No.: US 2024/0117334 A1
(43) Pub. Date: Apr. 11, 2024

(54) FACTOR B PROTEASES

(71) Applicant: Vertex Pharmaceuticals Incorporated, Boston, MA (US)

(72) Inventors: Mikhail POPKOV, San Diego, CA (US); Vanessa SOROS, San Francisco, CA (US); Natacha LE MOAN, San Francisco, CA (US); Grant E. BLOUSE, Burlingame, CA (US); Timothy MYLES, South San Francisco, CA (US)

(21) Appl. No.: 18/219,384

(22) Filed: Jul. 7, 2023

Related U.S. Application Data
(63) Continuation of application No. PCT/US2022/011873, filed on Jan. 10, 2022.
(60) Provisional application No. 63/135,496, filed on Jan. 8, 2021.
(60) Provisional application No. 63/221,108, filed on Jul. 13, 2021.

(51) Int. Cl. C12N 9/72; A61P 37/06; C12N 9/64; C12N 9/76

(52) U.S. Cl. CPC C12N 9/6462; A61P 37/06; C12N 9/6424; C12N 9/6427; C12N 9/6445; C07K 2319/31; C12Y 304/21004; C12Y 304/21035; C12Y 304/21039; C12Y 304/21073

(57) ABSTRACT

Provided herein are engineered proteases of the S1A family that are specific for, and capable of, cleaving Factor B. Also provided herein are methods of making and using such engineered proteases. The engineered proteases provided herein may be useful for treating a disease or condition associated with dysregulation of the complement system by reducing complement activation through cleavage and inactivation of Factor B.

### CROSS REFERENCE TO RELATED APPLICATION

This application is a continuation of International PCT Patent Application No. PCT/US2022/011873, filed on Jan. 10, 2022, which claims priority to U.S. Provisional Application No. 63/135,496 filed on Jan. 8, 2021, and U.S. Provisional Application No. 63/221,108 filed on Jul. 13, 2021, the contents of each of which are incorporated herein by reference in their entireties.

### REFERENCE TO AN ELECTRONIC SEQUENCE LISTING

The contents of the electronic sequence listing (VTEX_704_02US_SeqList_ST26.xml; Size: 89,270 bytes; and Date of Creation: Dec. 11, 2023) are herein incorporated by reference in its entirety.

### BACKGROUND

The complement system includes the classical, alternative, and lectin pathways, and is tightly controlled by a number of regulators and components. One such component is Complement Factor B (interchangeably referred to herein as CFB, Factor B, FB), a serine protease proenzyme that circulates in blood as a single chain polypeptide. When Factor B associates with the active forms of C3, such as surface-bound C3b or fluid-phase C3(H₂O), to form a proconvertase complex, Factor B can subsequently be cleaved by Factor D into two fragments, Ba and Bb. The cleavage site of Factor B targeted by Factor D includes an Arg234-Lys235 bond. The resulting Ba cleavage product is non-catalytic and released from the complex, while the resulting Bb cleavage product is a catalytic serine protease that can then cleave C3 into C3a and C3b. This generation of C3b is part of an amplification loop of the complement system, allowing C3b to bind to another Factor B to form C3bBb.

Factor B and its cleavage products regulate complement activation. Dysregulated complement has been implicated in diseases involving the complement system, and thus needed are methods for modulating or inhibiting particular points of regulation within the complement system, such as the generation of the inactive Factor B fragments. Provided herein are compositions and methods that address these needs.

### SUMMARY

Provided herein are engineered, non-naturally occurring chymotrypsin-like serine proteases. Also provided herein are methods of making and using such non-naturally occurring chymotrypsin-like serine protease. The engineered proteases provided herein may be useful for treating a disease or condition associated with dysregulation of the complement system, or overactivation of complement.

Accordingly, in one aspect, provided herein is an engineered protease of the S1A serine protease family, wherein the engineered protease is specific for and is capable of cleaving Factor B. More specifically, the engineered protease of the disclosure comprises a modified chymase protease domain, a modified membrane type serine protease 1 (MTSP-1) protease domain, a modified urokinase-type plasminogen activator (uPA) protease domain, or a modified Kallikrein-related peptidase 5 (KLK5) protease domain, wherein the engineered protease is capable of cleaving Factor B. Modifications include one or more of a substitution, addition, and deletion of one or more amino acid residues, and/or one or more of a substitution, addition, and deletion of one or more domains of the chymotrypsin-like serine protease.

In some embodiments, the engineered protease is based on a MTSP-1 protease domain. In some embodiments, the engineered protease is not based on a MTSP-1 protease domain. In some embodiments, the engineered protease comprises one or more modifications with respect to a MTSP-1 protease domain comprising an amino acid sequence as set forth in SEQ ID NO: 7. In some embodiments, the one or more modifications are selected from those presented in Table 5A. In some embodiments, the one or more modifications are selected from those exemplary mutation strings presented in Table 5B. In some embodiments, the engineered protease comprises a modified membrane type serine protease 1 (MTSP-1) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 7.

In some embodiments, the engineered protease is based on a uPA protease domain. In some embodiments, the engineered protease is not based on a uPA protease domain. In some embodiments, the engineered protease comprises one or more modifications with respect to a uPA protease domain comprising an amino acid sequence as set forth in SEQ ID NO: 22. In some embodiments, the one or more modifications are selected from those presented in Table 3A. In some embodiments, the one or more modifications are selected from those exemplary mutation strings presented in Table 3B. In some embodiments, the engineered protease comprises a modified urokinase-type plasminogen activator (uPA) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 22.

In some embodiments, the engineered protease comprises one or more modifications with respect to a chymase protease domain comprising an amino acid sequence as set forth in SEQ ID NO: 6. In some embodiments, the one or more modifications are selected from those presented in Table 7A. In some embodiments, the one or more modifications are selected from those exemplary mutation strings presented in Table 7B. In some embodiments, the engineered protease comprises a modified chymase protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 6.

In some embodiments, the engineered protease is based on KLK5 protease domain, optionally comprising one or more amino acid modifications of SEQ ID NO: 23. In some embodiments, the engineered protease comprises a modified Kallikrein-related peptidase 5 (KLK5) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 23.

In some embodiments, cleavage of Factor B by the engineered protease generates one or more functionally inactive fragments. In some embodiments, the one or more functionally inactive fragments are capable of reducing complement activation. In some embodiments, cleavage of Factor B results in the generation of a Factor B fragment that is reduced in function.

In some embodiments, the Factor B is a rodent Factor B. In some embodiments, the Factor B is a non-human primate Factor B. In some embodiments, the non-human primate is cynomolgus monkey. In some embodiments, the Factor B is human Factor B. In some embodiments, the Factor B comprises the amino acid sequence as set forth in SEQ ID NO: 1.

In some embodiments, cleavage of Factor B occurs at a site not targeted by Factor D. In some embodiments, cleavage at the site generates at least two fragments that are not Ba and Bb. In some embodiments, cleavage at the site results in a reduction of the generation of Factor B cleavage products Ba and Bb as compared to cleavage by Factor D.

In some embodiments, cleavage of Factor B occurs at a site that is targeted by Factor D. In some embodiments, the site targeted by Factor D comprises QQKR/KIV (SEQ ID NO: 9). In some embodiments, the Factor B cleavage site comprises a sequence selected from: WEHR/KGT (SEQ ID NO: 10), KNQKR/QKQ (SEQ ID NO: 11), DVFY/QMI (SEQ ID NO: 12), EGVD/AE (SEQ ID NO: 13), DHKL/KSG (SEQ ID NO: 14), TPW/SLA (SEQ ID NO: 15), KVSEAD (SEQ ID NO: 20), IRPSKG (SEQ ID NO: 4), GGEKRD (SEQ ID NO: 5), GKKEAG (SEQ ID NO: 3), and DHKL/KSG (SEQ ID NO: 21).

In some embodiments, the engineered protease is based on MTSP-1 or uPA (includes a MTSP-1 or uPA protease domain) and the cleavage site in the Factor B comprises a sequence selected from: WEHR/KGT (SEQ ID NO: 10) and KNQKR/QKQ (SEQ ID NO: 11).

In some embodiments, the engineered protease is based on chymase protease domain. In some embodiments, the engineered protease is based on chymase protease domain, and the cleavage site comprises a sequence selected from DVFY/QMI (SEQ ID NO: 12), EGVD/AE (SEQ ID NO: 13), DHKL/KSG (SEQ ID NO: 14), and TPW/SLA (SEQ ID NO: 15).

In some embodiments, cleavage of Factor B results in the generation of a Factor B fragment that is reduced in function or results in a Factor B that is reduced in function. In some embodiments, the function of Factor B or a Factor B fragment is an interaction with at least one complement component. In some embodiments, the function of Factor B or a Factor B fragment is an interaction with hydrolyzed soluble C3. In some embodiments, the function of Factor B or a Factor B fragment is an interaction with C3b. In some embodiments, the function of Factor B or a Factor B fragment is an interaction with membrane-bound C3b.

In some embodiments, cleavage occurs when Factor B is not bound to C3b.

In some embodiments, the cleavage activity for a non-Factor B peptide substrate is about equal to or less than cleavage activity for the Factor B site.

In some embodiments, the engineered protease has a k_{cat}/Kₘ of about 100, about 200, about 300, about 400, about 500, about 600, about 700, about 800, about 900, about 1,000, about 1,000, about 1,100, about 1,200, about 1,300, about 1,400, about 1,500, about 1,600, about 1.700, about 1,800, or about 1,900 M⁻¹ s⁻¹ for Factor B cleavage. In some embodiments, the engineered protease has a k_{cat}/Kₘ of about 10³ to about 10⁹ M⁻¹ s⁻¹ for Factor B cleavage. In some embodiments, the engineered protease has an EC₅₀ for Factor B of less than about 20 nM. In some embodiments, the engineered protease has an EC₅₀ for Factor B of less than about 1 nM. In some embodiments, the engineered protease has an EC₅₀ for Factor B of about 20, about 25, or about 60 nM. In some embodiments, the engineered protease has an EC₅₀ for cleaving Factor B of about 1,000 to about 4,500 nM.

In some embodiments, the engineered protease has a plasma half-life in human plasma of over about 72 hours. In some embodiments, the engineered protease has a plasma half-life in human plasma of over about 120 hours. In some embodiments, the engineered protease has a plasma half-life in human plasma of about 7 days. In some embodiments, the catalytic activity is about 10% to about 50%, or about 90% to about 100%.

In some embodiments, the engineered protease has an increased half-life compared to an MTSP-1 protease domain that is not modified. In some embodiments, the engineered protease has an increased bioavailability compared to an MTSP-1 protease domain that is not modified. In some embodiments, the engineered protease has an increased half-life compared to a uPA protease domain that is not modified. In some embodiments, the engineered protease has an increased bioavailability compared to a uPA protease domain that is not modified. In some embodiments, the engineered protease has an increased half-life compared to a chymase that is not modified. In some embodiments, the engineered protease has an increased bioavailability compared to a chymase protease domain that is not modified. In some embodiments, the engineered protease has an increased bioavailability compared to a KLK5 protease domain that is not modified.

In some embodiments, the engineered protease is non-immunogenic.

In some embodiments, the engineered protease is in a zymogen form. In some embodiments, the engineered protease is in an active form.

In some embodiments, the engineered protease further comprises a half-life extender. Exemplary half-life extenders include Human Serum Albumin (HSA) and Fc (e.g., IgG1) fused to the engineered protease.

In another aspect, a method of inactivating Factor B is provided, comprising contacting the Factor B with any of the engineered proteases of the disclosure. In some embodiments, complement activation is inhibited. In some embodiments, the classical pathway of the complement pathway is inhibited. In some embodiments, the alternate pathway of the complement pathway is inhibited. In some embodiments, the lectin pathway of the complement pathway is inhibited.

In some embodiments, the method is in vitro. In some embodiments, the method is in vivo.

In another aspect, a method of treating a disease or condition in a subject in need thereof is provided, comprising administering to the subject any one of the engineered proteases of the disclosure. In some embodiments, the disease or condition is associated with dysregulated complement. In some embodiments, the disease or condition is an inflammatory disease or condition. In some embodiments, the treatment is a replacement therapy. In some embodiments, the treatment blocks complement activation. In some embodiments, the treatment modulates autoimmunity. In some embodiments, the disease or condition is a congenital complement deficiency. In some embodiments, the treatment is for endothelial or kidney cell injury.

In some embodiments, the disease or condition is selected from lupus nephritis, C3 glomerulopathy (C3G), primary IgA nephropathy, kidney transplant ischemia and reperfusion (I/R) injury, antineutrophil cytoplasmic antibody (ANCA)-associated vasculitides (AAV), atypical hemolytic uremic syndrome (aHUS), membranous nephropathy (MN) and paroxysmal nocturnal hemoglobinuria (PNH). In some embodiments, the disease or condition is a control protein deficiency. In some embodiments, the disease or condition is a secondary complement disorder. In some embodiments, the disease or condition is an immunity-related disease or condition.

In some embodiments, the engineered protease is administered to the subject subcutaneously.

In some embodiments, engineered protease is activated in situ at the site of a dysregulated complement component.

In some embodiments, the engineered protease is provided in a liquid stable formulation.

In another aspect, a pharmaceutical composition comprising any of the engineered proteases of the disclosure, and optionally a pharmaceutically acceptable carrier, is provided.

In some embodiments the engineered protease is provided in a liquid stable formulation.

In some embodiments the composition is formulated for subcutaneous administration.

### BRIEF DESCRIPTION OF THE DRAWINGS

FIG. 1A depicts a schematic diagram of a naturally occurring Factor B, showing the site at which Factor B is cleaved by Factor D (FD cleavage site; SEQ ID NOs: 13, 9, 20, residues 1-6 of SEQ ID NO: 12, SEQ ID NOs: 4, 5, 3, and residues 1-6 of SEQ ID NO: 11).

FIG. 1B and FIG. 1C depict two views of the protein structure of Factor B showing various cleavage sites (SEQ ID NOs: 13, 9, 11, 10, residues 1-6 of SEQ ID NO: 12 and SEQ ID NO: 14).

FIG. 1D depicts a Coomassie gel showing examples of Factor B cleavage by chymase-based engineered proteases.

FIG. 1E depicts a graph showing examples of Factor B cleavage by two chymase-based engineered proteases having low EC₅₀.

FIG. 2A and FIG. 2B depict schematic diagrams of a pro-chymase and a mature chymase, respectively. The mature chymase provides an exemplary scaffold of the disclosure.

FIG. 3A and FIG. 3B depict schematic diagrams of the extracellular portion of Membrane Type Serine Protease 1 (MTSP-1) and the serine protease domain of MTSP-1, respectively.

FIG. 3C and FIG. 3D depict schematic diagrams of urokinase-type plasminogen activator (uPA or u-PA).

FIG. 4A, FIG. 4B, FIG. 4C, FIG. 4D, and FIG. 4E depict graphs showing the stability of the engineered chymase-based engineered proteases tested using a peptide substrate.

FIG. 5 depicts a bar graph of controls used for a Factor B add-back hemolysis assay.

FIG. 6 depicts a graph showing the standard curves of the results of the Factor B add-back hemolysis assay.

FIG. 7A depicts a graph showing hemolysis inhibition with KLK5 and a chymase-based engineered protease C22S/F173Y/D175N/A226R.

FIG. 7B depicts a graph showing FB cleavage with two concentrations of KLK5 and a chymase-based engineered protease C22S/F173Y/D175N/A226R.

FIG. 8A and FIG. 8B depict Coomassie gels showing examples of Factor B cleavage with KLK5 and a chymase-based engineered protease C22S/F173Y/D175N/A226R.

FIG. 9 depicts mass spectrometry (MS) data identifying the cleavage site at 234 Arg within the QQKR/KIV (SEQ ID NO: 9) cleavage site of Factor B by KLK5, and identifying the cleavage site at 221 Asp within the EGVDAE (SEQ ID NO: 13) cleavage site of Factor B by the chymase-based engineered protease C22S/A226R.

FIG. 10 is a schematic depicting the general method for measuring complement activation and cytokine release from tissues of a mouse model of acute respiratory distress syndrome (ARDS) treated with a chymase-based engineered protease.

FIG. 11A, FIG. 11B, and FIG. 11C depict the pulmonary congestion index, body weight loss, and neutrophil to lymphocyte ratio in bronchoalveolar lavage fluid (BALF NLR), respectively, measured in a mouse model of acute respiratory distress syndrome (ARDS).

FIG. 12A, FIG. 12B, FIG. 12C, and FIG. 12D depict the results of BALF and lung cytokine measurement from mouse tissue after treatment with a chymase-based engineered protease.

FIG. 13 is a schematic depicting the general method for measuring lung function in a mouse model of acute respiratory distress syndrome (ARDS) treated with a chymase-based engineered protease of the disclosure.

FIG. 14 depicts the results of plethysmography measurements and significant protection to pulmonary congestion upon administration of one of the chymase-based engineered proteases of the disclosure.

FIG. 15 is a SDS-PAGE (reduced) depicting the expression, purification, and activation of a chymase-based engineered protease of the disclosure.

FIG. 16 is a SDS-PAGE depicting expression of chymase-based engineered proteases in HEK293 cells.

FIG. 17 is a SDS-PAGE depicting expression of chymase-based engineered proteases fused to human HSA or Fc in HEK293 cells.

### DETAILED DESCRIPTION

The disclosure provides compositions and methods useful for modulating the signaling and regulation of the complement system. Specifically, provided herein are engineered proteases comprising protease domains of the chymotrypsin-like S1A serine protease family such engineered proteases of the disclosure are specific for, and capable of cleaving Factor B. These engineered proteases comprise modified protease domains and are generated using the methods and sequences provided herein.

The engineered proteases of the disclosure target Factor B for cleavage and are interchangeably referred to as “Complement Factor B degraders” or “CFB degraders”. Use of these engineered proteases may (1) result in the cleavage of Factor B into fragments that are neither Ba nor Bb, or may (2) result in the cleavage of Factor B into Ba and Bb but which are functionally inactive. Without being bound to any theory or mechanism, fragments Ba and Bb resulting from cleavage of Factor B while Factor B is not complexed with C3 or C3b, are thought to be functionally inactive fragments and may further reduce or inhibit complement activation, act to limit an increase complement activation, and/or limit/reduce the amplification of complement pathways. In either of these scenarios, these cleavage products may have a function that is not a native function of Ba and Bb, or may be inactive fragments. As used herein, a “functionally inactive” fragment of Factor B refers to a fragment of Factor B, which is a cleavage product that may reduce complement activation, may limit an increase in complement activation, and/or may limit/reduce the amplification of the complement pathways.

In some embodiments, the engineered proteases target Factor B for cleavage at a cleavage site that is not targeted by Factor D. In other embodiments, the engineered proteases target Factor B at a site that is targeted by Factor D, before Factor B associates with and forms a complex with C3b, thus preventing the formation of the proconvertase complex.

The disclosure also provides methods of making and using such engineered proteases, for example, in treating a disease or condition associated with complement dysregulation, e.g. treating an overactive complement response.

### Chymotrypsin-Like Serine Proteases Useful for Modulation of the Complement System by Targeting Factor B

Provided herein are engineered proteases comprising one or more modifications with respect to a naturally occurring chymotrypsin-like serine protease. As used herein, an “engineered” protease of the disclosure is a serine protease of the S1A family that is non-naturally occurring, and comprises one or more modifications with respect to a wild type or naturally occurring serine protease of the S1A family. As used herein, a “modification” to a naturally occurring chymotrypsin-like serine protease of the S1A family, includes one or more of: a deletion of one or more amino acid residues, a deletion of one or more domains, a substitution of one or more amino acid residues, an insertion of one or more amino acid residues, an insertion of one or more domains, and a substitution of one or more domains.

In some embodiments, the engineered proteases of the disclosure comprise a non-naturally occurring serine protease domain of the S1A family, which domain comprises one or more modifications with respect to a wild type or naturally occurring serine protease domain of the S1A family. As used herein, a “modification” to a naturally occurring chymotrypsin-like serine protease domain of the S1A family, includes one or more of a deletion of one or more amino acid residues, a substitution of one or more amino acid residues, and an insertion of one or more amino acid residues.

The engineered proteases of the disclosure comprise a non-naturally occurring serine protease domain of the S1A family. In some embodiments, the engineered proteases of the disclosure comprise a non-naturally occurring serine protease domain of the S1A family and also comprise additional sequences and/or additional domains. In some embodiments, the engineered proteases of the disclosure consist of a non-naturally occurring serine protease domain of the S1A family. In some embodiments, the engineered proteases of the disclosure consist essentially of a non-naturally occurring serine protease domain of the S1A family, and may include additional sequences useful for expression, stability, improved pharmacokinetics, subcutaneous delivery, tissue targeting and the like.

It is noted that as used herein, a “naturally occurring chymotrypsin-like serine protease” of the S1A family refers to such protease that is present in nature, even if it is not the wild type sequence. Stated differently, the naturally occurring serine protease is not engineered. The naturally occurring serine protease may be with or without a signal sequence, and with or without an activation peptide, and may be of any species.

The engineered proteases provided herein are designed to cleave Factor B. In some embodiments, the engineered proteases provided herein target Factor B at a non-Factor D cleavage site. In some embodiments, the engineered proteases provided herein target Factor B at a Factor D cleavage site. In some embodiments, the engineered proteases provided herein can target Factor B for cleavage while Factor B is in a complex with C3. In some embodiments, the engineered proteases provided herein can target Factor B for cleavage while Factor B in complex with C3b. In some embodiments, the engineered proteases provided herein can target Factor B for cleavage while Factor B is alone in circulation.

In some embodiments, cleavage products resulting from cleavage of Factor B by engineered proteases provided herein may be functionally inactive fragments, as discussed above. In some embodiments, the functionally inactive fragments do not have a naturally occurring physiological function. In some embodiments, the functionally inactive fragments may perform a function, but not necessarily the same function as Ba and/or Bb.

In some embodiments, the engineered proteases provided herein are capable of modulating the activity of the complement system by reducing the amount of Factor B fragments Ba and Bb that are generated, thereby dampening/inhibiting complement activation, limiting an increase complement activation, and/or limiting/reducing the amplification of complement pathways.

In some embodiments, the engineered proteases provided herein are useful for administration to a subject in need thereof. As used herein, the terms “patient” or “subject” are interchangeably used refer to mammals and include, without limitation, humans and other primates (e.g., chimpanzees, cynomolgus monkeys, and other apes and monkey species), farm animals (e.g., cattle, sheep, pigs, goats and horses), domestic mammals (e.g., dogs and cats), and laboratory animals (e.g., rabbits, rodents such as mice, rats, and guinea pigs). In exemplary embodiments, the subject is a human.

Table 1 provides the amino acid sequence of a human Factor B, targeted by the engineered proteases of the disclosure.

Table 1 also provides the amino acid sequences of exemplary chymotrypsin-like serine proteases and protease domains of the S1A family that can be utilized as scaffolds based upon which the engineered proteases of the disclosure are generated, including: MTSP-1, uPA, chymase, and Kallikrein-related peptidase 5 (KLK5). Accordingly, in some embodiments, the engineered proteases provided herein are based on MTSP-1, or the serine protease domain thereof. In some embodiments, the engineered proteases provided herein are based on uPA, or the serine protease domain thereof. In some embodiments, the engineered proteases provided herein are based on KLK5, or the serine protease domain thereof. In some embodiments, the engineered proteases provided herein not based on chymase or a serine protease domain thereof. In some embodiments, the engineered proteases provided herein are not based on MTSP-1 or a serine protease domain thereof. In some embodiments, the engineered proteases provided herein are not based on uPA or a serine protease domain thereof. In some embodiments, the engineered proteases provided herein are not based on KLK5 or a serine protease domain thereof. In some embodiments, the engineered proteases provided herein are not based on chymase or a serine protease domain thereof.

Table 1 also includes the amino acid sequence of a mature chymotrypsin polypeptide in SEQ ID NO: 19. It is noted that a protease domain of a serine protease can be aligned with that of chymotrypsin such that the amino acid residues of the aligned protease (e.g., MTSP-1, uPA, KLK5, or chymase) correspond to the amino acids of chymotrypsin which are then provided with the numbering of chymotrypsin. This is generally referred to herein as a chymotrypsin numbering, and the numbering, and corresponding positions of aligned proteases can be determined by one of skill in the art. Standard nomenclature useful for chymotrypsin numbering can also be determined by one of skill in the art, such as notations used for additions or deletions or residues. A residue existing in the aligned protease (e.g., MTSP-1, uPA, KLK5, or chymase) which does not exist in chymotrypsin, is provided with a lowercase letter notation. By way of example, using the chymotrypsin numbering key for the uPA protease domain (Table 2), the modification S37dP (chymotrypsin numbering) translates to S184P in conventional amino acid sequence notation. Signal or leader sequences are indicated by underlining, and cleavage site sequences are indicated by bold, in Table 1. In some instances, the disclosure and claims contain reference to conventional amino acid numbering, and/or chymotrypsin based numbering, and is so identified accordingly.

The chymase of Table 1 is a mast cell chymase, whose sequence can be found at https://www.uniprot.org/uniprot/P23946.

TABLE 1
Human Factor B and Wild Type Serine Protease Sequences

| | |
|---|---|
| Human Factor | MGSNLSPQLCLMPFILGLLSGGVTTTPWSLARPQGSCSLEGVEI |
| B | KGGSFRLLQEGQALEYVCPSGFYPYPVQTRTCRSTGSWSTLKT |
| | QDQKTVRKAECRAIHCPRPHDFENGEYWPRSPYYNVSDEISFH |
| | CYDGYTLRGSANRTCQVNGRWSGQTAICDNGAGYCSNPGIPI |
| | GTRKVGSQYRLEDSVTYHCSRGLTLRGSQRRTCQEGGSWSGT |
| | EPSCQDSFMYDTPQEVAEAFLSSLTETIEGVDAEDGHGPGEQQ |
| | KRKIVLDPSGSMNIYLVLDGSDSIGASNFTGAKKCLVNLIEKV |
| | ASYGVKPRYGLVTYATYPKIWVKVSEADSSNADWVTKQLNEI |
| | NYEDHKLKSGTNTKKALQAVYSMMSWPDDVPPEGWNRTRH |
| | VIILMTDGLHNMGGDPITVIDEIRDLLYIGKDRKNPREDYLDVY |
| | VFGVGPLVNQVNINALASKKDNEQHVFKVKDMENLEDVFYQ |
| | MIDESQSLSLCGMVWEHRKGTDYHKQPWQAKISVIRPSKGHE |
| | SCMGAVVSEYFVLTAAHCFTVDDKEHSIKVSVGGEKRDLEIE |
| | VVLFHPNYNINGKKEAGIPEFYDYDVALIKLKNKLKYGQTIRP |
| | ICLPCTEGTTRALRLPPTTTCQQQKEELLPAQDIKALFVSEEEK |
| | KLTRKEVYIKNGDKKGSCERDAQYAPGYDKVKDISEVVTPRF |
| | LCTGGVSPYADPNTCRGDSGGPLIVHKRSRFIQVGVISWGVVD |
| | VCKNQKRQKQVPAHARDFHINLFQVLPWLKEKLQDEDLGFL |
| | (SEQ ID NO: 1) |
| Human | MRGSHHHHHHGSDYKDDDDKIIGGTECKPHSRPYMAYLEIVT |
| Chymase | SNGPSKFCGGFLIRRNFVLTAAHCAGRSITVTLGAHNITEEEDT |
| (signal | WQKLEVIKQFRHPKYNTSTLHHDIMLLKLKEKASLTLAVGTLP |
| sequence, | FPSQFNFVPPGRMCRVAGWGRTGVLKPGSDTLQEVKLRLMDP |
| protease | QACSHFRDFDHNLQLCVGNPRKTKSAFKGDSGGPLLCAGVAQ |
| domain) | GIVSYGRSDAKPPAVFTRISHYRPWINQILQAN (SEQ ID NO: 24) |
| Human | IIGGTECKPHSRPYMAYLEIVTSNGPSKFCGGFLIRRNFVLTAA |
| Chymase | HCAGRSITVTLGAHNITEEEDTWQKLEVIKQFRHPKYNTSTLH |
| Protease | HDIMLLKLKEKASLTLAVGTLPFPSQFNFVPPGRMCRVAGWG |
| Domain) | RTGVLKPGSDTLQEVKLRLMDPQACSHFRDFDHNLQLCVGNP |
| | RKTKSAFKGDSGGPLLCAGVAQGIVSYGRSDAKPPAVFTRISH |
| | YRPWINQILQAN (SEQ ID NO: 6) |
| Human | MGSDRARKGGGGPKDFGAGLKYNSRHEKVNGLEEGVEFLPV |
| MTSP-1 full | NNVKKVEKHGPGRWVVLAAVLIGLLLVLLGIGFLVWHLQYR |
| length | DVRVQKVFNGYMRITNENFVDAYENSNSTEFVSLASKVKDAL |
| | KLLYSGVPFLGPYHKESAVTAFSEGSVIAYYWSEFSIPQHLVEE |
| | AERVMAEERVVMLPPRARSLKSFVVTSVVAFPTDSKTVQRTQ |
| | DNSCSFGLHARGVELMRFTTPGFPDSPYPAHARCQWALRGDA |
| | DSVLSLTFRSFDLASCDERGSDLVTVYNTLSPMEPHALVQLCG |
| | TYPPSYNLTFHSSQNVLLITLITNTERRHPGFEATFFQLPRMSSC |
| | GGRLRKAQGTFNSPYYPGHYPPNIDCTWNIEVPNNQHVKVRF |
| | KFFYLLEPGVPAGTCPKDYVEINGEKYCGERSQFVVTSNSNKI |
| | TVRFHSDQSYTDTGFLAEYLSYDSSDPCPGQFTCRTGRCIRKEL |
| | RCDGWADCTDHSDELNCSCDAGHQFTCKNKFCKPLFWVCDS |
| | VNDCGDNSDEQGCSCPAQTFRCSNGKCLSKSQQCNGKDDCG |
| | DGSDEASCPKVNVVTCTKHTYRCLNGLCLSKGNPECDGKEDC |
| | SDGSDEKDCDCGLRSFTRQARVVGGTDADEGEWPWQVSLHA |
| | LGQGHICGASLISPNWLVSAAHCYIDDRGFRYSDPTQWTAFLG |
| | LHDQSQRSAPGVQERRLKRIISHPFFNDFTFDYDIALLELEKPA |
| | EYSSMVRPICLPDASHVFPAGKAIWVTGWGHTQYGGTGALIL |
| | QKGEIRVINQTTCENLLPQQITPRMMCVGFLSGGVDSCQGDSG |
| | GPLSSVEADGRIFQAGVVSWGDGCAQRNKPGVYTRLPLFRDW |
| | IKENTGV (SEQ ID NO: 18) |
| Human | MRGSHHHHHHGSDEKDSDSGLRSFTRQARVVGGTDADEGEW |
| MTSP-1 | PWQVSLHALGQGHICGASLISPNWLVSAAHCYIDDRGFRYSDP |
| (signal | TQWTAFLGLHDQSQRSAPGVQERRLKRIISHPFFNDFTFDYDIA |
| sequence, | LLELEKPAEYSSMVRPICLPDASHVFPAGKAIWVTGWGHTQY |
| protease | GGTGALILQKGEIRVINQTTCENLLPQQITPRMMCVGFLSGGV |
| domain) | DSCQGDSGGPLSSVEADGRIFQAGVVSWGDGCAQRNKPGVYT |
| | RLPLFRDWIKENTGV (SEQ ID NO: 25) |
| Human | VVGGTDADEGEWPWQVSLHALGQGHICGASLISPNWLVSAA |
| MTSP-1 | HCYIDDRGFRYSDPTQWTAFLGLHDQSQRSAPGVQERRLKRII |
| Protease | SHPFFNDFTFDYDIALLELEKPAEYSSMVRPICLPDASHVFPAG |
| Domain | KAIWVTGWGHTQYGGTGALILQKGEIRVINQTTCENLLPQQIT |
| | PRMMCVGFLSGGVDSCQGDSGGPLSSVEADGRIFQAGVVSWG |
| | DGCAQRNKPGVYTRLPLFRDWIKENTGV (SEQ ID NO: 7) |
| Human uPA | SNELHQVPSNCDCLNGGTCVSNKYFSNIHWCNCPKKFGGQHC |
| full length | EIDKSKTCYEGNGHFYRGKASTDTMGRPCLPWNSATVLQQTY |
| | HAHRSDALQLGLGKHNYCRNPDNRRRPWCYVQVGLKPLVQE |
| | CMVHDCADGKKPSSPPEELKFQCGQKTLRPRFKIIGGEFTTIEN |
| | QPWFAAIYRRHRGGSVTYVCGGSLISPCWVISATHCFIDYPKK |
| | EDYIVYLGRSRLNSNTQGEMKFEVENLILHKDYSADTLAHHN |
| | DIALLKIRSKEGRCAQPSRTIQTICLPSMYNDPQFGTSCEITGFG |
| | KENSTDYLYPEQLKMTVVKLISHRECQQPHYYGSEVTTKMLC |
| | AADPQWKTDSCQGDSGGPLVCSLQGRMTLTGIVSWGRGCAL |
| | KDKPGVYTRVSHFLPWIRSHTKEENGLAL (SEQ ID NO: 8) |
| Human uPA | MYRMQLLSCIALSLALVTNSIIGGEFTTIENQPWFAAIYRRHRG |
| (signal | GSVTYVCGGSLISPCWVISATHCFIDYPKKEDYIVYLGRSRLNS |
| sequence, | NTQGEMKFEVENLILHKDYSADTLAHHNDIALLKIRSKEGRCA |
| protease | QPSRTIQTICLPSMYNDPQFGTSCEITGFGKENSTDYLYPEQLK |
| domain) | MTVVKLISHRECQQPHYYGSEVTTKMLCAADPQWKTDSCQG |
| | DSGGPLVCSLQGRMTLTGIVSWGRGCALKDKPGVYTRVSHFL |
| | PWIRSHTKEENGLAL (SEQ ID NO: 26) |
| Human uPA | IIGGEFTTIENQPWFAAIYRRHRGGSVTYVCGGSLISPCWVISAT |
| Protease | HCFIDYPKKEDYIVYLGRSRLNSNTQGEMKFEVENLILHKDYS |
| Domain | ADTLAHHNDIALLKIRSKEGRCAQPSRTIQTICLPSMYNDPQFG |
| | TSCEITGFGKENSTDYLYPEQLKMTVVKLISHRECQQPHYYGS |
| | EVTTKMLCAADPQWKTDSCQGDSGGPLVCSLQGRMTLTGIVS |
| | WGRGCALKDKPGVYTRVSHFLPWIRSHTKEENGLA (SEQ ID |
| | NO: 22) |
| Human | MATARPPWMWVLCALITALLLGVTEHVLANNDVSCDHPSNT |
| Kallikrein 5 | VPSGSNQDLGAGAGEDARSDDSSSRIINGSDCDMHTQPWQA |
| (KLK5) | ALLLRPNQLYCGAVLVHPQWLLTAAHCRKKVFRVRLGHYSLS |
| (signal | PVYESGQQMFQGVKSIPHPGYSHPGHSNDLMLIKLNRRIRPTK |
| sequence | DVRPINVSSHCPSAGTKCLVSGWGTTKSPQVHFPKVLQCLNIS |
| (underlined), | VLSQKRCEDAYPRQIDDTMFCAGDKAGRDSCQGDSGGPVVC |
| propeptide, | NGSLQGLVSWGDYPCARPNRPGVYTNLCKFTKWIQETIQANS |
| and protease | (SEQ ID NO: 27) |
| domain) | |
| Human | VTEHVLANNDVSCDHPSNTVPSGSNQDLGAGAGEDARSDDSS |
| Kallikrein 5 | SRIINGSDCDMHTQPWQA |
| (KLK5) | ALLLRPNQLYCGAVLVHPQWLLTAAHCRKKVFRVRLGHYSLS |
| (propeptide, | PVYESGQQMFQGVKSIPHPGYSHPGHSNDLMLIKLNRRIRPTK |
| and protease | DVRPINVSSHCPSAGTKCLVSGWGTTKSPQVHFPKVLQCLNIS |
| domain) | VLSQKRCEDAYPRQIDDTMFCAGDKAGRDSCQGDSGGPVVC |
| | NGSLQGLVSWGDYPCARPNRPGVYTNLCKFTKWIQETIQANS |
| | (SEQ ID NO: 28) |
| Human | IINGSDCDMHTQPWQA |
| Kallikrein 5 | ALLLRPNQLYCGAVLVHPQWLLTAAHCRKKVFRVRLGHYSLS |
| Protease | PVYESGQQMFQGVKSIPHPGYSHPGHSNDLMLIKLNRRIRPTK |
| Domain | DVRPINVSSHCPSAGTKCLVSGWGTTKSPQVHFPKVLQCLNIS |
| | VLSQKRCEDAYPRQIDDTMFCAGDKAGRDSCQGDSGGPVVC |
| | NGSLQGLVSWGDYPCARPNRPGVYTNLCKFTKWIQETIQANS |
| | (SEQ ID NO: 23) |
| Chymotrypsin | MAFLWLLSCWALLGTTFGCGVPAIHPVLSGLSRIVNGEDAVPG |
| | SWPWQVSLQDKTGFHFCGGSLISEDWVVTAAHCGVRTSDVV |
| | VAGEFDQGSDEENIQVLKIAKVFKNPKFSILTVNNDITLLKLAT |
| | PARFSQTVSAVCLPSADDDFPAGTLCATTGWGKTKYNANKTP |
| | DKLQQAALPLLSNAECKKSWGRRITDVMICAGASGVSSCMGD |
| | SGGPLVCQKDGAWTLVGIVSWGSDTCSTSSPGVYARVTKLIP |
| | WVQKILAAN (SEQ ID NO: 19) |

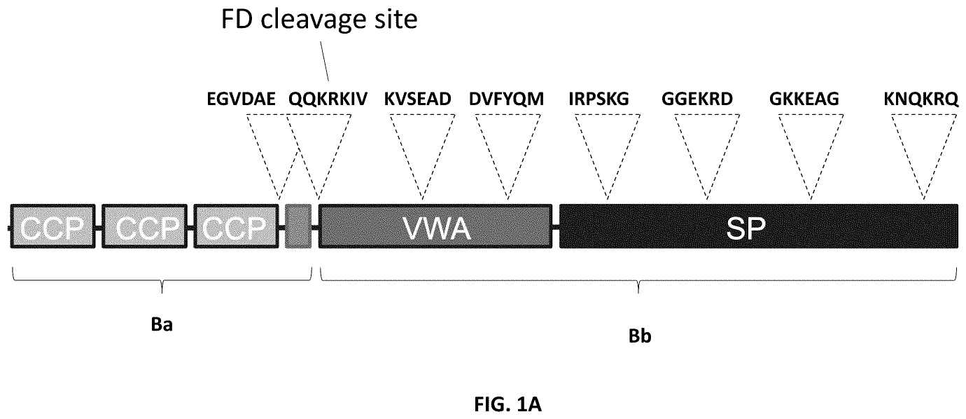

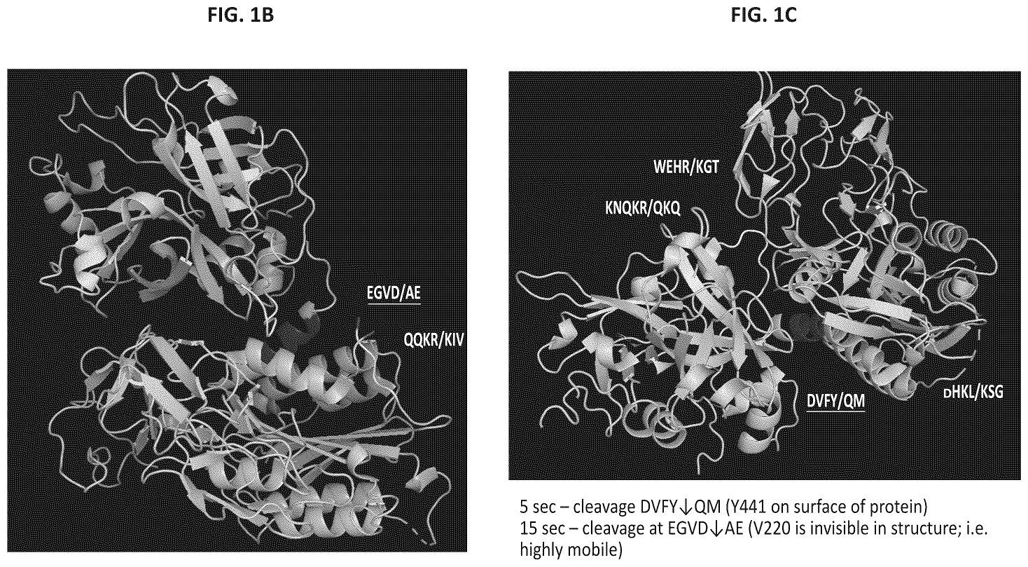

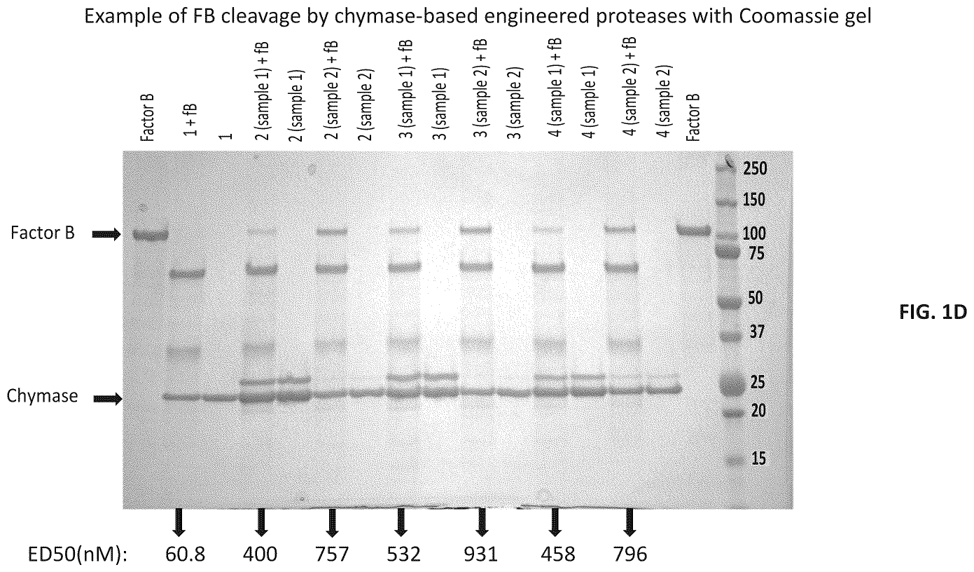

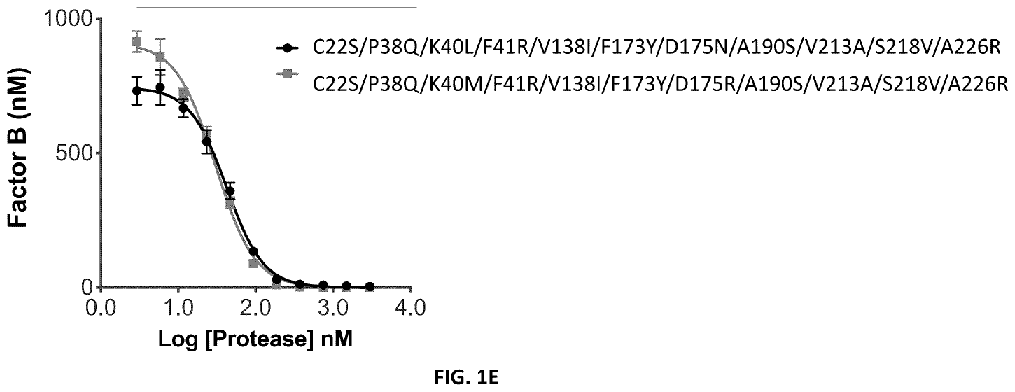

FIG. 1A depicts a schematic diagram of a naturally occurring Factor B, showing various cleavage site sequences, including the site at which Factor B is cleaved by Factor D (FD cleavage site) into Ba and Bb. Ba is made up of three complement control protein (CCP) domains, and a linker. Bb is made up of a von Willebrand Factor Type A (VWA) domain, and a serine protease (SP) domain. FIGS. 1B-1C depict two views of the protein structure of Factor B showing various cleavage sites.

In some embodiments, the disclosure provides engineered proteases that cleave Factor B, which can be at a cleavage site that is not targeted by Factor D, i.e., not at the FD cleavage site. In other embodiments, the disclosure provides engineered proteases that cleave Factor Bat a cleavage site that is targeted by Factor D. As contemplated herein, a FD cleavage site is a site that is the amino acid sequence of QQKR/KIV (SEQ ID NO: 9). In other embodiments, the disclosure also provides for engineered proteases that can cleave Factor B at a cleavage site that is targeted by Factor D (SEQ ID NO: 9), before Factor B forms a complex with C3b—without being bound by any theory or mechanism, it is expected that such cleavage can result in fragments that do not increase complement activity.

Various exemplary cleavage sites of Factor B that may be targeted by the engineered proteases of the disclosure are indicated on the schematic diagram of FIG. 1, and include, but are not limited to: QQKR/KIV (SEQ ID NO: 9), WEHR/KGT (SEQ ID NO: 10), KNQKR/QKQ (SEQ ID NO: 11), DVFY/QMI (SEQ ID NO: 12), EGVD/AE (SEQ ID NO: 13), DHKL/KSG (SEQ ID NO: 14), TPW/SLA (SEQ ID NO: 15), KVSEAD (SEQ ID NO: 20), IRPSKG (SEQ ID NO: 4), GGEKRD (SEQ ID NO: 5), GKKEAG (SEQ ID NO: 3), and DHKL/KSG (SEQ ID NO: 21). In some exemplary cleavage sites, a slash is used to represent the site of cleavage. However, the cleavage at these sites is not limited to such.

FIG. 1D depicts a Coomassie gel showing examples of Factor B cleavage by chymase-based engineered proteases. Two lots of each engineered protease set were used, and each lot showed the ability of the engineered proteases to cleave Factor B. FIG. 1E depicts a graph showing examples of Factor B cleavage by two chymase-based engineered proteases having low EC₅₀. These results are discussed in further detail in Example 1 below.

The amino acid sequence of a wild type human Factor B is presented in Table 1 below, shown by SEQ ID NO: 1. As depicted in Table 1, in some embodiments, a site comprising the amino acid sequence KVSEAD (SEQ ID NO: 2) may be targeted as a cleavage site by an engineered protease of the disclosure. In some embodiments an engineered protease that can target the sequence of SEQ ID NO: 2 for cleavage is based on chymase. In some embodiments, a site comprising the amino acid sequence GKKEAG (SEQ ID NO: 3) may be targeted as a cleavage site by an engineered protease of the disclosure. In some embodiments an engineered protease that can target the sequence of SEQ ID NO: 3 for cleavage is based on MTSP-1 or uPA. In some embodiments, a site comprising the amino acid sequences IRPSKG (SEQ ID NO: 4) and/or GGEKRD (SEQ ID NO: 5) may be targeted as a cleavage site by an engineered protease of the disclosure, in some embodiments such an engineered protease is built on a MTSP-1-based scaffold.

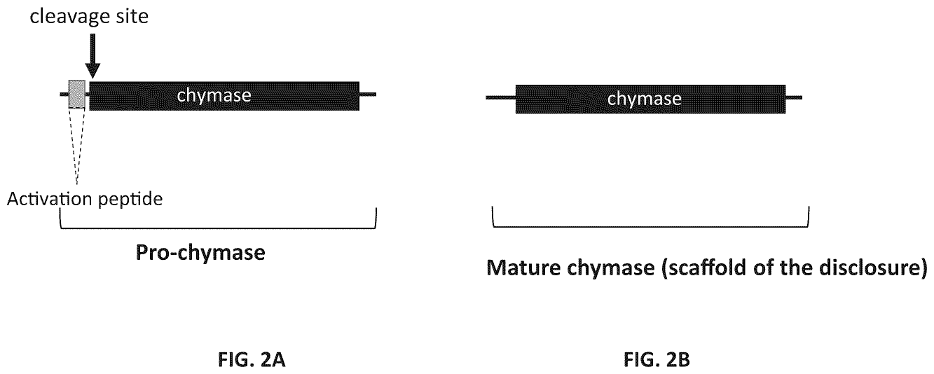

FIGS. 2A-2B depict schematic diagrams of a pro-chymase and a mature chymase, respectively. The pro-chymase comprises the chymase domain and an activation peptide, while the mature chymase is produced when the signal peptide and the activation peptide are cleaved at the cleavage site indicated in FIG. 2A. The mature chymase as depicted in FIG. 2B can be utilized as a scaffold for generation of engineered proteases of the disclosure. The amino acid sequence of a wild type mature chymase protease domain is presented in Table 1, shown by SEQ ID NO: 6.

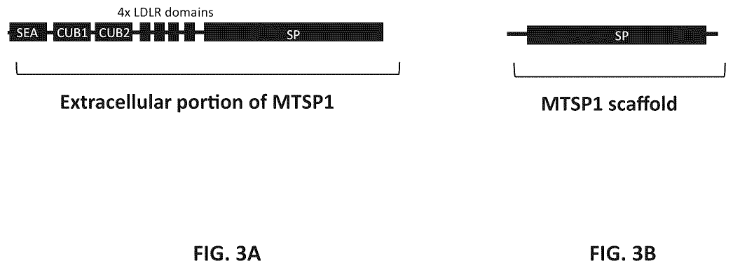

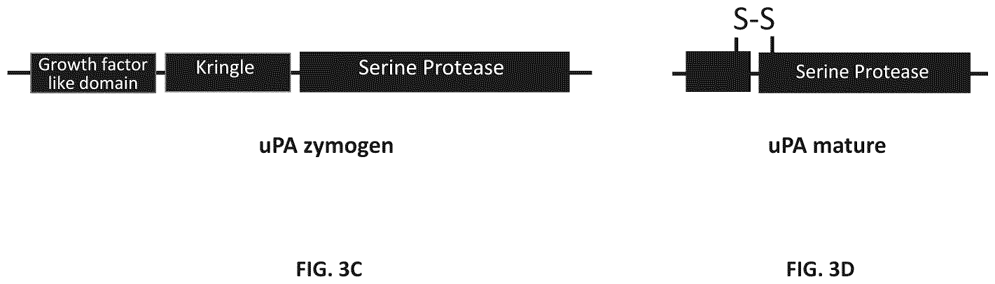

FIGS. 3A-3B depict schematic diagrams of the extracellular portion of Membrane Type Serine Protease 1 (MTSP-1) and the serine protease domain of MTSP-1, respectively. The MTSP-1 serine protease domain, as depicted in FIG. 3B, can be utilized as a scaffold for generation of engineered proteases of the disclosure. The amino acid sequence of the protease domain of a naturally occurring MTSP-1 is presented in SEQ ID NO: 7 in Table 1.

FIGS. 3C-3D depict schematic diagrams of urokinase-type plasminogen activator (uPA or u-PA). FIG. 3C depicts a schematic diagram of the uPA zymogen, and FIG. 3D depicts a schematic diagram of a mature two chain uPA. The mature uPA polypeptide is generated by proteolytic cleavage. The uPA serine protease domain, as depicted in FIG. 3D, can be utilized as a scaffold for generation of engineered proteases of the disclosure. In wild-type uPA the protease domain is connected to a “A” chain by a disulfide bridge (as depicted in FIG. 3D). In the engineered uPA-based proteases provided herein, comprising a uPA serine protease domain there can be a C to S substitution (C122S as presented by chymotrypsin numbering of SEQ ID NO: 22) to reduce aggregation.

In some embodiments, provided herein are engineered proteases, wherein the serine proteases are specific for Factor B at a site that is not targeted by Factor D. In other embodiments, provided herein are engineered proteases, wherein the serine proteases are specific for Factor B at a site that is targeted by Factor D. In some embodiments, cleavage of Factor B by the engineered proteases provided herein at the site targeted by Factor D, or at the site not targeted by Factor D, results in a reduction of complement activation. In some embodiments, cleavage at the site generates at least two functionally inactive fragments. In some embodiments, cleavage of Factor B by the engineered proteases provided herein at the site targeted by Factor D, or at the site not targeted by Factor D, results generates one or more functionally inactive fragments. In some embodiments, cleavage of Factor B by the engineered proteases provided herein at the site targeted by Factor D, or at the site not targeted by Factor D, results in a reduction of a function of Factor B. In some embodiments, cleavage of Factor B by the engineered proteases provided herein at the site targeted by Factor D, or at the site not targeted by Factor D, results in a reduction of Factor B cleavage products Ba and Bb.

In some embodiments, the Factor B targeted by the engineered proteases of the disclosure can be of any species. In some embodiments, the Factor B is a primate Factor B. In some embodiments, the Factor B is human Factor B. In some embodiments, the human Factor B comprises the amino acid sequence as set forth in SEQ ID NO: 1. In some embodiments, the primate Factor B is a non-human primate Factor B. In some embodiments, the non-human primate is cynomolgus monkey. In some embodiments, the Factor B is a rodent Factor B, e.g., Factor B of a rat, or mouse.

In some embodiments, the engineered proteases provided herein are specific for Factor B at a site that is not targeted by Factor D, wherein the site targeted by Factor D comprises the amino acid sequence QQKR/KIV (SEQ ID NO: 9). In some embodiments, the site on Factor B that is not targeted by Factor D comprises a sequence selected from the group consisting of WEHR/KGT (SEQ ID NO: 10), KNQKR/QKQ (SEQ ID NO: 11), DVFY/QMI (SEQ ID NO: 12), EGVD/AE (SEQ ID NO: 13), DHKL/KSG (SEQ ID NO: 14), TPW/SLA (SEQ ID NO: 15), KVSEAD (SEQ ID NO: 20), IRPSKG (SEQ ID NO: 4), GGEKRD (SEQ ID NO: 5), GKKEAG (SEQ ID NO: 3), DHKL/KSG (SEQ ID NO: 21), and WEHR/KGT (SEQ ID NO: 10).

In other embodiments, the engineered proteases provided herein are specific for Factor B at a site targeted by Factor D, wherein the site targeted by Factor D comprises the amino acid sequence QQKR/KIV (SEQ ID NO: 9).

In some embodiments, the engineered proteases provided herein are based on a chymotrypsin-like serine protease of the S1A family including, but not limited to, membrane type serine protease 1 (MTSP-1), urokinase-type plasminogen activator (uPA), KLK5, and chymase. The engineered proteases of the disclosure comprise modified protease domains based on a scaffold of a serine protease, such as MTSP-1, uPA, KLK5, or chymase.

uPA-Based Engineered Proteases

In some embodiments, the engineered proteases are based on uPA, e.g. based on a uPA serine protease domain. In some embodiments, such engineered proteases are specific for Factor B at a site that is not targeted by Factor D, for example, wherein the cleavage site comprises a sequence selected from: WEHR/KGT (SEQ ID NO: 10) and KNQKR/QKQ (SEQ ID NO: 11).

In some embodiments, the uPA-based engineered proteases comprise one or more modifications with respect to a uPA comprising the amino acid sequence as set forth in SEQ ID NO: 8.

In some embodiments, the uPA-based engineered proteases comprise one or more modifications with respect to a uPA protease domain comprising the amino acid sequence as set forth in SEQ ID NO: 22.

The modifications to uPA or the uPA protease domain can be referred to by numbering the residues of the uPA protease domain by chymotrypsin numbering. Presented in Table 2 are the corresponding chymotrypsin numbers of the uPA protease domain of SEQ ID NO: 22 (equivalent to the amino acid positions 159-411 of uPA, as set forth in SEQ ID NO: 8).

Table 2 provides four rows for each amino acid. The first row lists the conventional amino acid sequence numbering of SEQ ID NO: 22, the uPA protease domain. The second row lists the conventional amino acid sequence numbering of residues 159-411 of SEQ ID NO: 8, the uPA protease domain. The third row provides the amino acid single letter abbreviation. The fourth row provides the corresponding chymotrypsin numbering below each amino acid single letter abbreviation. A residue that exists in a protease domain that does not exist in chymotrypsin is represented by a letter at the end of the notation. For example, residues in chymotrypsin that are part of a loop with amino acid 60 based on chymotrypsin numbering which are inserted into an engineered uPA are referred to as D60a, Y60b, P60c.

Table 2 provides the chymotrypsin numbering schema and its corresponding conventional numbering schema for the uPA protease domain. In subsequent tables, and throughout the disclosure, the modifications to the uPA protease domain are referred to either with chymotrypsin numbering, or with conventional amino acid numbering. If a particular modification is provided only with a chymotrypsin numbering notation, the skilled artisan will understand how to refer to Table 2 and perform the necessary conversion to understand the modification in conventional amino acid terms, and vice versa.

TABLE 2
Chymotrypsin Numbering of uPA the Protease Domain

| | | | | | | | | | | | | | | | |
|---|---|---|---|---|---|---|---|---|---|---|---|---|---|---|---|
| Conv No. | 1 | 2 | 3 | 4 | 5 | 6 | 7 | 8 | 9 | 10 | 11 | 12 | 13 | 14 | 15 |
| (SEQ ID 22) | | | | | | | | | | | | | | | |
| Conv No. | 159 | 160 | 161 | 162 | 163 | 164 | 165 | 166 | 167 | 168 | 169 | 170 | 171 | 172 | 173 |
| (SEQ ID 8) | | | | | | | | | | | | | | | |
| Amino Acid | I | I | G | G | E | F | T | T | I | E | N | Q | P | W | F |
| Chymo. No. | 16 | 17 | 18 | 19 | 20 | 21 | 22 | 23 | 24 | 25 | 26 | 27 | 28 | 29 | 30 |
| Conv No. | 16 | 17 | 18 | 19 | 20 | 21 | 22 | 23 | 24 | 25 | 26 | 27 | 28 | 29 | 30 |
| (SEQ ID 22) | | | | | | | | | | | | | | | |
| Conv No. | 174 | 175 | 176 | 177 | 178 | 179 | 180 | 181 | 182 | 183 | 184 | 185 | 186 | 187 | 188 |
| (SEQ ID 8) | | | | | | | | | | | | | | | |
| Amino Acid | A | A | I | Y | R | R | H | R | G | G | S | V | T | Y | V |
| Chymo. No. | 31 | 32 | 33 | 34 | 35 | 36 | 37 | 37A | 37b | 37c | 37d | 38 | 39 | 40 | 41 |
| Conv No. | 31 | 32 | 33 | 34 | 35 | 36 | 37 | 38 | 39 | 40 | 41 | 42 | 43 | 44 | 45 |
| (SEQ ID 22) | | | | | | | | | | | | | | | |
| Conv No. | 189 | 190 | 191 | 192 | 193 | 194 | 195 | 196 | 197 | 198 | 199 | 200 | 201 | 202 | 203 |
| (SEQ ID 8) | | | | | | | | | | | | | | | |
| Amino Acid | C | G | G | S | L | I | S | P | C | W | V | I | S | A | T |
| Chymo. No. | 42 | 43 | 44 | 45 | 46 | 47 | 48 | 49 | 50 | 51 | 52 | 53 | 54 | 55 | 56 |
| Conv No. | 46 | 47 | 48 | 49 | 50 | 51 | 52 | 53 | 54 | 55 | 56 | 57 | 58 | 59 | 60 |
| (SEQ ID 22) | | | | | | | | | | | | | | | |
| Conv No. | 204 | 205 | 206 | 207 | 208 | 209 | 210 | 211 | 212 | 213 | 214 | 215 | 216 | 217 | 218 |
| (SEQ ID 8) | | | | | | | | | | | | | | | |
| Amino Acid | H | C | F | I | D | Y | P | K | K | E | D | Y | I | V | Y |
| Chymo. No. | 57 | 58 | 59 | 60 | 60a | 60b | 60c | 61 | 62 | 62a | 63 | 64 | 65 | 66 | 67 |
| Conv No. | 61 | 62 | 63 | 64 | 65 | 66 | 67 | 68 | 69 | 70 | 71 | 72 | 73 | 74 | 75 |
| (SEQ ID 22) | | | | | | | | | | | | | | | |
| Conv No. | 219 | 220 | 221 | 222 | 223 | 224 | 225 | 226 | 227 | 228 | 229 | 230 | 231 | 232 | 233 |
| (SEQ ID 8) | | | | | | | | | | | | | | | |
| Amino Acid | L | G | R | S | R | L | N | S | N | T | Q | G | E | M | K |
| Chymo. No. | 68 | 69 | 70 | 71 | 72 | 73 | 74 | 75 | 76 | 77 | 78 | 79 | 80 | 81 | 82 |
| Conv No. | 76 | 77 | 78 | 79 | 80 | 81 | 82 | 83 | 84 | 85 | 86 | 87 | 88 | 89 | 90 |
| (SEQ ID 22) | | | | | | | | | | | | | | | |
| Conv No. | 234 | 235 | 236 | 237 | 238 | 239 | 240 | 241 | 242 | 243 | 244 | 245 | 246 | 247 | 248 |
| (SEQ ID 8) | | | | | | | | | | | | | | | |
| Amino Acid | F | E | V | E | N | L | I | L | H | K | D | Y | S | A | D |
| Chymo. No. | 83 | 84 | 85 | 86 | 87 | 88 | 89 | 90 | 91 | 92 | 93 | 94 | 95 | 96 | 97 |
| Conv No. | 91 | 92 | 93 | 94 | 95 | 96 | 97 | 98 | 99 | 100 | 101 | 102 | 103 | 104 | 105 |
| (SEQ ID 22) | | | | | | | | | | | | | | | |
| Conv No. | 249 | 250 | 251 | 252 | 253 | 254 | 255 | 256 | 257 | 258 | 259 | 260 | 261 | 262 | 263 |
| (SEQ ID 8) | | | | | | | | | | | | | | | |
| Amino Acid | T | L | A | H | H | N | D | I | A | L | L | K | I | R | S |
| Chymo. No. | 97a | 97b | 98 | 99 | 100 | 101 | 102 | 103 | 104 | 105 | 106 | 107 | 108 | 109 | 110 |
| Conv No. | 106 | 107 | 108 | 109 | 110 | 111 | 112 | 113 | 114 | 115 | 116 | 117 | 118 | 119 | 120 |
| (SEQ ID 22) | | | | | | | | | | | | | | | |
| Conv No. | 264 | 265 | 266 | 267 | 268 | 269 | 270 | 271 | 272 | 273 | 274 | 275 | 276 | 277 | 278 |
| (SEQ ID 8) | | | | | | | | | | | | | | | |
| Amino Acid | K | E | G | R | C | A | Q | P | S | R | T | I | Q | T | I |
| Chymo. No. | 110a | 110b | 110c | 110d | 111 | 112 | 113 | 114 | 115 | 116 | 117 | 118 | 119 | 120 | 121 |
| Conv No. | 121 | 122 | 123 | 124 | 125 | 126 | 127 | 128 | 129 | 130 | 131 | 132 | 133 | 134 | 135 |
| (SEQ ID 22) | | | | | | | | | | | | | | | |
| Conv No. | 279 | 280 | 281 | 282 | 283 | 284 | 285 | 286 | 287 | 288 | 289 | 290 | 291 | 292 | 293 |
| (SEQ ID 8) | | | | | | | | | | | | | | | |
| Amino Acid | C | L | P | S | M | Y | N | D | P | Q | F | G | T | S | C |
| Chymo. No. | 122 | 123 | 124 | 125 | 126 | 127 | 128 | 129 | 130 | 131 | 132 | 133 | 134 | 135 | 136 |
| Conv No. | 136 | 137 | 138 | 139 | 140 | 141 | 142 | 143 | 144 | 145 | 146 | 147 | 148 | 149 | 150 |
| (SEQ ID 22) | | | | | | | | | | | | | | | |
| Conv No. | 294 | 295 | 296 | 297 | 298 | 299 | 300 | 301 | 302 | 303 | 304 | 305 | 306 | 307 | 308 |
| (SEQ ID 8) | | | | | | | | | | | | | | | |
| Amino Acid | E | I | T | G | F | G | K | E | N | S | T | D | Y | L | Y |
| Chymo. No. | 137 | 138 | 139 | 140 | 141 | 142 | 143 | 144 | 145 | 146 | 147 | 148 | 149 | 150 | 151 |
| Conv No. | 151 | 152 | 153 | 154 | 155 | 156 | 157 | 158 | 159 | 160 | 161 | 162 | 163 | 164 | 165 |
| (SEQ ID 22) | | | | | | | | | | | | | | | |
| Conv No. | 309 | 310 | 311 | 312 | 313 | 314 | 315 | 316 | 317 | 318 | 319 | 320 | 321 | 322 | 323 |
| (SEQ ID 8) | | | | | | | | | | | | | | | |
| Amino Acid | P | E | Q | L | K | M | T | V | V | K | L | I | S | H | R |
| Chymo. No. | 152 | 153 | 154 | 155 | 156 | 157 | 158 | 159 | 160 | 161 | 162 | 163 | 164 | 165 | 166 |
| Conv No. | 166 | 167 | 168 | 169 | 170 | 171 | 172 | 173 | 174 | 175 | 176 | 177 | 178 | 179 | 180 |
| (SEQ ID 22) | | | | | | | | | | | | | | | |
| Conv No. | 324 | 325 | 326 | 327 | 328 | 329 | 330 | 331 | 332 | 333 | 334 | 335 | 336 | 337 | 338 |
| (SEQ ID 8) | | | | | | | | | | | | | | | |
| Amino Acid | E | C | Q | Q | P | H | Y | Y | G | S | E | V | T | T | K |
| Chymo. No. | 167 | 168 | 169 | 170 | 170a | 170b | 171 | 172 | 173 | 174 | 175 | 176 | 177 | 178 | 179 |
| Conv No. | 181 | 182 | 183 | 184 | 185 | 186 | 187 | 188 | 189 | 190 | 191 | 192 | 193 | 194 | 195 |
| (SEQ ID 22) | | | | | | | | | | | | | | | |
| Conv No. | 339 | 340 | 341 | 342 | 343 | 344 | 345 | 346 | 347 | 348 | 349 | 350 | 351 | 352 | 353 |
| (SEQ ID 8) | | | | | | | | | | | | | | | |
| Amino Acid | M | L | C | A | A | D | P | Q | W | K | T | D | S | C | Q |
| Chymo. No. | 180 | 181 | 182 | 183 | 184 | 185 | 185a | 185b | 186 | 187 | 188 | 189 | 190 | 191 | 192 |
| Conv No. | 196 | 197 | 198 | 199 | 200 | 201 | 202 | 203 | 204 | 205 | 206 | 207 | 208 | 209 | 210 |
| (SEQ ID 22) | | | | | | | | | | | | | | | |
| Conv No. | 354 | 355 | 356 | 357 | 358 | 359 | 360 | 361 | 362 | 363 | 364 | 365 | 366 | 367 | 368 |
| (SEQ ID 8) | | | | | | | | | | | | | | | |
| Amino Acid | G | D | S | G | G | P | L | V | C | S | L | Q | G | R | M |
| Chymo. No. | 193 | 194 | 195 | 196 | 197 | 198 | 199 | 200 | 201 | 202 | 203 | 204 | 205 | 206 | 207 |
| Conv No. | 211 | 212 | 213 | 214 | 215 | 216 | 217 | 218 | 219 | 220 | 221 | 222 | 223 | 224 | 225 |
| (SEQ ID 22) | | | | | | | | | | | | | | | |
| Conv No. | 369 | 370 | 371 | 372 | 373 | 374 | 375 | 376 | 377 | 378 | 379 | 380 | 381 | 382 | 383 |
| (SEQ ID 8) | | | | | | | | | | | | | | | |
| Amino Acid | T | L | T | G | I | V | S | W | G | R | G | C | A | L | K |
| Chymo. No. | 208 | 209 | 210 | 211 | 212 | 213 | 214 | 215 | 216 | 217 | 218 | 220 | 221 | 222 | 223 |
| Conv No. | 226 | 227 | 228 | 229 | 230 | 231 | 232 | 233 | 234 | 235 | 236 | 237 | 238 | 239 | 240 |
| (SEQ ID 22) | | | | | | | | | | | | | | | |
| Conv No. | 384 | 385 | 386 | 387 | 388 | 389 | 390 | 391 | 392 | 393 | 394 | 395 | 396 | 397 | 398 |
| (SEQ ID 8) | | | | | | | | | | | | | | | |
| Amino Acid | D | K | P | G | V | Y | T | R | V | S | H | F | L | P | W |
| Chymo. No. | 223a | 224 | 225 | 226 | 227 | 228 | 229 | 230 | 231 | 232 | 233 | 234 | 235 | 236 | 237 |
| Conv No. | 241 | 242 | 243 | 24 | 245 | 246 | 247 | 248 | 249 | 250 | 251 | 252 | 253 | | |
| (SEQ ID 22) | | | | | | | | | | | | | | | |
| Conv No. | 399 | 400 | 401 | 402 | 403 | 404 | 405 | 406 | 407 | 408 | 409 | 410 | 411 | | |
| (SEQ ID 8) | | | | | | | | | | | | | | | |
| Amino Acid | I | R | S | H | T | K | E | E | N | G | L | A | L | | |
| Chymo. No. | 238 | 239 | 240 | 241 | 242 | 243 | 244 | 245 | 246 | 247 | 248 | 249 | 250 | | |

The uPA-based engineered proteases of the disclosure comprise at least one modification of the serine protease domain of uPA. As noted above, the modifications can be any one or more of: a deletion of one or more amino acid residues, a deletion of one or more domains, a substitution of one or more amino acid residues, an insertion of one or more amino acid residues, an insertion of one or more domains, and a substitution of one or more domains. Table 3A provides exemplary modifications to the serine protease domain of uPA. By way of example, Table 3A provides three columns—the first column provides the modification using chymotrypsin numbering; the second column provides conventional amino acid sequence numbering, with respect to SEQ ID NO: 8; and the third column provides amino acid sequence numbering, with respect to SEQ ID NO: 22.

An engineered protease can be generated by the use of any one or more of the exemplary modifications provided in Table 3A. Accordingly, the uPA-based engineered protease of the disclosure may comprise any one or more of the modifications provided in Table 3A.

In some embodiments, the modification is at any one or more positions corresponding to positions G18, R36, S37, V38, Y40, D60, A96, L97, A98, H99, C122, Y151, V159, A184, Q192, R217, 1(224, using chymotrypsin numbering. By way of example, a modification to G18E is a substitution of E at the position corresponding to position 18 of the uPA serine protease domain, using chymotrypsin numbering. By way of example the modification D97delinsEG denotes a deletion of a D at residue 97, and an insertion of EG in its places, using chymotrypsin numbering. By way of example the modification L97b H99del denotes the deletion of the residues from L97b to H99, using chymotrypsin numbering.

TABLE 3A
Exemplary Modifications to the Serine Protease Domain of uPA

| | Conventional No. | Conventional No. |
|---|---|---|
| Chymotrypsin No. | (SEQ ID NO. 8) | (SEQ ID NO. 22) |
| G18E | G161E | G3E |
| R36H | R179H | R21H |
| R36S | R179S | R21S |
| S37dP | S184P | S26P |
| V38D | V185D | V27D |
| Y40H | Y187H | Y29H |
| Y40N | Y187N | Y29N |
| D60aR | D208R | D50R |
| D60aY | D208Y | D50Y |
| D60aA | D208A | D50A |
| D60aK | D208K | D50K |
| D60aH | D208H | D50H |
| L73R | L224R | L66R |
| A96del | A247del | A89del |
| A96_H99del | A247_H252del | A89_H94del |
| A96_H99delinsPGVE | A247_H252delinsPGVE | A89_H94delinsPGVE |
| A96D | A247D | A89D |
| A96E | A247E | A89E |
| A96N | A247N | A89N |
| A96G | A247G | A89G |
| L97bM | L250M | L92M |
| L97bH | L250H | L92H |
| L97bY | L250Y | L92Y |
| L97bI | L250I | L92I |
| T97a_L97bdel | T249_L250del | T91_L92del |
| T97a_H99del | T249_H252del | T91_H94del |
| D97A | D248A | D90A |
| D97E | D248E | D90E |
| D97G | D248G | D90G |
| D97L | D248L | D90L |
| D97R | D248R | D90R |
| D97T | D248T | D90T |
| D97W | D248W | D90W |
| L97bV | L250V | L92V |
| L97b_H99del | L250_H252del | L92_H94del |
| L97bV | L250V | L92V |
| L97bG | L250G | L92G |
| D97delinsEG | D249delinsEG | D90delinsEG |
| T97aS | T249S | T91S |
| A98_H99del | A251_H252del | A93_H94del |
| A98G | A251G | A93G |
| A98N | A251N | A93N |
| A98T | A251T | A93T |
| H99A | H252A | H94A |
| H99E | H252E | H94E |
| H99K | H252K | H94K |
| H99L | H252L | H94L |
| H99M | H252M | H94M |
| H99N | H252N | H94N |
| H99P | H252P | H94P |
| H99Q | H252Q | H94Q |
| H99R | H252R | H94R |
| H99S | H252S | H94S |
| H99T | H252T | H94T |
| H99V | H252V | H94V |
| H99W | H252W | H94W |
| H99Y | H252Y | H94Y |
| C122S | C279S | C121S |
| Y151N | Y308 | Y150N |
| V159A | V316A | V158A |
| Y172L | Y331L | Y173L |
| A184T | A343T | A185T |
| Q192A | Q353A | Q195A |
| Q192C | Q353C | Q195C |
| Q192D | Q353D | Q195D |
| Q192E | Q353E | Q195E |
| Q192F | Q353F | Q195F |
| Q192G | Q353G | Q195G |
| Q192H | Q353H | Q195H |
| Q192I | Q353I | Q195I |
| Q192K | Q353K | Q195K |
| Q192L | Q353L | Q195L |
| Q192M | Q353M | Q195M |
| Q192N | Q353N | Q195N |
| Q192P | Q353P | Q195P |
| Q192R | Q353R | Q195R |
| Q192S | Q353S | Q195S |
| Q192T | Q353T | Q195T |
| Q192W | Q353W | Q195W |
| Q192V | Q353V | Q195V |
| Q192Y | Q353Y | Q195Y |
| R217C | R378C | R220C |
| R217D | R378D | R220D |
| R217H | R378H | R220H |
| R217L | R378L | R220L |
| R217V | R378V | R220V |
| R217Y | R378Y | R220Y |
| K224N | K385N | K227N |
| K224V | K385V | K227V |
| K224Y | K385Y | K227Y |
| K224T | K385T | K227T |
| K224R | K385R | K227R |
| K224A | K385A | K227A |
| A98_H99delinsLK | A251_H252delinsLK | A93_H94delinsLK |
| A98_H99delinsRS | A251_H252delinsRS | A93_H94delinsRS |
| H99delinsSPG | H252delinsSPG | H94delinsSPG |

Provided in Table 3B are exemplary modifications (referred to herein as mutation strings) of the disclosure. Accordingly, provided here are uPA-based engineered proteases comprising one or more modifications provided in Table 3A. Such exemplary engineered proteases may be capable of cleaving Factor B, or display other cleavage activity. The second column provides the exemplary modification combinations using conventional numbering, with respect to SEQ ID NO: 22.

TABLE 3B
Exemplary uPA-Based Engineered Proteases

| Chymotrypsin No. | Conventional No. (SEQ ID NO. 22) |
|---|---|
| C122S | C121S |
| H99L/C122S | H94L/C121S |
| H99P/C122S | H94P/C121S |
| G18E/R36S/V38D/C122S/V159A | G3E/R21S/V27D/C121S/V158A |
| H99D/C122S | H94D/C121S |
| H99N/C122S | H94N/C121S |
| H99C/C122S | H94C/C121S |
| C122S/R217V | C121S/R220V |
| R36H/S37dP/V38D/C122S/A184T | R21H/S26P/V27D/C121S/A185T |
| V38D/A96E/D97G/C122S/Y172L/A98G/H99M/ | V27D/A89E/D90G/C121S/Y173L/A93G/H94M/ |
| T97a_L97bdel | T91_L92del |
| V38D/C122S/T97a_L97bdel | V27D/C121S/T91_L92del |
| V38D/A96G/D97E/A98G/H99V/C122S/T97a_L97bdel | V27D/A89G/D90E/A93G/H94V/C121S/T91_L92del |
| C122S/V38D/A96N/D97A/A98G/H99V/T97a_L97bdel | C121S/V27D/A89N/D90A/A93G/H94V/T91_L92del |
| V38D/C122S/D97L/A98G/H99V/T97a_L97bdel | V27D/C121S/D90L/A93G/H94V/T91_L92del |
| V38D/C122S/A96E/D97R/A98G/H99V/T97a_L97bdel | V27D/C121S/A89E/D90R/A93G/H94V/T91_L92del |
| V38D/C122S/A96E/D97H/A98G/H99L/T97a_L97bdel | V27D/C121S/A89E/D90H/A93G/H94L/T91_L92del |
| V38D/A96E/D97E/C122S/A98G/H99A/T97a_L97bdel | V27D/A89E/D90E/C121S/A93G/H94A/T91_L92del |
| V38D/C122S/A96D/D97G/A98G/H99A/T97a_L97bdel | V27D/C121S/A89D/D90G/A93F/H94A/T91_L92del |
| V38D/D97A/C122S/A98G/H99L/T97a_L97bdel | V27D/D90A/C121S/A93G/H94L/T91_L92del |
| V38D/C122S/A96D/D97W/A98N/H99L/T97a_L97bdel | V27D/C121S/A89D/D90W/A93N/H94L/T91_L92del |
| V38D/C122S/A96D/D97T/A98G/H99M/T97a_L97bdel | V27D/C121S/A89G/H94M/T91_L92del |
| V38D/D97E/L97bV/A98G/H99V/C122S/A96_H99del | V27D/D90E/L92V/A93G/H94V/C121S/A89_H94del |
| V38D/A96G/D97A/H99E/C122S/L97b_H99del | V27D/A89G/D90A/H94E/C121S/L92_H94del |
| V38D/C122S/A96_H99delinsPGVE | V27D/C121S/A89_H94delinsPGVE |
| V38D/C122S/L97b_H99del/D97delinsEG | V27D/C121S/L92 H94del/D90delinsEG |
| V38D/T97aS/L97bV/A98T/H99E/C122S/A96_H99del | V27D/T91S/L92V/A93T/H94E/C121S/A89_H94del |
| V38D/C122S/A96del/A98_H99del/-nulldelinsLK | V27D/C121S/A89del/A93_H94del/-nulldelinsLK |
| V38D/A96G/D97E/A98G/H99E/C122S/T97a_H99del | V27D/A89G/D90E/A93G/H94E/C121S/T91_H94del |
| V38D/C122S/A96del/A98_H99del/-nulldelinsRS | V27D/C121S/A89del/A93_H94del/-nulldelinsRS |
| V38D/D97E/C122S/-null_H99delinsSPG | V27D/D90E/C121S/-null_H94delinsSPG |
| Y40H/C122S | Y29H/C121S |
| V38D/A98G/H99A/C122S/T97a_L97bdel | V27D/A93G/H94A/C121S/T91_L92del |
| V38D/A98G/H99A/C122S/L97bdel | V27D/A93G/H94A/C121S/L92del |
| V38D/A98G/H99L/C122S/T97a_L97bdel | V27D/A93G/H94L/C121S/T91_L92del |

In some embodiments, a uPA-based engineered protease of the disclosure comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 8.

In some embodiments, a uPA-based engineered protease of the disclosure comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 22.

In some embodiments, a uPA-based engineered protease of the disclosure comprises a protease domain comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 22.

### MTSP-1-Based Engineered Proteases

In some embodiments, the engineered proteases are based on MTSP-1, e.g. based on a modified MTSP-1 serine protease domain. In some embodiments, such engineered proteases are specific for Factor B at a site that is not targeted by Factor D, wherein the cleavage site comprises a sequence selected from: WEHR/KGT (SEQ ID NO: 10) and KNQKR/QKQ (SEQ ID NO: 11).

In some embodiments, the engineered MTSP-1 proteases comprise one or more modifications with respect to a MTSP-1 comprising the amino acid sequence as set forth in SEQ ID NO: 18.

In some embodiments, the MTSP-1-based engineered proteases comprise one or more modifications with respect to a MTSP-1 protease domain comprising the amino acid sequence as set forth in SEQ ID NO: 7.

The modifications to the MTSP-1 or MTSP-1 protease domain can be referred to by numbering the residues of the MTSP-1 protease domain by chymotrypsin numbering. Presented in Table 4 are the corresponding chymotrypsin numbers of the MTSP-1 protease domain of SEQ ID NO: 7 (equivalent to the amino acid positions 615-855 of MTSP-1 as set forth in SEQ ID NO: 18).

Table 4 provides four rows for each amino acid. The first row lists the conventional amino acid sequence numbering of SEQ ID NO: 7, the MTSP-1 protease domain. The second row lists the conventional amino acid sequence numbering of residues 615-855 of SEQ ID NO: 18, the MTSP-1 protease domain. The third row provides the amino acid single letter abbreviation. The fourth row provides the corresponding chymotrypsin numbering below each amino acid single letter abbreviation. A residue that exists in a protease domain that does not exist in chymotrypsin is represented by a letter at the end of the notation. For example, residues in chymotrypsin that are part of a loop with amino acid 60 based on chymotrypsin numbering which are inserted into an engineered MTSP-1 are referred to as D60a and R60c.

Table 4 provides the chymotrypsin numbering schema and its corresponding conventional numbering schema for the MTSP-1 protease domain. In subsequent tables, and throughout the disclosure, the modifications to the MTSP-1 protease domain are referred to either with chymotrypsin numbering, or using conventional amino acid numbering. If a particular modification is provided only with a chymotrypsin numbering notation, the skilled artisan will understand how to refer to Table 4 and perform the necessary conversion to understand the modification in conventional amino acid terms, and vice versa.

TABLE 4
Chymotrypsin Numbering of the MTSP-1 Protease Domain

| | | | | | | | | | | | | | | | | |
|---|---|---|---|---|---|---|---|---|---|---|---|---|---|---|---|---|
| Conv No. | 1 | 2 | 3 | 4 | 5 | 6 | 7 | 8 | 9 | 10 | 11 | 12 | 13 | 14 | 15 | |
| (SEQ ID 7) | | | | | | | | | | | | | | | | |
| Conv No. | 615 | 616 | 617 | 618 | 619 | 620 | 621 | 622 | 623 | 624 | 625 | 626 | 627 | 628 | 629 | |
| (SEQ ID 18) | | | | | | | | | | | | | | | | |
| Amino Acid | V | V | G | G | T | D | A | D | E | G | E | W | P | W | Q | |
| Chymo. No. | 16 | 17 | 18 | 19 | 20 | 21 | 22 | 23 | 24 | 25 | 26 | 27 | 28 | 29 | 30 | |
| Conv No. | 16 | 17 | 18 | 19 | 20 | 21 | 22 | 23 | 24 | 25 | 26 | 27 | 28 | 29 | 30 | |
| (SEQ ID 7) | | | | | | | | | | | | | | | | |
| Conv No. | 630 | 631 | 632 | 633 | 634 | 635 | 636 | 637 | 638 | 639 | 640 | 641 | 642 | 643 | 644 | |
| (SEQ ID 18) | | | | | | | | | | | | | | | | |
| Amino Acid | V | S | L | H | A | L | G | Q | G | H | I | C | G | A | S | |
| Chymo. No. | 31 | 32 | 33 | 34 | 35 | 36 | 37 | 38 | 39 | 40 | 41 | 42 | 43 | 44 | 45 | |
| Conv No. | 31 | 32 | 33 | 34 | 35 | 36 | 37 | 38 | 39 | 40 | 41 | 42 | 43 | 44 | 45 | |
| (SEQ ID 7) | | | | | | | | | | | | | | | | |
| Conv No. | 645 | 646 | 647 | 648 | 649 | 650 | 651 | 652 | 653 | 654 | 655 | 656 | 657 | 658 | 659 | |
| (SEQ ID 18) | | | | | | | | | | | | | | | | |
| Amino Acid | L | I | S | P | N | W | L | V | S | A | A | H | C | Y | I | |
| Chymo. No. | 46 | 47 | 48 | 49 | 50 | 51 | 52 | 53 | 54 | 55 | 56 | 57 | 58 | 59 | 60 | |
| Conv No. | 46 | 47 | 48 | 49 | 50 | 51 | 52 | 53 | 54 | 55 | 56 | 57 | 58 | 59 | 60 | |
| (SEQ ID 7) | | | | | | | | | | | | | | | | |
| Conv No. | 660 | 661 | 662 | 663 | 664 | 665 | 666 | 667 | 668 | 669 | 670 | 671 | 672 | 673 | 674 | |
| (SEQ ID 18) | | | | | | | | | | | | | | | | |
| Amino Acid | D | D | R | G | F | R | Y | S | D | P | T | Q | W | T | A | |
| Chymo. No. | 60a | 60b | 60c | 60d | 60e | 60f | 60g | 60h | 60i | 61 | 62 | 63 | 64 | 65 | 66 | |
| Conv No. | 61 | 62 | 63 | 64 | 65 | 66 | 67 | 68 | 69 | 70 | 71 | 72 | 73 | 74 | 75 | |
| (SEQ ID 7) | | | | | | | | | | | | | | | | |
| Conv No. | 675 | 676 | 677 | 678 | 679 | 680 | 681 | 682 | 683 | 684 | 685 | 686 | 687 | 688 | 689 | |
| (SEQ ID 18) | | | | | | | | | | | | | | | | |
| Amino Acid | F | L | G | L | H | D | Q | S | Q | R | S | A | P | G | V | |
| Chymo. No. | 67 | 68 | 69 | 70 | 71 | 72 | 73 | 74 | 74a | 75 | 76 | 77 | 78 | 79 | 80 | |
| Conv No. | 76 | 77 | 78 | 79 | 80 | 81 | 82 | 83 | 84 | 85 | 86 | 87 | 88 | 89 | 90 | |
| (SEQ ID 7) | | | | | | | | | | | | | | | | |
| Conv No. | 690 | 691 | 692 | 693 | 694 | 695 | 696 | 697 | 698 | 699 | 700 | 701 | 702 | 703 | 704 | |
| (SEQ ID 18) | | | | | | | | | | | | | | | | |
| Amino Acid | Q | E | R | R | L | K | R | I | I | S | H | P | F | F | N | |
| Chymo. No. | 81 | 82 | 83 | 84 | 85 | 86 | 87 | 88 | 89 | 90 | 91 | 92 | 93 | 94 | 95 | |
| Conv No. | 91 | 92 | | | 94 | 95 | 96 | 97 | 98 | 99 | 100 | 101 | 102 | 103 | 104 | 105 |
| (SEQ ID 7) | | | | | | | | | | | | | | | | |
| Conv No. | 705 | 706 | | | 707 | 708 | 709 | 710 | 711 | 712 | 713 | 714 | 715 | 716 | 717 | 718 |
| (SEQ ID 18) | | | | | | | | | | | | | | | | |
| Amino Acid | D | F | T | L | T | F | D | Y | D | I | A | L | L | E | L | E |
| Chymo. No. | 96 | 97 | 97a | 97b | 98 | 99 | 100 | 101 | 102 | 103 | 104 | 105 | 106 | 107 | 108 | 109 |
| Conv No. | 106 | 107 | 108 | 109 | 110 | 111 | 112 | 113 | 114 | 115 | 116 | 117 | 118 | 119 | 120 | |
| (SEQ ID 7) | | | | | | | | | | | | | | | | |
| Conv No. | 719 | 720 | 721 | 722 | 723 | 724 | 725 | 726 | 727 | 728 | 729 | 730 | 731 | 732 | 733 | |
| (SEQ ID 18) | | | | | | | | | | | | | | | | |
| Amino Acid | K | P | A | E | Y | S | S | M | V | R | P | I | C | L | P | |
| Chymo. No. | 110 | 111 | 112 | 113 | 114 | 115 | 116 | 117 | 118 | 119 | 120 | 121 | 122 | 123 | 124 | |
| Conv No. | 121 | 122 | 123 | 124 | 125 | 126 | 127 | 128 | 129 | 130 | 131 | 132 | 133 | 134 | 135 | |
| (SEQ ID 7) | | | | | | | | | | | | | | | | |
| Conv No. | 734 | 735 | 736 | 737 | 738 | 739 | 740 | 741 | 742 | 743 | 744 | 745 | 746 | 747 | 748 | |
| (SEQ ID 18) | | | | | | | | | | | | | | | | |
| Amino Acid | D | A | S | H | V | F | P | A | G | K | A | I | W | V | T | |
| Chymo. No. | 125 | 126 | 127 | 128 | 129 | 130 | 131 | 132 | 133 | 134 | 135 | 136 | 137 | 138 | 139 | |
| Conv No. | 136 | 137 | 138 | 139 | 140 | 141 | 142 | 143 | 144 | 145 | 146 | 147 | 148 | 149 | 150 | |
| (SEQ ID 7) | | | | | | | | | | | | | | | | |
| Conv No. | 749 | 750 | 751 | 752 | 753 | 754 | 755 | 756 | 757 | | 758 | 759 | 760 | 761 | 762 | |
| (SEQ ID 18) | | | | | | | | | | | | | | | | |
| Amino Acid | G | W | G | H | T | Q | Y | G | G | | T | G | A | L | I | |
| Chymo. No. | 140 | 141 | 142 | 143 | 144 | 145 | 146 | 147 | 148 | 149 | 150 | 151 | 152 | 153 | 154 | |
| Conv No. | 151 | 152 | 153 | 154 | 155 | 156 | 157 | 158 | 159 | 160 | 161 | 162 | 163 | 164 | 165 | |
| (SEQ ID 7) | | | | | | | | | | | | | | | | |
| Conv No. | 763 | 764 | 765 | 766 | 767 | 768 | 769 | 770 | 771 | 772 | 773 | 774 | 775 | 776 | 777 | |
| (SEQ ID 18) | | | | | | | | | | | | | | | | |
| Amino Acid | L | Q | K | G | E | I | R | V | I | N | Q | T | T | C | E | |
| Chymo. No. | 155 | 156 | 157 | 158 | 159 | 160 | 161 | 162 | 163 | 164 | 165 | 166 | 167 | 168 | 169 | |
| Conv No. | 166 | 167 | 168 | 169 | 170 | 171 | 172 | 173 | 174 | 175 | 176 | 177 | 178 | 179 | 180 | |
| (SEQ ID 7) | | | | | | | | | | | | | | | | |
| Conv No. | 778 | 779 | 780 | 781 | 782 | 783 | 784 | 785 | 786 | 787 | 788 | 789 | 790 | 791 | 792 | |
| (SEQ ID 18) | | | | | | | | | | | | | | | | |
| Amino Acid | N | L | L | P | Q | Q | I | T | P | R | M | M | C | V | G | |
| Chymo. No. | 170 | 171 | 172 | 173 | 174 | 175 | 176 | 177 | 178 | 179 | 180 | 181 | 182 | 183 | 184 | |
| Conv No. | 181 | 182 | 183 | 184 | 185 | 186 | 187 | 188 | 189 | 190 | 191 | 192 | 193 | 194 | 195 | |
| (SEQ ID 7) | | | | | | | | | | | | | | | | |
| Conv No. | 793 | 794 | 795 | 796 | 797 | 798 | 799 | 800 | 801 | 802 | 803 | 804 | 805 | 806 | 807 | |
| (SEQ ID 18) | | | | | | | | | | | | | | | | |
| Amino Acid | F | L | S | G | G | V | D | S | C | Q | G | D | S | G | G | |
| Chymo. No. | 184a | 185 | 186 | 186a | 187 | 188 | 189 | 190 | 191 | 192 | 193 | 194 | 195 | 196 | 197 | |
| Conv No. | 196 | 197 | 198 | 199 | 200 | 201 | 202 | 203 | 204 | 205 | 206 | 207 | 208 | 209 | 210 | |
| (SEQ ID 7) | | | | | | | | | | | | | | | | |
| Conv No. | 808 | 809 | 810 | 811 | 812 | 813 | 814 | 815 | 816 | 817 | 818 | 819 | 820 | 821 | 822 | |
| (SEQ ID 18) | | | | | | | | | | | | | | | | |
| Amino Acid | P | L | S | S | V | E | A | D | G | R | I | F | Q | A | G | |
| Chymo. No. | 198 | 199 | 200 | 201 | 202 | 203 | 204 | 204a | 205 | 206 | 207 | 208 | 209 | 210 | 211 | |
| Conv No. | 211 | 212 | 213 | 214 | 215 | 216 | 217 | 218 | 219 | 220 | 221 | 222 | 223 | 224 | 225 | |
| (SEQ ID 7) | | | | | | | | | | | | | | | | |
| Conv No. | 823 | 824 | 825 | 826 | 827 | 828 | 829 | 830 | 831 | 832 | 833 | 834 | 835 | 836 | 837 | |
| (SEQ ID 18) | | | | | | | | | | | | | | | | |
| Amino Acid | V | V | S | W | G | D | G | C | A | Q | R | N | K | P | G | |
| Chymo. No. | 212 | 213 | 214 | 215 | 216 | 217 | 218 | 219 | 220 | 221 | 222 | 223 | 224 | 225 | 226 | |
| Conv No. | 226 | 227 | 228 | 229 | 230 | 231 | 232 | 233 | 234 | 235 | 236 | 237 | 238 | 239 | 240 | |
| (SEQ ID 7) | | | | | | | | | | | | | | | | |
| Conv No. | 838 | 839 | 840 | 841 | 842 | 843 | 844 | 845 | 846 | 847 | 848 | 849 | 850 | 851 | 852 | |
| (SEQ ID 18) | | | | | | | | | | | | | | | | |
| Amino Acid | V | Y | T | R | L | P | L | F | R | D | W | I | K | E | N | |
| Chymo. No. | 227 | 228 | 229 | 230 | 231 | 232 | 233 | 234 | 235 | 236 | 237 | 238 | 239 | 240 | 241 | |
| Conv No. | 241 | 242 | 243 | | | | | | | | | | | | | |
| (SEQ ID 7) | | | | | | | | | | | | | | | | |
| Conv No. | 853 | 854 | 855 | | | | | | | | | | | | | |
| (SEQ ID 18) | | | | | | | | | | | | | | | | |
| Amino Acid | T | G | V | | | | | | | | | | | | | |
| Chymo. No. | 242 | 243 | 244 | | | | | | | | | | | | | |

The MTSP-1-based engineered proteases of the disclosure comprise at least one modification of the serine protease domain of MTSP-1. As noted above, the modifications can be any one or more of: a deletion of one or more amino acid residues, a deletion of one or more domains, a substitution of one or more amino acid residues, an insertion of one or more amino acid residues, an insertion of one or more domains, and a substitution of one or more domains. Table 5A provides exemplary modifications to the serine protease domain of MTSP-1. By way of example, Table 5A provides three columns—the first column provides the modification using chymotrypsin numbering; the second column provides conventional amino acid sequence numbering, with respect to SEQ ID NO: 18; and the third column provides amino acid sequence numbering, with respect to SEQ ID NO: 7.

The modifications to MTSP-1 or the MTSP-1 protease domain can be referred to by numbering the residues of the MTSP-1 protease domain by chymotrypsin numbering. Presented in Table 4 are the corresponding chymotrypsin numbers of the MTSP-1 protease domain of SEQ ID NO: 7 (equivalent to the amino acid positions 615-855 of MTSP-1, as set forth in SEQ ID NO: 18).

An engineered protease can be generated by the use of any one or more of the exemplary modifications provided in Table 5A. Accordingly, the MTSP-1-based engineered protease of the disclosure may comprise any one or more of the modifications provided in Table 5A.

In some embodiments, the modification is at any one or more positions corresponding to positions D23, 141, L70, A77, F94, D96, F97, T98, F99, K110, C122, D125, Y146, Q175, V183, Q192, A204, D217, and K224, using chymotrypsin numbering. By way of example, a modification to F99S of MTSP-1 is a substitution modification at the position corresponding to position 99 of the MTSP-1 serine protease domain, using chymotrypsin numbering.

TABLE 5A
Exemplary Modifications to the Serine
Protease Domain of MTSP-1

| | Conventional No. | Conventional No. |
|---|---|---|
| Chymotrypsin No. | (SEQ ID NO. 18) | (SEQ ID NO. 7) |
| D23Y | D622Y | D8Y |
| I41A | I640A | I26A |
| I41F | I640F | I26F |
| I41G | I640G | I26G |
| I41K | I640K | I26K |
| I41N | I640N | I26N |
| I41Q | I640Q | I26Q |
| I41R | I640R | I26R |
| I41S | I640S | I26S |
| L70Q | L678Q | L64 |
| A77T | A686T | A72T |
| F94Y | F703Y | F89Y |
| D96A | D705A | D91A |
| D96P | D705P | D9P1 |
| D96S | D705S | D91S |
| D96T | D705T | D91T |
| D96insD | D705insD | D91insD |
| F97A | F706A | F92A |
| F97D | F706D | F92D |
| F97E | F706E | F92E |
| F97G | F706G | F92G |
| F97K | F706K | F92K |
| F97L | F706L | F92L |
| F97M | F706M | F92M |
| F97R | F706R | F92R |
| F97S | F706S | F92S |
| F97T | F706T | F92T |
| ins 97aA | ins 706aA | ins 93A |
| ins 97aS | ins 706aS | ins 93S |
| ins 97aC | ins 706aC | ins 93C |
| ins 97bG | ins 706bG | ins 93aG |
| ins 97bL | ins 706bL | ins 93aL |
| ins 97bN | ins 706bN | ins 93aN |
| ins 97aV | ins 706aV | ins 93V |
| ins 97aY | ins 706aY | ins 93Y |
| ins 97bS | ins 706bS | ins 93aS |
| ins 97bT | ins 706bT | ins 93aT |
| T98insA | T707insA | T94insA |
| T98insAA | T707insAA | T94insAA |
| T98A | T707A | T94A |
| T98G | T707G | T94G |
| T98P | T707P | T94P |
| T98S | T707S | T94S |
| T98V | T707V | T94V |
| F99H | F708H | F95H |
| F99I | F708I | F95I |
| F99L | F708L | F95L |
| F99M | F708M | F95M |
| F99N | F708N | F95N |
| F99P | F708P | F95P |
| F99S | F708S | F95S |
| F99V | F708V | F95V |
| F99Y | F708Y | F95Y |
| K110I | K719I | K106I |
| C122S | C731S | C118S |
| D125G | D734G | D121G |
| Y146D | Y755D | Y142D |
| Y146E | Y755E | Y142E |
| R161G | R769G | R157G |
| L172M | L780M | L168M |
| Q175H | Q783H | Q171H |
| Q175L | Q783L | Q171L |
| Q175N | Q783N | Q171N |
| Q175P | Q783P | Q171P |
| Q175R | Q783R | Q171R |
| Q175S | Q783S | Q171S |
| Q175T | Q783T | Q171T |
| Q175W | Q783W | Q171W |
| V183A | V791A | V179A |
| Q192M | Q802M | Q190M |
| Q192R | Q802R | Q190R |
| Q192T | Q802T | Q190T |
| Q192V | Q802V | Q190V |
| G197A | G807A | G195A |
| A204V | A814V | A202V |
| D217F | D828F | D216F |
| D217I | D828I | D216I |
| D217M | D828M | D216M |
| D217T | D828T | D216T |
| D217Y | D828Y | D216Y |
| D217V | D828V | D216V |
| D217Y | D828Y | D216Y |
| K224A | K835A | K223A |
| K224G | K835G | K223G |
| K224L | K835L | K223L |
| K224M | K835M | K223M |
| K224N | K835N | K223N |
| K224Q | K835Q | K223Q |
| K224R | K835R | K223R |
| K224T | K835T | K223T |
| K224Y | K835Y | K223Y |

Provided in Table 5B are exemplary modifications (mutation strings)of the disclosure. Accordingly, provided here are MTSP-1-based engineered proteases, comprising one or more mutation strings provided in Table 5B. Such exemplary engineered proteases may be capable of cleaving Factor B, or display other cleavage activity. Residues that are noted in brackets, such as C[17] and C[19], refer to residues that are part of the chain of the protease in zymogen form, which is later cleaved and does not remain in the mature protease.

TABLE 5B
Exemplary MTSP-1-Based Engineered Proteases
Chymotrypsin No.

C122S
Y146E/K224N
F97E/K224N/F99L/D217I/C122S/C[17]S/C[19]S
C[17]S/C[19]S/F97E/F99L/C122S/Q175L/Q192M/D217I/K224R
C[17]S/C[19]S/F97M/F99L/C122S/Q175W/D217I/K224A
F97E/K224N/F99L/D217I/C122S
F97E/F99L/C122S/Q175L/Q192M/D217I/K224R
F97M/F99L/C122S/Q175W/D217I/K224A
I41G/F97D/F99L/C122S
A77aT/F94Y/C122S/D125G
I41A/F97D/F99L/C122S
I41S/F97D/F99V/C122S/Y146D/C[17]S/C[19]S
I41G/F97D/F99L/C122S/Y146D/C[17]S/C[19]S
I41G/F97E/F99L/C122S/C[17]S/C[19]S
I41N/F97D/F99L/C122S/C[17]S/C[19]S
I41A/F97D/F99V/C122S/Y146D/Q175T/C[17]S/C[19]S
I41G/F97D/F99M/C122S/C[17]S/C[19]S
F97L/T98G/F99V/C122S/C[17]S/C[19]S/-null_D96insD
C122S/C[17]S/C[19]S/-null_T98insA
C122S/C[17]S/C[19]S/-null_T98insAA
C[17]S/C[19]S/I41Q/F97T/F99L/C122S/Y146D/Q175W
C[17]S/C[19]S/I41F/F97D/F99I/C122S/Y146D/Q175S/D217T
C[17]S/C[19]S/I41R/F97D/F99N/C122S/Y146D/D217V/K224Q
C[17]S/C[19]S/I41R/F97E/F99N/C122S/Y146D/D217Y/K224M
C[17]S/C[19]S/I41G/F97E/F99L/C122S/R161G/D217Y/K224Q
C[17]S/C[19]S/I41R/F97D/F99H/C122S/Y146D/Q175P/Q192M/D217F/
K224L
C[17]S/C[19]S/I41R/F97E/F99S/C122S/Y146D/Q175R/D217I/K224M
C[17]S/C[19]S/I41K/F97D/F99Y/C122S
I41K/F97D/F99H/C122S/C[17]S/C[19]S
F94Y/F99L/C122S/G197A/C[17]S/C[19]S
F99L/C122S/L172M/G197A/C[17]S/C[19]S
D23Y/L70Q/F99L/K110I/C122S/V183A/A204V/C[17]S/C[19]S
F94Y/C122S/C[17]S/C[19]S
I41A/F97D/F99L/C122S
I41N/F97D/F99L/C122S/C[17]S/C[19]S
I41S/F97D/F99V/C122S/Y146D
I41G/F97D/F99L/C122S/Y146D/
I41G/F97E/F99L/C122S
I41N/F97D/F99L/C122S
I41A/F97D/F99V/C122S/Y146D/Q175T
I41G/F97D/F99M/C122S
F97L/T98G/F99V/C122S/-null_D96insD
C122S/-null_T98insA
C122S/-null_T98insAA
I41Q/F97T/F99L/C122S/Y146D/Q175W
I41F/F97D/F99I/C122S/Y146D/Q175S/D217T
I41R/F97D/F99N/C122S/Y146D/D217V/K224Q
I41R/F97E/F99N/C122S/Y146D/D217Y/K224M
I41G/F97E/F99L/C122S/R161G/D217Y/K224Q
I41R/F97D/F99H/C122S/Y146D/Q175P/Q192M/D217F/K224L
I41R/F97E/F99S/C122S/Y146D/Q175R/D217I/K224M
I41K/F97D/F99Y/C122S
I41K/F97D/F99H/C122S
F94Y/F99L/C122S/G197A
F99L/C122S/L172M/G197A
D23Y/L70Q/F99L/K110I/C122S/V183A/A204V
F94Y/C122S
I41A/F97D/F99L/C122S
I41N/F97D/F99L/C122S
Y146E/K224N

In some embodiments, a MTSP-1-based engineered protease of the disclosure comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 18.

In some embodiments, a MTSP-1-based engineered protease of the disclosure comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 7.

In some embodiments, a MTSP-1-based engineered protease of the disclosure comprises a protease domain comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 7.

### Chymase-Based Engineered Proteases

In some embodiments, the engineered proteases are based on chymase, e.g. based on a modified chymase serine protease domain. In some embodiments, such engineered proteases are specific for Factor B at a site that is not targeted by Factor D, wherein the cleavage site comprises a sequence selected from: DVFY/QMI (SEQ ID NO: 12), EGVD/AE (SEQ ID NO: 13), DHKL/KSG (SEQ ID NO: 14), and TPW/SLA (SEQ ID NO: 15).

In some embodiments, the chymase-based engineered proteases comprise one or more modifications with respect to a chymase protease domain comprising the amino acid sequence as set forth in SEQ ID NO: 6.

The modifications to the chymase or the chymase protease domain can be referred to by numbering the residues of the chymase protease domain by chymotrypsin numbering. Presented in Table 6 are the corresponding chymotrypsin numbers of amino acid positions 1-226 of the chymase protease domain of SEQ ID NO: 6.

Table 6 provides three rows for each amino acid. The first row lists the conventional amino acid sequence numbering of SEQ ID NO: 6, the chymase protease domain. The second row provides the amino acid single letter abbreviation. The third row provides the corresponding chymotrypsin numbering of the chymase protease domain below each amino acid single letter abbreviation. A residue that exists in a protease domain that does not exist in chymotrypsin is represented by a letter at the end of the notation. For example, residues in chymotrypsin at amino acid 36 based on chymotrypsin numbering which are inserted into an engineered chymase are referred to as V36a, S36b, and N36c.

Table 6 provides the chymotrypsin numbering schema and its corresponding conventional numbering schema for the Chymase protease domain. In subsequent tables, and throughout the disclosure, the modifications to the Chymase protease domain are referred to either with chymotrypsin numbering, or using conventional amino acid numbering. If a particular modification is provided only with a chymotrypsin numbering notation, the skilled artisan will understand how to refer to Table 6 and perform the necessary conversion to understand the modification in conventional amino acid terms, and vice versa.

TABLE 6
Chymotrypsin Numbering of the Chymase Protease Domain

| | | | | | | | | | | | | | | | |
|---|---|---|---|---|---|---|---|---|---|---|---|---|---|---|---|
| Conv No. | 1 | 2 | 3 | 4 | 5 | 6 | 7 | 8 | 9 | 10 | 11 | 12 | 13 | 14 | 15 |
| (SEQ ID 6) | | | | | | | | | | | | | | | |
| Amino Acid | I | I | G | G | T | E | C | K | P | H | S | R | P | Y | M |
| Chymo. No. | 16 | 17 | 18 | 19 | 20 | 21 | 22 | 23 | 24 | 25 | 26 | 27 | 28 | 29 | 30 |
| Conv No. | 16 | 17 | 18 | 19 | 20 | 21 | 22 | 23 | 24 | 25 | 26 | 27 | 28 | 29 | 30 |
| (SEQ ID 6) | | | | | | | | | | | | | | | |
| Amino Acid | A | Y | L | E | I | V | T | S | N | G | P | S | K | F | C |
| Chymo. No. | 31 | 32 | 33 | 34 | 35 | 36 | 36a | 36b | 36c | 37 | 38 | 39 | 40 | 41 | 42 |
| Conv No. | 31 | 32 | 33 | 34 | 35 | 36 | 37 | 38 | 39 | 40 | 41 | 42 | 43 | 44 | 45 |
| (SEQ ID 6) | | | | | | | | | | | | | | | |
| Amino Acid | G | G | F | L | I | R | R | N | F | V | L | T | A | A | H |
| Chymo. No. | 43 | 44 | 45 | 46 | 47 | 48 | 49 | 50 | 51 | 52 | 53 | 54 | 55 | 56 | 57 |
| Conv No. | 46 | 47 | 48 | 49 | 50 | 51 | 52 | 53 | 54 | 55 | 56 | 57 | 58 | 59 | 60 |
| (SEQ ID 6) | | | | | | | | | | | | | | | |
| Amino Acid | C | A | G | R | S | I | T | V | T | L | G | A | H | N | I |
| Chymo. No. | 58 | 59 | 50 | 61 | 63 | 64 | 65 | 66 | 67 | 68 | 69 | 70 | 71 | 72 | 73 |
| Conv No. | 61 | 62 | 63 | 64 | 65 | 66 | 67 | 68 | 69 | 70 | 71 | 72 | 73 | 74 | 75 |
| (SEQ ID 6) | | | | | | | | | | | | | | | |
| Amino Acid | T | E | E | E | D | T | W | Q | K | L | E | V | I | K | Q |
| Chymo. No. | 74 | 75 | 77 | 78 | 79 | 79a | 80 | 81 | 82 | 83 | 84 | 85 | 86 | 87 | 88 |
| Conv No. | 76 | 77 | 78 | 79 | 80 | 81 | 82 | 83 | 84 | 85 | 86 | 87 | 88 | 89 | 90 |
| (SEQ ID 6) | | | | | | | | | | | | | | | |
| Amino Acid | F | R | H | P | K | Y | N | T | S | T | L | H | H | D | I |
| Chymo. No. | 89 | 90 | 91 | 92 | 93 | 94 | 95 | 96 | 97 | 98 | 99 | 100 | 101 | 102 | 103 |
| Conv No. | 91 | 92 | 93 | 94 | 95 | 96 | 97 | 98 | 99 | 100 | 101 | 102 | 103 | 104 | 105 |
| (SEQ ID 6) | | | | | | | | | | | | | | | |
| Amino Acid | M | L | L | K | L | K | E | K | A | S | L | T | L | A | V |
| Chymo. No. | 104 | 105 | 106 | 107 | 108 | 109 | 110 | 111 | 112 | 113 | 114 | 115 | 116 | 117 | 118 |
| Conv No. | 106 | 107 | 108 | 109 | 110 | 111 | 112 | 113 | 114 | 115 | 116 | 117 | 118 | 119 | 120 |
| (SEQ ID 6) | | | | | | | | | | | | | | | |
| Amino Acid | G | T | L | P | F | P | S | Q | F | N | F | V | P | P | G |
| Chymo. No. | 119 | 120 | 121 | 122 | 123 | 124 | 125 | 126 | 127 | 128 | 129 | 130 | 131 | 132 | 133 |
| Conv No. | 121 | 122 | 123 | 124 | 125 | 126 | 127 | 128 | 129 | 130 | 131 | 132 | 133 | 134 | 135 |
| (SEQ ID 6) | | | | | | | | | | | | | | | |
| Amino Acid | R | M | C | R | V | A | G | W | G | R | T | G | V | L | K |
| Chymo. No. | 134 | 135 | 136 | 137 | 138 | 139 | 140 | 141 | 142 | 143 | 144 | 145 | 146 | 147 | 148 |
| Conv No. | 136 | 137 | 138 | 139 | 140 | 141 | 142 | 143 | 144 | 145 | 146 | 147 | 148 | 149 | 150 |
| (SEQ ID 6) | | | | | | | | | | | | | | | |
| Amino Acid | P | G | S | D | T | L | Q | E | V | K | L | R | L | M | D |
| Chymo. No. | 150 | 151 | 152 | 153 | 154 | 155 | 156 | 157 | 158 | 159 | 160 | 161 | 162 | 163 | 164 |
| Conv No. | 151 | 152 | 153 | 154 | 155 | 156 | 157 | 158 | 159 | 160 | 161 | 162 | 163 | 164 | 165 |
| (SEQ ID 6) | | | | | | | | | | | | | | | |
| Amino Acid | P | Q | A | C | S | H | F | R | D | F | D | H | N | L | Q |
| Chymo. No. | 165 | 166 | 167 | 168 | 169 | 172 | 173 | 174 | 175 | 176 | 177 | 177a | 178 | 179 | 180 |
| Conv No. | 166 | 167 | 168 | 169 | 170 | 171 | 172 | 173 | 174 | 175 | 176 | 177 | 178 | 179 | 180 |
| (SEQ ID 6) | | | | | | | | | | | | | | | |
| Amino Acid | L | C | V | G | N | P | R | K | T | K | S | A | F | K | G |
| Chymo. No. | 181 | 182 | 183 | 184 | 185 | 185a | 185b | 186 | 187 | 188 | 189 | 190 | 191 | 192 | 193 |
| Conv No. | 181 | 182 | 183 | 184 | 185 | 186 | 187 | 188 | 189 | 190 | 191 | 192 | 193 | 194 | 195 |
| (SEQ ID 6) | | | | | | | | | | | | | | | |
| Amino Acid | D | S | G | G | P | L | L | C | A | G | V | A | Q | G | I |
| Chymo. No. | 194 | 195 | 196 | 197 | 198 | 199 | 200 | 201 | 202 | 207 | 208 | 209 | 210 | 211 | 212 |
| Conv No. | 196 | 197 | 198 | 199 | 200 | 201 | 202 | 203 | 204 | 205 | 206 | 207 | 208 | 209 | 210 |
| (SEQ ID 6) | | | | | | | | | | | | | | | |
| Amino Acid | V | S | Y | G | R | S | D | A | K | P | P | A | V | F | T |
| Chymo. No. | 213 | 214 | 215 | 216 | 217 | 218 | 219 | 220 | 221 | 224 | 225 | 226 | 227 | 228 | 229 |
| Conv No. | 211 | 212 | 213 | 214 | 215 | 216 | 217 | 218 | 219 | 220 | 221 | 222 | 223 | 224 | 225 |
| (SEQ ID 6) | | | | | | | | | | | | | | | |
| Amino Acid | R | I | S | H | Y | R | P | W | I | N | Q | I | L | Q | A |
| Chymo. No. | 230 | 231 | 232 | 233 | 234 | 235 | 236 | 237 | 238 | 239 | 240 | 241 | 242 | 243 | 244 |
| Conv No. | 226 | | | | | | | | | | | | | | |
| (SEQ ID 6) | | | | | | | | | | | | | | | |
| Amino Acid | N | | | | | | | | | | | | | | |
| Chymo. No. | 245 | | | | | | | | | | | | | | |

The chymase-based engineered proteases of the disclosure comprise at least one modification of the serine protease domain of chymase. As noted above, the modifications can be any one or more of: a deletion of one or more amino acid residues, a deletion of one or more domains, a substitution of one or more amino acid residues, an insertion of one or more amino acid residues, an insertion of one or more domains, and a substitution of one or more domains. Table 7A provides exemplary modifications to the serine protease domain of chymase. By way of example, Table 7A provides two columns—the first column provides the modifications using chymotrypsin numbering; the second column provides conventional amino acid sequence numbering, with respect to SEQ ID NO: 6.

An engineered protease can be generated by the use of any one or more of the exemplary modifications provided in Table 7A. Accordingly, the chymase-based engineered protease of the disclosure may comprise any one or more of the modifications provided in Table 7A.

In some embodiments, the modification is at any one or more positions corresponding to positions C22, S36, P38, G43, R49, K87, K93, 1103, L114, L116, F123, V138, F173, D175, S189, A190, F191, K192, L199, V213, G216, A220, A226, F228, using chymotrypsin numbering. By way of example, a modification to C22S of chymase is a substitution modification at the position corresponding to position 22 of the chymase serine protease domain, using chymotrypsin numbering.

TABLE 7A
Exemplary Modifications to the Serine
Protease Domain of Chymase

| Chymotrypsin No. | Conventional No. (SEQ ID NO. 6) |
|---|---|
| C22S | C7S |
| G43S | G31S |
| R49P | R37P |
| S36bN | S23N |
| P38A | P26A |
| P38Q | P26Q |
| P38S | P26S |
| K87E | K74E |
| K93E | K80E |
| K93M | K80M |
| L99G | L86G |
| I103V | 190V |
| L114P | L101P |
| F123L | F110L |
| V138A | V125A |
| V138I | V125I |
| V138L | V125L |
| V138M | V125M |
| V138S | V125S |
| L116P | L103P |
| G145D | G132D |
| L160V | L146V |
| F173A | F157A |
| F173G | F157G |
| F173H | F157H |
| F173L | F157L |
| F173R | F157R |
| F173Y | F157Y |
| S189A | S176A |
| S189E | S176E |
| S189G | S176G |
| S189K | S176K |
| S189L | S176L |
| S189M | S176M |
| S189Q | S176Q |
| S189R | S176R |
| S189T | S176T |
| S189V | S176V |
| S189Y | S176Y |
| A190D | A177D |
| A190E | A177E |
| A190H | A177H |
| A190I | A177I |
| A190L | A177L |
| A190M | A177M |
| A190P | A177P |
| A190S | A177S |
| A190T | A177T |
| A190V | A177V |
| F191A | F178A |
| F191D | F178D |
| F191E | F178E |
| F191G | F178G |
| F191H | F178H |
| F191I | F178I |
| F191L | F178L |
| F191Q | F178Q |
| F191R | F178R |
| F191S | F178S |
| F191T | F178T |
| F191V | F178V |
| F191W | F178W |
| F191Y | F178Y |
| K192A | K179A |
| K192I | K179I |
| K192L | K179L |
| K192M | K179M |
| K192R | K179R |
| K192S | K179S |
| L199V | L186V |
| V213A | V196A |
| V213G | V196G |
| Y215F | Y198F |
| Y215S | Y198S |
| Y215L | Y198L |
| Y215T | Y198T |
| G216A | G199A |
| G216I | G199I |
| G216L | G199L |
| G216M | G199M |
| G216N | G199N |
| G216Q | G199Q |
| G216S | G199S |
| G216T | G199T |
| G216V | G199V |
| G216Y | G199Y |
| R217A | R200A |
| R217G | R200G |
| R217K | R200K |
| R217P | R200P |
| S218D | S201D |
| S218F | S201F |
| S218H | S201H |
| S218M | S201M |
| A220D | A203D |
| A220E | A203E |
| A220G | A203G |
| A220H | A203H |
| A220I | A203I |
| A220K | A203K |
| A220L | A203L |
| A220M | A203M |
| A220N | A203N |
| A220Q | A203Q |
| A220R | A203R |
| A220T | A203T |
| A220V | A203V |
| A220W | A203W |
| A220Y | A203Y |
| A226D | A207D |
| A226E | A207E |
| A226F | A207F |
| A226G | A207G |
| A226I | A207I |
| A226L | A207L |
| A226M | A207M |
| A226N | A207N |
| A226R | A207R |
| A226S | A207S |
| A226T | A207T |
| A226V | A207V |
| A226Y | A207Y |
| F228A | F209A |
| L99H | L86S |
| Y215H | Y198H |
| R217T | R200T |
| S218V | S201V |
| F173I | F157I |
| Y215K | Y198K |
| S218I | S201I |
| F41I | F29I |
| L99W | L86W |
| G151L | G137L |
| F173K | F157K |
| R217S | R200S |
| F41R | F29R |
| G151T | G137T |
| Y215R | Y198R |
| R217M | R200M |
| F41V | F29V |
| L99Y | L86Y |
| L99F | L86F |
| G151R | G137R |
| G151V | G137V |
| L99R | L86R |
| S218L | S201L |
| K40L | K28L |
| F41A | F29A |
| F41G | F29G |
| K40M | K28L |
| S218R | S201R |
| L99Q | L86Q |
| G151S | G137S |
| G151K | G137K |
| F173S | F157S |
| F41K | F29K |
| G151A | G137A |
| F41L | F29L |
| K93Q | K80Q |
| G119D | G106D |
| H100P | H87P |
| L116R | L103R |
| Q180H | Q165H |
| R235S | R216S |
| E21G | E6G |
| R61H | R49H |
| M135T | M122T |
| D175N | D159N |
| A209V | A192V |
| K111E | K98E |
| P150S | P136S |
| S152G | S138G |
| Q166H | Q152H |
| D175N | D159N |
| A220S | A203S |
| L99N | L86N |
| G151P | G137P |
| F41H | F29H |
| S218E | S201E |
| V183I | V168I |
| F228V | F209V |
| V138T | V125T |
| A190G | A177G |
| F228M | F209M |
| D175S | D159S |
| D175W | D159W |
| D175R | D159R |
| S218T | S201T |
| D175Y | D159Y |
| V213Q | V196Q |
| K40A | K28A |
| L99M | L86M |
| D175H | D159H |
| D175T | D159T |
| F173M | F157M |
| D175T | D159T |
| D175A | D159A |
| K40V | K28V |
| F41T | F29T |
| D175K | D159K |
| D175Q | D159Q |
| F41Q | F29Q |
| D175W | D159W |
| F41H | F29H |
| D175S | D159S |
| S218N | S201N |
| K40Q | K28Q |
| K40I | K28I |
| D175F | D159F |
| L99S | L86S |
| G151H | G137H |

Provided in Table 7B are exemplary modifications (mutation strings) of the disclosure. Accordingly, provided here are chymase-based engineered proteases, comprising one or more modifications (mutation strings) provided in Table 7A. Such exemplary engineered proteases may be capable of cleaving Factor B, or display other cleavage activity. As examples, engineered chymase-based engineered proteases include proteases having the exemplary combination of modifications: C22S/P38Q/K40M/F41R/V138I/F173Y/D175R/A190SN213A/S218V/A226R, and C22S/P38Q/K40M/F41H/V138I/F173Y/D175R/A190SN213A/S218V/A226R, based on chymotrypsin numbering.

TABLE 7B
Exemplary Chymase-Based Engineered Proteases

| Chymotrypsin No. | Conventional No. (SEQ ID NO. 6) |
|---|---|
| C22S | C7S |
| C22S/A226R | C7S/A207R |
| C22S/L99H/F173L/K192R/Y215H/R217T/S218V/ | C7S/L86H/F157L/K179R/Y198H/R200T/S201V/A207R |
| A226R | |
| C22S/L99H/F173I/K192R/Y215K/R217T/S218I/ | C7S/L86H/F157I/K179R/Y198K/R200T/S201I/A207R |
| A226R | |
| C22S/L99H/F173L/K192R/Y215H/R217T/S218V/ | C7S/L86H/F157L/K179R/Y198H/R200T/S201V/A207R |
| A226R | |
| C22S/F41I/L99W/G151L/F173K/R217S/A226R | C7S/F29I/L86W/G137L/F157K/R200S/A207R |
| C22S/F41R/L99G/G151T/F173Y/Y215R/R217M/ | C7S/F29R/L86G/G137T/F157Y/Y198R/R200M/A207R |
| A226R | |
| C22S/F41R/L99H/G151T/F173Y/Y215K/R217M/ | C7S/F29R/L86H/G137T/F157Y/Y198K/R200M/A207R |
| A226R | |
| C22S/F41R/L99Y/G151T/F173K/R217S/A226R | C7S/F29R/L86Y/G137T/F157K/R200S/A207R |
| C22S/F41V/L99F/G151R/F173K/R217S/A226R | C7S/F29V/L86F/G137R/F157K/R200S/A207R |
| C22S/F41V/L99H/G151R/F173L/Y215R/R217T/ | C7S/F29V/L86H/G137R/F157L/Y198R/R200T/A207R |
| A226R | |
| F41V/L99H/G151V/F173Y/Y215K/R217G/A226R | F29V/L86H/G137V/F157Y/Y198K/R200G/A207R |
| C22S/F41V/L99W/G151L/F173K/R217S/A226R | C7S/F29V/L86W/G137L/F157K/R200S/A207R |
| C22S/K192R/A226R | C7S/K179R/A207R |
| C22S/L99R/F173L/K192R/Y215H/R217T/S218V/ | C7S/L86R/F157L/K179R/Y198H/R200T/S201V/A207R |
| A226R | |
| C22S/L99Y/F173L/K192R/Y215H/R217T/S218V/ | C7S/L86Y/F157L/K179R/Y198H/R200T/S201V/A207R |
| A226R | |
| C22S/L99H/F173Y/K192R/S218L/A226R | C7S/L86H/F157Y/K179R/S201L/A207R |
| C22S/L99R/F173Y/K192R/S218L/A226R | C7S/L86R/F157Y/K179R/S201L/A207R |
| C22S/L99Y/F173Y/K192R/S218L/A226R | C7S/L86Y/F157Y/K179R/S201L/A207R |
| C22S/K40L/F41A/K192R/A226R | C7S/K28L/F29A/K179R/A207R |
| C22S/K40L/F41G/K192R/A226R | C7S/K28L/F29G/K179R/A207R |
| C22S/K40M/F41V/K192R/A226R | C7S/K28L/F29V/K179R/A207R |
| C22S/K40M/F41V/G151R/K192R/A226R | C7S/K28L/F29V/G137R/K179R/A207R |
| C22S/K40L/F41V/G151R/K192R/A226R | C7S/K28L/F29V/G137R/K179R/A207R |
| C22S/L99H/F173Y/K192R/Y215K/R217S/S218V/ | C7S/L86H/F157Y/K179R/Y198K/R200S/S201V/A207R |
| A226R | |
| C22S/L99F/F173K/K192R/R217S/S218I/A226R | C7S/L86F/F157K/K179R/R200S/S201I/A207R |
| C22S/L99W/F173K/K192R/R217S/S218V/A226R | C7S/L86W/F157K/K179R/R200S/S201V/A207R |
| C22S/L99W/F173K/R217S/S218R/A226R | C7S/L86W/F157K/R200S/S201R/A207R |
| C22S/L99Q/F173L/Y215K/R217T/S218L/A226R | C7S/L86Q/F157L/Y198K/R200T/S201L/A207R |
| C22S/F41R/L99H/G151S/F173Y/Y215K/R217M/ | C7S/F29R/L86H/G137S/F157Y/Y198K/R200M/A207R |
| A226R | |
| C22S/F41I/L99H/G151K/F173Y/Y215K/R217G/ | C7S/F29I/L86H/G137K/F157Y/Y198K/R200G/A207R |
| A226R | |
| C22S/F41V/L99H/G151R/F173S/Y215K/A226R | C7S/F29V/L86H/G137R/F157S/Y198K/A207R |
| C22S/F41R/L99H/F173Y/Y215K/R217M/A226R | C7S/F29R/L86H/F157Y/Y198K/R200M/A207R |
| C22S/F41K/L99H/G151A/F173Y/Y215K/R217S/ | C7S/F29K/L86H/G137A/F157Y/Y198K/R200S/A207R |
| A226R | |
| C22S/F41R/L99H/G151A/F173S/Y215K/R217T/ | C7S/F29R/L86H/G137A/F157S/Y198K/R200T/A207R |
| A226R | |
| C22S/F41L/L99H/G151T/F173Y/Y215K/A226R | C7S/F29L/L86H/G137T/F157Y/Y198K/A207R |
| C22S/F41R/L99H/G151T/F173Y/Y215K/A226R | C7S/F29R/L86H/G137T/F157Y/Y198K/A207R |
| C22S/F41G/L99H/G151T/F173Y/Y215K/A226R | C7S/F29G/L86H/G137T/F157Y/Y198K/A207R |
| C22S/F41R/L99H/F173Y/Y215K/A226R | C7S/F29R/L86H/F157Y/Y198K/A207R |
| C22S/F41V/L99H/F173Y/Y215K/A226R | C7S/F29V/L86H/F157Y/Y198K/A207R |
| C22S/F41G/L99H/F173Y/Y215K/A226R | C7S/F29G/L86H/F157Y/Y198K/A207R |
| C22S/F41R/L99H/G151T/F173L/Y215K/A226R | C7S/F29R/L86H/G137T/F157L/Y198K/A207R |
| C22S/F41V/L99H/G151T/F173L/Y215K/A226R | C7S/F29V/L86H/G137T/F157L/Y198K/A207R |
| C22S/F41G/L99H/G151T/F173L/Y215K/A226R | C7S/F29G/L86H/G137T/F157L/Y198K/A207R |
| C22S/F41R/L99H/F173L/Y215K/A226R | C7S/F29R/L86H/F157L/Y198K/A207R |
| C22S/F41V/L99H/G151T/F173Y/Y215K/A226R | C7S/F29V/L86H/G137T/F157Y/Y198K/A207R |
| C22S/F41L/L99H/F173Y/Y215K/A226R | C7S/F29L/L86H/F157Y/Y198K/A207R |
| C22S/F41L/L99H/G151T/F173L/Y215K/A226R | C7S/F29L/L86H/G137T/F157L/Y198K/A207R |
| C22S/F41L/L99H/F173L/Y215K/A226R | C7S/F29L/L86H/F157L/Y198K/A207R |
| C22S/F41V/L99H/F173L/Y215K/A226R | C7S/F29V/L86H/F157L/Y198K/A207R |
| C22S/F41G/L99H/F173L/Y215K/A226R | C7S/F29G/L86H/F157L/Y198K/A207R |
| C22S/L99R/K192R/S218L/A226R | C7S/L86R/K179R/S201L/A207R |
| C22S/L99R/F173Y/S218L/A226R | C7S/L86R/F157Y/S201L/A207R |
| C22S/L99R/F173Y/K192R/A226R | C7S/L86R/F157Y/K179R/A207R |
| C22S/K93Q/G119D/F173S/K192R/A226R | C7S/K80Q/G106D/F157S/K179R/A207R |
| C22S/P38Q/H100P/L116R/F123L/Q180H/S218I/ | C7S/P26Q/H87P/L103R/F110L/Q165H/S2011/A207R/ |
| A226R/R235S | R216S |
| E21G/C22S/R61H/M135T/F173Y/D175N/A209V/ | E6G/C7S/R49H/M122T/F157Y/D159N/A192V/A207R |
| A226R | |
| C22S/K111E/F173L/Y215H/A226R | C7S/K98E/F157L/Y198H/A207R |
| C22S/F173L/Y215H/A226R | C7S/F157L/Y198H/A207R |
| C22S/M135T/P150S/S152G/Q166H/D175N/A190T/ | C7S/M122T/P136S/S138G/Q152H/D159N/A177T/ |
| A226R | A207R |
| C22S/P38Q/H100P/L116R/F123L/Q180H/A226R/ | C7S/P26Q/H87P/L103R/F110L/Q165H/A207R/R216S |
| R235S | |
| C22S/V138I/L160V/S189T/A226R | C7S/V125I/L146V/S176T/A207R |
| C22S/V138L/A226R | C7S/V125L/A207R |
| C22S/V138L/A226R | C7S/V125L/A207R |
| C22S/L160V/S189T/A226R | C7S/L146V/S176T/A207R |
| C22S/S189T/A226R | C7S/S176T/A207R |
| C22S/S218I/A226R | C7S/S201I/A207R |
| C22S/F173Y/A226R | C7S/F157Y/A207R |
| C22S/D175N/A226R | C7S/D159N/A207R |
| C22S/F173Y/D175N/A226R | C7S/F157Y/D159N/A207R |
| C22S/F173Y/D175N/A226R | C7S/F157Y/D159N/A207R |
| C22S/A220S/A226R | C7S/A203S/A207R |
| C22S/S189T/A190S/F191S/A220L/A226R | C7S/S176T/A177S/A178S/A203L/A207R |
| C22S/K40L/F41R/L99N/F173Y/S218L/A226R | C7S/K28L/F29R/L86N/F157Y/S201L/A207R |
| C22S/K40M/F41R/L99H/G151P/F173S/S218V/ | C7S/K28L/F29R/L86H/G137P/F157S/S201V/A207R |
| A226R | |
| C22S/K40M/F41R/L99H/G151A/F173S/S218V/ | C7S/K28L/F29R/L86H/G137A/F157S/S201V/A207R |
| A226R | |
| C22S/K40M/F41I/L99H/G151R/F173S/S218L/ | C7S/K28L/F29I/L86H/G137R/F157S/S201L/A207R |
| A226R | |
| C22S/K40M/F41R/L99N/G151S/F173Y/S218L/ | C7S/K28L/F29R/L86N/G137S/F157Y/S201L/A207R |
| A226R | |
| C22S/K40M/F41R/L99N/G151P/F173S/S218V/ | C7S/K28L/F29R/L86N/G137P/F157S/S201V/A207R |
| A226R | |
| C22S/K40M/F41R/L99H/G151P/F173S/S218I/ | C7S/K28L/F29R/L86H/G137P/F157S/S201I/A207R |
| A226R | |
| C22S/K40L/F41H/L99N/F173Y/S218L/A226R | C7S/K28L/F29H/L86N/F157Y/S201L/A207R |
| C22S/K40M/F41V/L99N/G151R/F173S/S218V/ | C7S/K28L/F29V/L86N/G137R/F157S/S201V/A207R |
| A226R | |
| C22S/K40M/F41I/L99N/G151R/F173S/S218V/ | C7S/K28L/F29I/L86N/G137R/F157S/S201V/A207R |
| A226R | |
| C22S/F41I/L99H/G151R/F173S/S218L/A226R | C7S/F29I/L86H/G137R/F157S/S201L/A207R |
| C22S/K40L/F41L/L99N/G151H/F173Y/S218V/ | C7S/K28L/F29L/L86N/G137H/F157Y/S201V/A207R |
| A226R | |
| C22S/K40L/F41K/L99H/G151S/F173Y/S218L/ | C7S/K28L/F29K/L86H/G137S/F157Y/S201L/A207R |
| A226R | |
| C22S/K40L/F41K/L99H/F173Y/S218L/A226R | C7S/K28L/F29K/L86H/F157Y/S201L/A207R |
| C22S/K40L/F41K/L99N/F173Y/S218L/A226R | C7S/K28L/F29K/L86N/F157Y/S201L/A207R |
| C22S/K40M/F41V/L99N/G151R/F173S/S218E/ | C7S/K28L/F29V/L86N/G137R/F157S/S201E/A207R |
| A226R | |
| C22S/A190S/A226R | C7S/A177S/A207R |
| C22S/S189T/A190S/A226R | C7S/S176T/A177S/A207R |
| C22S/K40L/F41R/L99N/F173Y/D175N/S218L/ | C7S/K28L/F29R/L86N/F157Y/D159N/S201L/A207R |
| A226R | |
| C22S/K40L/F41H/L99N/F173Y/D175N/S218L/ | C7S/K28L/F29H/L86N/F157Y/D159N/S201L/A207R |
| A226R | |
| C22S/K40L/F41K/L99N/F173Y/D175N/S218L/ | C7S/K28L/F29K/L86N/F157Y/D159N/S201L/A207R |
| A226R | |
| C22S/P38Q/V138I/V183I/L199V/A226R/F228V | C7S/P26Q/V125I/V168I/L186V/A207R/F209V |
| C22S/V138T/A190S/V213A/A226R | C7S/V125T/A177S/V196A/A207R |
| C22S/P38Q/V138L/A190G/L199V/V213A/A226R/ | C7S/P26Q/V125L/A177G/L186V/V196A/A207R/ |
| F228A | F209A |
| C22S/P38Q/V138I/A226R/F228A | C7S/P26Q/V125I/A207R/F209A |
| C22S/F41K/L99N/F173Y/D175N/S218L/A226R | C7S/F29K/L86N/F157Y/D159N/S201L/A207R |
| C22S/K40L/L99N/F173Y/D175N/S218L/A226R | C7S/K28L/L86N/F157Y/D159N/S201L/A207R |
| C22S/K40L/F41K/F173Y/D175N/S218L/A226R | C7S/K28L/F29K/F157Y/D159N/S201L/A207R |
| C22S/K40L/F41K/L99N/D175N/S218L/A226R | C7S/K28L/F29K/L86N/D159N/S201L/A207R |
| C22S/K40L/F41K/L99N/F173Y/D175N/A226R | C7S/K28L/F29K/L86N/F157Y/D159N/A207R |
| C22S/V138A/G145D/S189T/A190S/A226R | C7S/V125A/G132D/S176T/A177S/A207R |
| C22S/A226R/F228A | C7S/A207R/F209A |
| C22S/V138A/S189T/A226R/F228M | C7S/V125A/S176T/A207R/F209M |
| C22S/V138L/S189T/A190G/A226R | C7S/V125L/S176T/A177G/A207R |
| C22S/V138S/S189T/A226R/F228M | C7S/V125S/S176T/A207R/F209M |
| C22S/S189T/A226R/F228M | C7S/S176T/A207R/F209M |
| C22S/V138A/A226R | C7S/V125A/A207R |
| C22S/V138A/S189T/A226R | C7S/V125A/S176T/A207R |
| C22S/K40M/F41R/L99N/F173Y/D175S/S218L/ | C7S/K28L/F29R/L86N/F157Y/D159S/S201L/A207R |
| A226R | |
| C22S/K40M/F41R/L99H/F173Y/D175W/S218L/ | C7S/K28L/F29R/L86H/F157Y/D159W/S201L/A207R |
| A226R | |
| C22S/K40M/F41K/F173Y/D175R/S218E/A226R | C7S/K28L/F29K/F157Y/D159R/S201E/A207R |
| C22S/K40M/F41K/L99N/F173Y/D175R/S218T/ | C7S/K28L/F29K/L86N/F157Y/D159R/S201T/A207R |
| A226R | |
| C22S/K40L/F41K/L99Q/F173Y/D175N/S218V/ | C7S/K28L/F29K/L86Q/F157Y/D159N/S201V/A207R |
| A226R | |
| C22S/K40M/F41R/L99Q/F173Y/D175R/S218V/ | C7S/K28L/F29R/L86Q/F157Y/D159R/S201V/A207R |
| A226R | |
| C22S/K40L/F41K/L99H/F173Y/D175S/S218I/ | C7S/K28L/F29K/L86H/F157Y/D159S/S201I/A207R |
| A226R | |
| C22S/K40L/F41R/L99G/F173Y/D175R/S218V/ | C7S/K28L/F29R/L86G/F157Y/D159R/S201V/A207R |
| A226R | |
| C22S/K40L/F41R/L99H/F173Y/D175Y/S218I/ | C7S/K28L/F29R/L86H/F157Y/D159Y/S201I/A207R |
| A226R | |
| C22S/K40M/F41R/L99Q/F173Y/D175R/S218I/ | C7S/K28L/F29R/L86Q/F157Y/D159R/S201I/A207R |
| A226R | |
| C22S/P38Q/V138I/A190S/V213A/A226R | C7S/P26Q/V125I/A177S/V196A/A207R |
| C22S/P38Q/V213Q/A226R/F228A | C7S/P26Q/V196Q/A207R/F209A |
| C22S/P38Q/K40A/F41R/L99H/F173Y/D175Y/ | C7S/P26Q/K28A/F29R/L86H/F157Y/D159Y/V196Q/ |
| V213Q/S218V/A226R/F228A | S201V/A207R/F209A |
| C22S/P38Q/K40A/F41R/L99H/D175N/V213Q/ | C7S/P26Q/K28A/F29R/L86H/D159N/V196Q/S201I/ |
| S218I/A226R/F228A | A207R/F209A |
| C22S/P38Q/K40M/F41R/L99M/F173Y/D175N/ | C7S/P26Q/K28L/F29R/L86M/F157Y/D159N/V196Q/ |
| V213Q/S218V/A226R/F228A | S201V/A207R/F209A |
| C22S/P38Q/K40M/F41R/D175N/V213Q/S218V/ | C7S/P26Q/K28L/F29R/D159N/V196Q/S201V/A207R/ |
| A226R/F228A | F209A |
| C22S/P38Q/K40L/F41R/V138I/F173Y/D175N/ | C7S/P26Q/K28L/F29R/V125I/F157Y/D159N/A177S/ |
| A190S/V213A/S218V/A226R | V196A/S201V/A207R |
| C22S/P38Q/K40L/F41R/L99H/V138I/F173Y/ | C7S/P26Q/K28L/F29R/L86H/V125I/F157Y/D159S/ |
| D175S/A190S/V213A/S218I/A226R | A177S/V196A/S201I/A207R |
| C22S/P38Q/K40M/F41R/L99F/V138I/F173S/ | C7S/P26Q/K28L/F29R/L86F/V125I/F157S/D159H/ |
| D175H/A190S/V213A/S218V/A226R | A177S/V196A/S201V/A207R |
| C22S/P38Q/K40A/F41R/L99H/V138I/F173Y/ | C7S/P26Q/K28A/F29R/L86H/V125I/F157Y/D159N/ |
| D175N/A190S/V213A/S218V/A226R | A177S/V196A/S201V/A207R |
| C22S/P38Q/K40A/F41K/L99H/V138I/D175S/ | C7S/P26Q/K28A/F29K/L86H/V125I/D159S/A177S/ |
| A190S/V213A/S218V/A226R | V196A/S201V/A207R |
| C22S/P38Q/K40M/F41R/L99N/V138I/F173Y/ | C7S/P26Q/K28L/F29R/L86N/V125I/F157Y/D159T/ |
| D175T/A190S/V213A/S218V/A226R | A177S/V196A/S201V/A207R |
| C22S/P38Q/K40M/F41R/V138I/F173Y/D175R/ | C7S/P26Q/K28L/F29R/V125I/F157Y/D159R/A177S/ |
| A190S/V213A/S218V/A226R | V196A/S201V/A207R |
| C22S/P38Q/K40L/F41R/L99N/V138I/F173Y/ | C7S/P26Q/K28L/F29R/L86N/V125I/F157Y/D159N/ |
| D175N/A190S/V213A/S218V/A226R | A177S/V196A/S201V/A207R |
| C22S/P38Q/K40A/F41R/V138I/D175R/A190S/ | C7S/P26Q/K28A/F29R/V125I/D159R/A177S/V196A/ |
| V213A/S218T/A226R | S201T/A207R |
| C22S/P38Q/K40A/F41R/L99H/V138I/F173Y/ | C7S/P26Q/K28A/F29R/L86H/V125I/F157Y/A177S/ |
| A190S/V213A/S218V/A226R | V196A/S201V/A207R |
| C22S/P38Q/K40M/F41H/L99N/V138I/F173Y/ | C7S/P26Q/K28L/F29H/L86N/V125I/F157Y/D159S/ |
| D175S/A190S/V213A/S218T/A226R | A177S/V196A/S201T/A207R |
| C22S/P38Q/K40M/F41K/L99Y/V138I/F173M/ | C7S/P26Q/K28L/F29K/L86Y/V125I/F157M/D159Y/ |
| D175Y/A190S/V213A/S218V/A226R | A177S/V196A/S201V/A207R |
| C22S/P38Q/K40A/F41R/V138I/F173S/D175T/ | C7S/P26Q/K28A/F29R/V125I/F157S/D159T/A177S/ |
| A190S/V213A/S218V/A226R | V196A/S201V/A207R |
| C22S/P38Q/K40L/F41R/V138I/F173L/D175H/ | C7S/P26Q/K28L/F29R/V125I/F157L/D159H/A177S/ |
| A190S/V213A/S218V/A226R | V196A/S201V/A207R |
| C22S/P38Q/K40M/F41R/L99N/V138I/D175N/ | C7S/P26Q/K28L/F29R/L86N/V125I/D159N/A177S/ |
| A190S/V213A/S218T/A226R | V196A/S201T/A207R |
| C22S/P38Q/K40M/F41H/V138I/F173Y/D175R/ | C7S/P26Q/K28L/F29H/V125I/F157Y/D159R/A177S/ |
| A190S/V213A/S218V/A226R | V196A/S201V/A207R |
| C22S/P38Q/K40M/F41R/L99Q/V138I/F173Y/ | C7S/P26Q/K28L/F29R/L86Q/V125I/F157Y/D159A/ |
| D175A/A190S/V213A/S218T/A226R | A177S/V196A/S201T/A207R |
| C22S/P38Q/K40A/F41R/L99H/V138I/D175R/ | C7S/P26Q/K28A/F29R/L86H/V125I/D159R/A177S/ |
| A190S/V213A/S218L/A226R | V196A/S201L/A207R |
| C22S/P38Q/K40M/F41R/L99F/V138I/F173L/ | C7S/P26Q/K28L/F29R/L86F/V125I/F157L/D159N/ |
| D175N/A190S/V213A/A226R | A177S/V196A/A207R |
| C22S/P38Q/K40M/F41R/L99Y/V138I/F173L/ | C7S/P26Q/K28L/F29R/L86Y/V125I/F157L/D159S/ |
| D175S/A190S/V213A/S218V/A226R | A177S/V196A/S201V/A207R |
| C22S/P38Q/K40L/F41R/V138I/D175N/A190S/ | C7S/P26Q/K28L/F29R/V125I/D159N/A177S/V196A/ |
| V213A/S218V/A226R | S201V/A207R |
| C22S/P38Q/K40M/F41R/L99Y/V138I/F173Y/ | C7S/P26Q/K28L/F29R/L86Y/V125I/F157Y/D159S/ |
| D175S/A190S/V213A/S218V/A226R | A177S/V196A/S201V/A207R |
| C22S/P38Q/K40L/F41R/V138I/F173M/D175N/ | C7S/P26Q/K28L/F29R/V125I/F157M/D159N/A177S/ |
| A190S/V213A/S218L/A226R | V196A/S201L/A207R |
| C22S/P38Q/K40L/F41R/L99N/V138I/F173Y/ | C7S/P26Q/K28L/F29R/L86N/V125I/F157Y/D159T/ |
| D175T/A190S/V213A/S218V/A226R | A177S/V196A/S201V/A207R |
| C22S/P38Q/K40M/F41R/V138I/F173L/D175N/ | C7S/P26Q/K28L/F29R/V125I/F157L/D159N/A177S/ |
| A190S/V213A/S218L/A226R | V196A/S201L/A207R |
| C22S/P38Q/K40L/F41K/L99N/V138I/F173Y/ | C7S/P26Q/K28L/F29K/L86N/V125I/F157Y/D159S/ |
| D175S/A190S/V213A/S218I/A226R | A177S/V196A/S201I/A207R |
| C22S/P38Q/K40A/F41R/L99Y/V138I/F173L/ | C7S/P26Q/K28A/F29R/L86Y/V125I/F157L/D159R/ |
| D175R/A190S/V213A/S218L/A226R | A177S/V196A/S201L/A207R |
| C22S/P38Q/K40L/F41R/L99Q/V138I/F173Y/ | C7S/P26Q/K28L/F29R/L86Q/V125I/F157Y/D159N/ |
| D175N/A190S/V213A/S218V/A226R | A177S/V196A/S201V/A207R |
| C22S/P38Q/K40M/F41R/L99Q/V138I/F173Y/ | C7S/P26Q/K28L/F29R/L86Q/V125I/F157Y/D159N/ |
| D175N/A190S/V213A/S218V/A226R | A177S/V196A/S201V/A207R |
| C22S/P38Q/K40V/F41R/V138I/F173L/D175R/ | C7S/P26Q/K28V/F29R/V125I/F157L/D159R/A177S/ |
| A190S/V213A/S218I/A226R | V196A/S201I/A207R |
| C22S/P38Q/K40M/F41T/V138I/D175R/A190S/ | C7S/P26Q/K28L/F29T/V125I/D159R/A177S/V196A/ |
| V213A/S218I/A226R | S201I/A207R |
| C22S/P38Q/K40M/F41R/V138I/F173L/D175K/ | C7S/P26Q/K28L/F29R/V125I/F157L/D159K/A177S/ |
| A190S/V213A/S218V/A226R | V196A/S201V/A207R |
| C22S/P38Q/K40M/F41R/L99Y/V138I/F173S/ | C7S/P26Q/K28L/F29R/L86Y/V125I/F157S/D159S/ |
| D175S/A190S/V213A/S218T/A226R | A177S/V196A/S201T/A207R |
| C22S/P38Q/K40A/F41T/L99Y/V138I/F173S/ | C7S/P26Q/K28A/F29T/L86Y/V125I/F157S/D159H/ |
| D175H/A190S/V213A/S218I/A226R | A177S/V196A/S201I/A207R |
| C22S/P38Q/K40A/F41R/V138I/F173L/D175R/ | C7S/P26Q/K28A/F29R/V125I/F157L/D159R/A177S/ |
| A190S/V213A/S218L/A226R | V196A/S201L/A207R |
| C22S/P38Q/K40M/F41R/L99Y/V138I/F173L/ | C7S/P26Q/K28L/F29R/L86Y/V125I/F157L/D159Q/ |
| D175Q/A190S/V213A/S218T/A226R | A177S/V196A/S201T/A207R |
| C22S/P38Q/K40A/F41Q/V138I/F173S/D175W/ | C7S/P26Q/K28A/F29Q/V125I/F157S/D159W/A177S/ |
| A190S/V213A/S218V/A226R | V196A/S201V/A207R |
| C22S/P38Q/K40M/F41H/V138I/D175S/A190S/ | C7S/P26Q/K28L/F29H/V125I/D159S/A177S/V196A/ |
| V213A/A226R | A207R |
| C22S/P38Q/K40V/F41R/V138I/F173L/D175N/ | C7S/P26Q/K28V/F29R/V125I/F157L/D159N/A177S/ |
| A190S/V213A/S218V/A226R | V196A/S201V/A207R |
| C22S/P38Q/K40V/F41R/L99H/V138I/F173M/ | C7S/P26Q/K28V/F29R/L86H/V125I/F157M/D159N/ |
| D175N/A190S/V213A/S218T/A226R | A177S/V196A/S201T/A207R |
| C22S/P38Q/K40L/F41R/V138I/F173L/D175R/ | C7S/P26Q/K28L/F29R/V125I/F157L/D159R/A177S/ |
| A190S/V213A/S218V/A226R | V196A/S201V/A207R |
| C22S/P38Q/K40L/F41R/L99M/V138I/F173L/ | C7S/P26Q/K28L/F29R/L86M/V125I/F157L/D159R/ |
| D175R/A190S/V213A/S218V/A226R | A177S/V196A/S201V/A207R |
| C22S/P38Q/K40A/F41R/L99H/V138I/F173M/ | C7S/P26Q/K28A/F29R/L86H/V125I/F157M/D159T/ |
| D175T/A190S/V213A/S218I/A226R | A177S/V196A/S201I/A207R |
| C22S/P38Q/K40M/F41R/L99Y/V138I/F173L/ | C7S/P26Q/K28L/F29R/L86Y/V125I/F157L/D159S/ |
| D175S/A190S/V213A/S218T/A226R | A177S/V196A/S201T/A207R |
| C22S/P38Q/K40A/F41R/L99H/V138I/F173Y/ | C7S/P26Q/K28A/F29R/L86H/V125I/F157Y/D159N/ |
| D175N/A190S/V213A/S218I/A226R | A177S/V196A/S201I/A207R |
| C22S/P38Q/K40M/F41R/L99W/V138I/F173M/ | C7S/P26Q/K28L/F29R/L86W/V125I/F157M/D159H/ |
| D175H/A190S/V213A/S218T/A226R | A177S/V196A/S201T/A207R |
| C22S/P38Q/K40M/F41H/L99H/V138I/D175S/ | C7S/P26Q/K28L/F29H/L86H/V125I/D159S/A177S/ |
| A190S/V213A/S218V/A226R | V196A/S201V/A207R |
| C22S/P38Q/K40V/F41R/V138I/F173S/D175N/ | C7S/P26Q/K28V/F29R/V125I/F157S/D159N/A177S/ |
| A190S/V213A/S218I/A226R | V196A/S201I/A207R |
| C22S/P38Q/K40L/F41R/L99F/V138I/F173L/ | C7S/P26Q/K28L/F29R/L86F/V125I/F157L/D159N/ |
| D175N/A190S/V213A/A226R | A177S/V196A/A207R |
| C22S/P38Q/K40M/F41R/L99H/V138I/D175Q/ | C7S/P26Q/K28L/F29R/L86H/V125I/D159Q/A177S/ |
| A190S/V213A/S218V/A226R | V196A/S201V/A207R |
| C22S/P38Q/K40L/F41R/V138I/F173Y/D175H/ | C7S/P26Q/K28L/F29R/V125I/F157Y/D159H/A177S/ |
| A190S/V213A/S218T/A226R | V196A/S201T/A207R |
| C22S/P38Q/K40M/F41K/L99W/V138I/F173M/ | C7S/P26Q/K28L/F29K/L86W/V125I/F157M/A177S/ |
| A190S/V213A/S218N/A226R | V196A/S201N/A207R |
| C22S/P38Q/K40Q/F41R/V138I/F173Y/D175N/ | C7S/P26Q/K28Q/F29R/V125I/F157Y/D159N/A177S/ |
| A190S/V213A/S218V/A226R | V196A/S201V/A207R |
| C22S/P38Q/K40I/F41R/L99N/V138I/D175T/ | C7S/P26Q/K28I/F29R/L86N/V125I/D159T/A177S/ |
| A190S/V213A/S218V/A226R | V196A/S201V/A207R |
| C22S/P38Q/K40Q/F41R/L99H/V138I/F173Y/ | C7S/P26Q/K28Q/F29R/L86H/V125I/F157Y/D159S/ |
| D175S/A190S/V213A/S218V/A226R | A177S/V196A/S201V/A207R |
| C22S/P38Q/K40A/F41R/L99H/V138I/F173Y/ | C7S/P26Q/K28A/F29R/L86H/V125I/F157Y/D159N/ |
| D175N/A190S/V213A/S218L/A226R | A177S/V196A/S201L/A207R |
| C22S/P38Q/K40M/F41R/L99N/V138I/F173Y/ | C7S/P26Q/K28L/F29R/L86N/V125I/F157Y/D159F/ |
| D175F/A190S/V213A/S218V/A226R | A177S/V196A/S201V/A207R |
| C22S/P38Q/K40M/F41R/L99N/V138I/F173Y/ | C7S/P26Q/K28L/F29R/L86N/V125I/F157Y/D159W/ |
| D175W/A190S/V213A/S218T/A226R | A177S/V196A/S201T/A207R |
| C22S/P38Q/K40L/F41R/L99Y/V138I/F173Y/ | C7S/P26Q/K28L/F29R/L86Y/V125I/F157Y/D159N/ |
| D175N/A190S/V213A/S218V/A226R | A177S/V196A/S201V/A207R |
| C22S/P38Q/K40M/F41R/L99S/V138I/F173Y/ | C7S/P26Q/K28L/F29R/L86S/V125I/F157Y/D159N/ |
| D175N/A190S/V213A/S218V/A226R | A177S/V196A/S201V/A207R |
| C22S/P38Q/K40A/F41R/L99H/V138I/D175N/ | C7S/P26Q/K28A/F29R/L86H/V125I/D159N/A177S/ |
| A190S/V213A/S218I/A226R | V196A/S201I/A207R |
| C22S/P38Q/K40M/F41R/V138I/F173S/D175Y/ | C7S/P26Q/K28L/F29R/V125I/F157S/D159Y/A177S/ |
| A190S/V213A/S218V/A226R | V196A/S201V/A207R |
| C22S/P38Q/K40M/F41R/V138I/F173L/D175R/ | C7S/P26Q/K28L/F29R/V125I/F157L/D159R/A177S/ |
| A190S/V213A/S218T/A226R | V196A/S201T/A207R |
| C22S/P38Q/K40L/F41R/V138I/F173Y/D175R/ | C7S/P26Q/K28L/F29R/V125I/F157Y/D159R/A177S/ |
| A190S/V213A/S218T/A226R | V196A/S201T/A207R |
| C22S/P38Q/K40A/F41K/L99N/V138I/D175H/ | C7S/P26Q/K28A/F29K/L86N/V125I/D159H/A177S/ |
| A190S/V213A/S218V/A226R | V196A/S201V/A207R |
| C22S/P38Q/K40M/F41R/V138I/F173Y/D175Y/ | C7S/P26Q/K28L/F29R/V125I/F157Y/D159Y/A177S/ |
| A190S/V213A/S218I/A226R | V196A/S201I/A207R |

In some embodiments, a chymase-based engineered protease of the disclosure comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 6.

In some embodiments, a chymase-based engineered protease of the disclosure comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 6.

In some embodiments, a chymase-based engineered protease of the disclosure comprises a protease domain comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 6.

### KLK5-Based Engineered Proteases

In some embodiments, the engineered proteases are based on KLK5. In some embodiments, the engineered proteases are based on KLK5, and are specific for Factor B at a site that is targeted by Factor D, wherein the cleavage site comprises the amino acid sequence QQKR/KIV (SEQ ID NO: 9).

In some embodiments, the KLK5-based engineered proteases are based on a KLK5 comprising the amino acid sequence as set forth in SEQ ID NO: 23. Residues within or modifications to the KLK5 can be referred to by numbering the residues of KLK5 by chymotrypsin numbering. Presented in Table 7C is the chymotrypsin numbering scheme of amino acid positions 45-271 of KLK5 SEQ ID NO: 28, as set forth in SEQ ID NO: 23. Table 7C lists the amino acid residues 45-271 of SEQ ID NO: 23 above the amino acid single letter abbreviation, and the corresponding chymotrypsin numbering below each amino acid single letter abbreviation. A residue that exists in a protease that does not exist in a chymotrypsin is represented by a letter at the end of the notation. For example, residues in chymotrypsin at amino acid 36 based on chymotrypsin numbering which are inserted into an engineered KLK5 are referred to as 36a, 36b, 36c.

Table 7C provides the chymotrypsin numbering schema and its corresponding conventional numbering schema for the KLK5 protease domain. In subsequent tables, and throughout the disclosure, the modifications to the KLK5 protease domain are referred to either with chymotrypsin numbering, or using conventional amino acid numbering. If a particular modification is provided only with a chymotrypsin numbering notation, the skilled artisan will understand how to refer to Table 7C and perform the necessary conversion to understand the modification in conventional amino acid terms, and vice versa.

TABLE 7C
Chymotrypsin Numbering of KLK5

| | | | | | | | | | | | | | | | |
|---|---|---|---|---|---|---|---|---|---|---|---|---|---|---|---|
| Conv No. | 45 | 46 | 47 | 48 | 49 | 50 | 51 | 52 | 53 | 54 | 55 | 56 | 57 | 58 | 59 |
| (SEQ ID 23) | | | | | | | | | | | | | | | |
| Amino Acid | I | I | N | G | S | D | C | D | M | H | T | Q | P | W | Q |
| Chymo. No. | 16 | 17 | 18 | 19 | 20 | 21 | 22 | 23 | 24 | 25 | 26 | 27 | 28 | 29 | 30 |
| Conv No. | 60 | 61 | 62 | 63 | 64 | 65 | 66 | 67 | 68 | 69 | 70 | 71 | 72 | 73 | 74 |
| (SEQ ID 23) | | | | | | | | | | | | | | | |
| Amino Acid | A | A | L | L | L | R | P | N | Q | L | Y | C | G | A | V |
| Chymo. No. | 31 | 32 | 33 | 34 | 35 | 36 | 37 | 38 | 39 | 40 | 41 | 42 | 43 | 44 | 45 |
| Conv No. | 75 | 76 | 77 | 78 | 79 | 80 | 81 | 82 | 83 | 84 | 85 | 86 | 87 | 88 | 89 |
| (SEQ ID 23) | | | | | | | | | | | | | | | |
| Amino Acid | L | V | H | P | Q | W | L | L | T | A | A | H | C | R | K |
| Chymo. No. | 46 | 47 | 48 | 49 | 50 | 51 | 52 | 53 | 54 | 55 | 56 | 57 | 58 | 59 | 60 |
| Conv No. | 90 | 91 | 92 | 93 | 94 | 95 | 96 | 97 | 98 | 99 | 100 | 101 | 102 | 103 | 104 |
| (SEQ ID 23) | | | | | | | | | | | | | | | |
| Amino Acid | K | V | F | R | V | R | L | G | H | Y | S | L | S | P | V |
| Chymo. No. | 61 | 62 | 63 | 64 | 65 | 66 | 67 | 69 | 70 | 71 | 72 | 73 | 74 | 74A | 75 |
| Conv No. | 105 | 106 | 107 | 108 | 109 | 110 | 111 | 112 | 113 | 114 | 115 | 116 | 117 | 118 | 119 |
| (SEQ ID 23) | | | | | | | | | | | | | | | |
| Amino Acid | Y | E | S | G | Q | Q | M | F | Q | G | V | K | S | I | P |
| Chymo. No. | 76 | 77 | 78 | 79 | 80 | 81 | 82 | 83 | 84 | 85 | 86 | 87 | 88 | 89 | 90 |
| Conv No. | 120 | 121 | 122 | 123 | 124 | 125 | 126 | 127 | 128 | 129 | 130 | 131 | 132 | 133 | 134 |
| (SEQ ID 23) | | | | | | | | | | | | | | | |
| Amino Acid | H | P | G | Y | S | H | P | G | H | S | N | D | L | M | L |
| Chymo. No. | 91 | 92 | 93 | 94 | 95 | 96 | 97 | 98 | 99 | 100 | 101 | 102 | 103 | 104 | 105 |
| Conv No. | 135 | 136 | 137 | 138 | 139 | 140 | 141 | 142 | 143 | 144 | 145 | 146 | 147 | 148 | 149 |
| (SEQ ID 23) | | | | | | | | | | | | | | | |
| Amino Acid | I | K | L | N | R | R | I | R | P | T | K | D | V | R | P |
| Chymo. No. | 106 | 107 | 108 | 109 | 110 | 111 | 112 | 113 | 114 | 115 | 116 | 117 | 118 | 119 | 120 |
| Conv No. | 150 | 151 | 152 | 153 | 154 | 155 | 156 | 157 | 158 | 159 | 160 | 161 | 162 | 163 | 164 |
| (SEQ ID 23) | | | | | | | | | | | | | | | |
| Amino Acid | I | N | V | S | S | H | C | P | S | A | G | T | K | C | L |
| Chymo. No. | 121 | 122 | 123 | 124 | 125 | 128 | 129 | 130 | 131 | 132 | 133 | 134 | 135 | 136 | 137 |
| Conv No. | 165 | 166 | 167 | 168 | 169 | 170 | 171 | 172 | 173 | 174 | 175 | 176 | 177 | 178 | 179 |
| (SEQ ID 23) | | | | | | | | | | | | | | | |
| Amino Acid | V | S | G | W | G | T | T | K | S | P | Q | V | H | F | P |
| Chymo. No. | 138 | 139 | 140 | 141 | 142 | 143 | 144 | 145 | 146 | 147 | 148 | 149 | 150 | 151 | 152 |
| Conv No. | 180 | 181 | 182 | 183 | 184 | 185 | 186 | 187 | 188 | 189 | 190 | 191 | 192 | 193 | 194 |
| (SEQ ID 23) | | | | | | | | | | | | | | | |
| Amino Acid | K | V | L | Q | C | L | N | I | S | V | L | S | Q | K | R |
| Chymo. No. | 153 | 154 | 155 | 156 | 157 | 158 | 159 | 160 | 161 | 162 | 163 | 164 | 165 | 166 | 167 |
| Conv No. | 195 | 196 | 197 | 198 | 199 | 200 | 201 | 202 | 203 | 204 | 205 | 206 | 207 | 208 | 209 |
| (SEQ ID 23) | | | | | | | | | | | | | | | |
| Amino Acid | C | E | D | A | Y | P | R | Q | I | D | D | T | M | F | C |
| Chymo. No. | 168 | 169 | 170 | 171 | 172 | 173 | 174 | 174A | 176 | 177 | 178 | 179 | 180 | 181 | 182 |
| Conv No. | 210 | 211 | 212 | 213 | 214 | 215 | 216 | 217 | 218 | 219 | 220 | 221 | 222 | 223 | 224 |
| (SEQ ID 23) | | | | | | | | | | | | | | | |
| Amino Acid | A | G | D | K | A | G | R | D | S | C | Q | G | D | S | G |
| Chymo. No. | 183 | 184 | 185 | 186 | 186A | 187 | 188 | 189 | 190 | 191 | 192 | 193 | 194 | 195 | 196 |
| Conv No. | 225 | 226 | 227 | 228 | 229 | 230 | 231 | 232 | 233 | 234 | 235 | 236 | 237 | 238 | 239 |
| (SEQ ID 23) | | | | | | | | | | | | | | | |
| Amino Acid | G | P | V | V | C | N | G | S | L | Q | G | L | V | S | W |
| Chymo. No. | 197 | 198 | 199 | 200 | 201 | 202 | 203 | 204 | 209 | 210 | 211 | 212 | 213 | 214 | 215 |
| Conv No. | 240 | 241 | 242 | 243 | 244 | 245 | 246 | 247 | 248 | 249 | 250 | 251 | 252 | 253 | 254 |
| (SEQ ID 23) | | | | | | | | | | | | | | | |
| Amino Acid | G | D | Y | P | C | A | R | P | N | R | P | G | V | Y | T |
| Chymo. No. | 216 | 217 | 218 | 219 | 220 | 221 | 221A | 222 | 223 | 224 | 225 | 226 | 227 | 228 | 229 |
| Conv No. | 255 | 256 | 257 | 258 | 259 | 260 | 261 | 262 | 263 | 264 | 265 | 266 | 267 | 268 | 269 |
| (SEQ ID 23) | | | | | | | | | | | | | | | |
| Amino Acid | N | L | C | K | F | T | K | W | I | Q | E | T | I | Q | A |
| Chymo. No. | 230 | 231 | 232 | 233 | 234 | 235 | 236 | 237 | 238 | 239 | 240 | 241 | 242 | 243 | 244 |
| Conv No. | 270 | 271 | | | | | | | | | | | | | |
| (SEQ ID 23) | | | | | | | | | | | | | | | |
| Amino Acid | N | S | | | | | | | | | | | | | |
| Chymo. No. | 245 | 246 | | | | | | | | | | | | | |

It should be understood that the engineered proteases of the disclosure are not limited to those presented in the tables above. Such modifications may increase the half-life, bioavailability, or other characteristics of the serine proteases.

### Fusion Proteins

The engineered proteases can be further modified, e.g. they can include the fusion (addition) of another component or domain. Examples of such components or domains include, but are not limited to, half-life extenders, and activation peptides or activation signals. For example, a component capable of increasing the half-life or bioavailability of an engineered protease of the disclosure can be added. A half-life extender can include, but is not limited to, Fc (e.g., IgG1, IgG2, IgG3, or IgG4), affibodies, PEG, and albumin such as Human Serum Albumin (HSA).

Such additions of the additional components can be added by, for example, PASylation®. Such additions can extend the half-life or bioavailability of the engineered protease compared to a serine protease that does not include the half-life extender. In some embodiments, the addition of a half-life extender or other similar component can also improve or alter any one or more properties of the engineered protease, including, but not limited to, stability, bioavailability, serum half-life, shelf-life, trafficking ability, and immunogenicity.

Accordingly, in some embodiments, the engineered proteases provided herein can further comprise a half-life extender. In some embodiments, the half-life extender is an addition at the N-terminus of the engineered protease. In some embodiments, the half-life extender is an addition at the C-terminus of the engineered protease. In some embodiments, the half-life extender is added directly to the serine protease. In some embodiments, the half-life extender is added to the serine protease via a linker or more than one linker. In some embodiments, the half-life extender is Fc and is a human wild type Fc domain, or a variant thereof. In some embodiments, the half-life extender is albumin, e.g. a human serum albumin, or a variant thereof.

In some embodiments, the engineered proteases provided herein may comprise more than one half-life extender. In some embodiments, each of the half-life extenders are additions at the N-terminus of the serine protease. In some embodiments, each of the half-life extenders are additions at the C-terminus of the serine protease. In some embodiments, one half-life extender is an addition at the N-terminus and the other half-life extender is an addition at the C-terminus of the engineered protease. In some embodiments, the half-life extender is Fc and is a human wild type Fc domain, or a variant thereof. In some embodiments, the half-life extender is albumin, e.g. a human serum albumin, or a variant thereof.

In exemplary embodiments, a chymase-based engineered protease of the disclosure is fused to a wild type Fc domain or variant thereof. In exemplary embodiments, a chymase-based engineered protease of the disclosure is fused to a human serum albumin, or variant thereof.

In exemplary embodiments, a uPA-based engineered protease of the disclosure is fused to a wild type Fc domain or variant thereof. In exemplary embodiments, a uPA-based engineered protease of the disclosure is fused to a human serum albumin, or variant thereof.

In exemplary embodiments, a MTSP-1-based engineered protease of the disclosure is fused to a wild type Fc domain or variant thereof. In exemplary embodiments, a MTSP-1-based engineered protease of the disclosure is fused to a human serum albumin, or variant thereof.

In exemplary embodiments, a KLK5-based engineered protease of the disclosure is fused to a wild type Fc domain or variant thereof. In exemplary embodiments, a KLK5-based engineered protease of the disclosure is fused to a human serum albumin, or variant thereof.

The fusion proteins also can include an activation sequence so that the resulting fusion protein containing an engineered protease of the disclosure is in an active form, such as a two chain form. Activation sequences can contain or be modified to contain a cysteine, which can form a disulfide bond with a free Cys, such as C122, for example, in the modified u-PA polypeptide, whereby, upon activation, the resulting activated polypeptide comprises two chains. Exemplary activation sequences include a enterokinase activation sequence and a furin activation sequence, and modified forms thereof.

### Activity of Engineered Proteases

In some embodiments, the engineered proteases of the disclosure cleave Factor B at a site not targeted by Factor D, or at a site targeted by Factor D, and the cleavage at such site results in a reduction of a function of Factor B or a Factor B fragment. In some embodiments, the function of Factor B or a Factor B fragment is an interaction with at least one complement component. In some embodiments, the function of Factor B or a Factor B fragment is an interaction with hydrolyzed soluble C3. In some embodiments, the function of Factor B or a Factor B fragment is an interaction with C3b. In some embodiments, the C3b is a membrane-bound C3b. In some embodiments, cleavage at a non-Factor D site occurs when Factor B is not bound to C3b. In some embodiments, cleavage at a Factor D site occurs when Factor B is not bound to C3b. In some embodiments, cleavage at a non-Factor D site occurs when Factor B is bound to C3b (i.e., complexed with C3b).

In some embodiments, the engineered proteases provided herein can cleave other peptide substrates that are not Factor B, while also being capable of cleaving Factor B. In some embodiments, the cleavage activity for a non-Factor B peptide substrate is about equal to or less than cleavage activity for the Factor B site.

In some embodiments, the engineered proteases provided herein have a K_{cat}/Kₘ of about 100, about 200, about 300, about 400, about 500, about 600, about 700, about 800, about 900, about 1,000, about 1,000, about 1,100, about 1,200, about 1,300, about 1,400, about 1,500, about 1,600, about 1,700, about 1,800, or about 1,900 M⁻¹ s⁻¹ for Factor B cleavage. In some embodiments, the K_{cat}/Kₘ is up to or greater than about 10e⁸. In some embodiments, the engineered proteases provided herein have a K_{cat}/Kₘ of about 10³ to about 10⁶ M⁻¹ s⁻¹ for Factor B cleavage.

In some embodiments, the engineered proteases provided herein have an EC₅₀ for Factor B cleavage of about 1 nM to about 20 nM. In some embodiments, the engineered proteases provided herein have an EC₅₀ for Factor B of less than about 20 nM. In some embodiments, the engineered proteases provided herein have an EC₅₀ for Factor B cleavage of less than about 1 nM. In some embodiments, the engineered proteases provided herein have an EC₅₀ for Factor B cleavage of about 5 nM to about 100 nM. In some embodiments, the engineered proteases provided herein have an EC₅₀ for cleavage of Factor B of about 20, about 25, or about 60 nM. In some embodiments, the EC₅₀ for cleavage of Factor B is about 20 nM. In some embodiments, the EC₅₀ for Factor B cleavage is about 50 nM. In some embodiments, the engineered proteases provided herein have an EC₅₀ for Factor B cleavage of about 1,000 nM to about 4,500 nM. In some embodiments, the engineered proteases provided herein have an EC₅₀ for Factor B cleavage of about 1,000 nM, or about 2,000 nM, or about 3,000 nM, or about 4,000 nM, or about 5,000 nM.

In some embodiments, the engineered proteases provided herein have a catalytic lifetime in human plasma of over about 72 hours. In some embodiments, the engineered proteases provided herein have a catalytic lifetime in human plasma of about or greater than about 120 hours. In some embodiments, the engineered proteases provided herein have a catalytic lifetime in human plasma of about 120 hours or more and are useful for chronic indications. In some embodiments, the engineered proteases provided herein have a catalytic lifetime in human plasma of about 24 hours. In some embodiments, the engineered proteases provided herein have a catalytic lifetime in human plasma of about 24 hours or more and are useful for acute indications.

In some embodiments, the engineered proteases provided herein have catalytic activity for about 1 day, about 2 days, about 3 days, about 4 days, about 5 days, about 6 days, about 7 days, about 8 days, about 9 days, or about 10 days. In some embodiments, the catalytic activity is between about 10% and about 85% of the initially measured activity after about 7 days.

In some embodiments, the engineered proteases provided herein have an increased half-life compared to an MTSP-1 or MTSP-1 protease domain that is not modified. In some embodiments, the engineered proteases provided herein have an increased bioavailability compared to an MTSP-1 or MTSP-1 protease domain that is not modified. In some embodiments, the engineered proteases provided herein have an increased half-life compared to a uPA or uPA protease domain that is not modified. In some embodiments, the engineered proteases provided herein have an increased bioavailability compared to a uPA or uPA protease domain that is not modified. In some embodiments, the engineered proteases provided herein have an increased half-life compared to a chymase or chymase protease domain that is not modified. In some embodiments, the engineered proteases provided herein have an increased bioavailability compared to a chymase or chymase protease domain that is not modified. In some embodiments, the engineered proteases provided herein have an increased half-life compared to a KLK5 or KLK5 protease domain that is not modified. In some embodiments, the engineered proteases provided herein have an increased bioavailability compared to a KLK5 or KLK5 protease domain that is not modified.

In some embodiments, the engineered proteases provided herein are non-immunogenic.

In some embodiments, the engineered proteases provided herein are in a zymogen form. As used herein, the zymogen form refers to a full-length protease prior to cleavage into the mature form. In some embodiments, the engineered proteases provided herein are in an active form, also referred to as the mature form. In some embodiments, the zymogen form may be activated to the mature form in vivo (in situ) upon administration. In some embodiments the zymogen form is activated ex vivo (in vitro) prior to e.g. administration of the engineered protease.

In some embodiments, the engineered protease is in an activated form. In some embodiments, the engineered protease is activated by an enzyme, e.g. an enterokinase. In some embodiments, a chymase-based engineered protease of the disclosure is activated by an enterokinase. In some embodiments, the engineered protease is activated during recombinant production in a host cell. In some embodiments, the activation by an enzyme during production in a host cell is by overexpression of the enzyme, e.g. an enterokinase. In some embodiments, the engineered protease is activated after production and secretion by a host cell, optionally in the media.

### Uses of Engineered Proteases

The engineered proteases of the disclosure may be used for modulating the complement system.

In some embodiments, the engineered proteases of the disclosure are capable of modulating the classical complement pathway. In some embodiments, the engineered proteases of the disclosure are capable of modulating the alternate complement pathway. In some embodiments, the engineered proteases of the disclosure are capable of modulating the lectin complement pathway. In some embodiments, the engineered proteases of the disclosure are capable of decreasing the amplification of the complement system.

In some embodiments, the engineered proteases of the disclosure are capable of reducing a function of Factor B or a Factor B fragment. As discussed herein, in some embodiments, the engineered proteases of the disclosure are capable of reducing generation of Factor B fragments Ba and/or Bb or producing Factor B fragments Ba and/or Bb that are functionally inactive.

Provided herein is a method of inactivating Factor B, comprising contacting the Factor B with any of the engineered proteases disclosed herein. In some embodiments, using such a method, complement activation is inhibited. In some embodiments, the classical pathway of the complement pathway is inhibited. In some embodiments, the alternate pathway of the complement pathway is inhibited. In some embodiments, the lectin pathway of the complement pathway is inhibited.

In some embodiments, the method is in vitro. In some embodiments, the method is in vivo.

The engineered proteases of the disclosure may be used for therapeutics in a subject. Accordingly, provided herein is a method of treating a disease or condition in a subject in need thereof, comprising administering to the subject any one of the engineered proteases of the disclosure. In some embodiments, the disease or condition is associated with dysregulated complement, accordingly, in some embodiments, the disease or condition involves complement dysregulation. In some embodiments, the treatment is a replacement therapy. In some embodiments, the treatment blocks complement activation. In some embodiments, the treatment modulates autoimmunity. In some embodiments, the treatment is for endothelial or kidney cell injury.

In some exemplary embodiments, the disease or condition is selected from lupus nephritis, C3 glomerulopathy (C3G), primary IgA nephropathy, kidney transplant ischemia and reperfusion (I/R) injury, antineutrophil cytoplasmic antibody (ANCA)-associated vasculitis (AAV), sepsis, acute respiratory distress syndrome (ARDS), SARS-associated coronavirus (SARS-CoV), atypical hemolytic uremic syndrome (aHUS), membranous nephropathy (MN) and paroxysmal nocturnal hemoglobinuria (PNH).

In some embodiments, the engineered proteases provided herein are useful for treatment of inflammatory diseases or condition. In some embodiments, the engineered proteases provided herein are capable of reducing inflammatory cytokines. In some exemplary embodiments, the engineered proteases provided herein are efficacious in reducing inflammatory cytokines IL-2 and IL-6, and chemokine CXCL9 and are useful for the treatment of diseases such as ARDS.

In some embodiments, the engineered protease is administered to the subject subcutaneously. In some embodiments, the engineered protease is activated in situ at the site of a dysregulated complement component, or at the site of dysregulated pathophysiology. In some embodiments, the engineered protease is provided in a liquid stable formulation. The in vivo administration of the engineered proteases can be carried out intravenously, intramuscularly, subcutaneously, topically, orally, transdermally, intraperitoneally, intraorbitally, intrathecally, intraarterially, intraventricularly, intranasally, transmucosally, through implantation, or through inhalation. In some embodiments, the engineered proteases provided herein are administered with a mechanical device.

### Pharmaceutical Compositions

The disclosure also provides pharmaceutical compositions comprising any one of the engineered proteases disclosed herein, and optionally a pharmaceutical acceptable excipient or carrier. In some embodiments, the pharmaceutical composition is sterile. The pharmaceutical compositions may be formulated to be compatible with their intended routes of administration. In some embodiments, the pharmaceutical compositions of the disclosure are suitable for administration to a human subject, or other non-human primate.

### Kits and Articles of Manufacture

The disclosure also provides a kit or article of manufacture comprising any one of the engineered proteases disclosed herein, or any pharmaceutical composition disclosed herein. In some embodiments, the kits may further include instructional materials for carrying out any of the methods disclosed herein. In some embodiments, the kits may further include sterile containers or vials for holding the fusion constructs and/or pharmaceutical compositions disclosed herein. In some embodiments, the kits may further include sterile delivery devices for administering the fusion constructs and/or pharmaceutical compositions disclosed herein. In some embodiments, an article of manufacture comprises any pharmaceutical composition of the disclosure.

### Production of Engineered Proteases

Provided herein are methods and compositions for generating engineered proteases. Accordingly, provided herein are nucleic acids and vectors encoding any of the engineered proteases of the disclosure. Also provided are cells comprising one or more nucleic acids encoding an engineered protease of the disclosure.

The engineered proteases provided herein can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Provided herein are methods of generating an engineered protease of the disclosure using an expression system, e.g. the engineered protease of the disclosure may be expressed in bacterial (e.g. E. coli), yeast, insect, or mammalian cells (e.g. CHO cells, HEK cells). In specific embodiments, transformation of host cells with recombinant DNA molecules that incorporate the isolated an engineered protease protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA. In some embodiments, the engineered protease is active upon production. In some embodiments, the engineered protease needs to be activated upon production. In such embodiments, an engineered protease is engineered in an activated state; the method comprising producing the engineered protease in a zymogen form in a bacterial, yeast, or mammalian host system, and subsequently activated.

### Administration of Engineered Proteases

In some embodiments, provided herein are methods for administering an engineered protease of the disclosure by delivery of vectors/nucleic acids encoding the engineered protease. In some embodiments, the methods involve administration of recombinant vectors. In some embodiments, provided herein are engineered proteases for use in gene expression therapy using non-viral vectors. In other embodiments, provided herein are engineered proteases for use in gene expression therapy using viral vectors. In some embodiments, cells are engineered to express an engineered protease, such as by integrating an engineered protease encoding-nucleic acid into a genomic location, either operatively linked to regulatory sequences or such that it is placed operatively linked to regulatory sequences in a genomic location. In some embodiments, such cells are then administered locally or systemically to a subject, such as a subject in need of treatment.

Methods for amplification of nucleic acids can be used to isolate nucleic acid molecules encoding an engineered protease, including for example, polymerase chain reaction (PCR) methods. A nucleic acid containing material can be used as a starting material from which an engineered protease-encoding nucleic acid molecule can be isolated. For example, DNA and mRNA preparations, cell extracts, tissue extracts (e.g. from liver), fluid samples (e.g. blood, serum, saliva), samples from healthy and/or diseased subjects can be used in amplification methods. Nucleic acid libraries also can be used as a source of starting material. Primers can be designed to amplify and modify an engineered protease-encoding molecule. For example, primers can be designed based on expressed sequences from which an engineered protease is generated.

Additional nucleotide sequences can be joined to an engineered protease-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to an engineered protease-encoding nucleic acid molecule. Examples of such sequences include, but are not limited to, promoter sequences designed to facilitate intracellular protein expression, and secretion sequences designed to facilitate protein secretion. Additional nucleotide sequences such as sequences specifying protein binding regions also can be linked to an engineered protease-encoding nucleic acid molecules. Such regions include, but are not limited to, sequences to facilitate uptake of an engineered protease into specific target cells, or otherwise enhance the pharmacokinetics of the synthetic gene.

### Enumerated Embodiments

The disclosure provides for the following sets of non-limiting enumerated embodiments.

### Set I

Embodiment I-1. An engineered protease of the S1A serine protease family, wherein the engineered protease is specific for and is capable of cleaving Factor B.

Embodiment I-2. The engineered protease of embodiment I-1, wherein cleavage of Factor B by the engineered protease generates one or more functionally inactive fragments.

Embodiment I-3. The engineered protease of any of embodiment I-2, wherein the one or more functionally inactive fragments are capable of reducing complement activation.

Embodiment I-4. The engineered protease of any one of embodiments I-3, wherein cleavage of Factor B results in the generation of a Factor B fragment that is reduced in function or results in a Factor B that is reduced in function.

Embodiment I-5. The engineered protease of any one of embodiments I-4, wherein the Factor B is a rodent Factor B.

Embodiment I-6. The engineered protease of any one of embodiments I-4, wherein the Factor B is a non-human primate Factor B.

Embodiment I-7. The engineered protease of embodiment I-6, wherein the non-human primate is cynomolgus monkey.

Embodiment I-8. The engineered protease of any one of embodiments I-4, wherein the Factor B is human Factor B.

Embodiment I-9. The engineered protease of embodiment I-8, wherein the Factor B comprises the amino acid sequence as set forth in SEQ ID NO: 1.

Embodiment I-10. The engineered protease of any one of embodiments I-9, wherein cleavage of Factor B occurs at a site not targeted by Factor D.

Embodiment I-11. The engineered protease of embodiment I-10, wherein cleavage at the site generates at least two fragments that are not Ba and Bb.

Embodiment I-12. The engineered protease of any one of embodiments I-11, wherein cleavage at the site results in a reduction of the generation of Factor B cleavage products Ba and Bb as compared to cleavage by Factor D.

Embodiment I-13. The engineered protease of any one of embodiments I-9, wherein cleavage of Factor B occurs at a site that is targeted by Factor D.

Embodiment I-14. The engineered protease of any one of embodiments I-13, wherein the site targeted by Factor D comprises QQKR/KIV (SEQ ID NO: 9).

Embodiment I-15. The engineered protease of embodiment I-10, wherein the Factor B cleavage site comprises a sequence selected from: WEHR/KGT (SEQ ID NO: 10), KNQKR/QKQ (SEQ ID NO: 11), DVFY/QMI (SEQ ID NO: 12), EGVD/AE (SEQ ID NO: 13), DHKL/KSG (SEQ ID NO: 14), TPW/SLA (SEQ ID NO: 15), KVSEAD (SEQ ID NO: 20), IRPSKG (SEQ ID NO: 4), GGEKRD (SEQ ID NO: 5), GKKEAG (SEQ ID NO: 3), and DHKL/KSG (SEQ ID NO: 21).

Embodiment I-16. The engineered protease of any one of embodiments I-15, wherein the engineered protease is based on a chymotrypsin-like serine protease selected from the group consisting of: membrane type serine protease 1 (MTSP-1), urokinase-type plasminogen activator (uPA), chymase, and Kallikrein-related peptidase 5 (KLK5).

Embodiment I-17. The engineered protease of embodiment I-16, wherein the engineering of the engineered protease involves one or more modifications selected from the group consisting of substitution, addition, and deletion of an amino acid residue, and substitution, addition, and deletion of a domain of the chymotrypsin-like serine protease.

Embodiment I-18. The engineered protease of any one of embodiments 16-17, wherein the engineered protease is based on MTSP-1 or uPA, and the cleavage site comprises a sequence selected from: WEHR/KGT (SEQ ID NO: 10) and KNQKR/QKQ (SEQ ID NO: 11).

Embodiment I-19. The engineered protease of any one of embodiments I-18, wherein the engineered protease is based on a MTSP-1.

Embodiment I-20. The engineered protease of any one of embodiments I-18, wherein the engineered protease is not based on a MTSP-1.

Embodiment I-21. The engineered protease of embodiment I-19, comprising one or more modifications with respect to a MTSP-1 comprising an amino acid sequence as set forth in SEQ ID NO: 7, wherein the residues are numbered by chymotrypsin numbering.

Embodiment I-22. The engineered protease of any one of embodiments I-18, wherein the engineered protease is based on a uPA.

Embodiment I-23. The engineered protease of any one of embodiments I-18, wherein the engineered protease is not based on a uPA.

Embodiment I-24. The engineered protease of embodiment I-22, comprising one or more modifications with respect to a uPA comprising an amino acid sequence as set forth in SEQ ID NO: 8, wherein the residues are numbered by chymotrypsin numbering.

Embodiment I-25. The engineered protease of any one of embodiments I-17, wherein the engineered protease is based on chymase.

Embodiment I-26. The engineered protease of embodiment I-25, wherein the engineered protease is based on chymase, and the cleavage site comprises a sequence selected from DVFY/QMI (SEQ ID NO: 12), EGVD/AE (SEQ ID NO: 13), DHKL/KSG (SEQ ID NO: 14), and TPW/SLA (SEQ ID NO: 15).

Embodiment I-27. The engineered protease of any one of embodiments I-1-17, wherein the engineered protease is based on KLK5.

Embodiment I-28. The engineered protease of embodiment I-25, comprising one or more modifications with respect to a chymase comprising an amino acid sequence as set forth in SEQ ID NO: 6, wherein the residues are numbered by chymotrypsin numbering.

Embodiment I-29. The engineered protease of embodiment I-19, wherein the one or more modifications is at one or more positions corresponding to one or more positions selected from D23, 141, L70, A77, F94, D96, F97, T98, F99, K110, C122, D125, Y146, Q175, V183, Q192, A204, D217, and K224 in a MTSP-1 comprising the sequence of amino acids set forth in SEQ ID NO: 7, wherein the residues are numbered by chymotrypsin numbering.

Embodiment I-30. The engineered protease of embodiment I-22, wherein the one or more modifications is at one or more positions corresponding to one or more positions selected from G18, R36, S37, V38, Y40, D60, A96, L97, A98, H99, C122, Y151, V159, A184, Q192, R217, K224 in a uPA comprising the sequence of amino acids set forth SEQ ID NO: 8, wherein the residues are numbered by chymotrypsin numbering.

Embodiment I-31. The engineered protease of embodiment I-25, wherein the one or more modifications is one or more positions corresponding to one or more positions selected from C22, S36, P38, G43, R49, K87, K93, 1103, L114, L116, F123, V138, F173, D175, 5189, A190, F191, K192, L199, V213, G216, A220, A226, F228 in a chymase comprising the sequence of amino acids set forth in SEQ ID NO: 6, wherein the residues are numbered by chymotrypsin numbering.

Embodiment I-32. The engineered protease of any one of embodiments 4-31, wherein the function of Factor B or a Factor B fragment is an interaction with at least one complement component.

Embodiment I-33. The engineered protease of any one of embodiments 4-31, wherein the function of Factor B or a Factor B fragment is an interaction with hydrolyzed soluble C3.

Embodiment I-34. The engineered protease of any one of embodiments 4-33, wherein the function of Factor B or a Factor B fragment is an interaction with C3b.

Embodiment I-35. The engineered protease of any one of embodiments 4-34, wherein the function of Factor B or a Factor B fragment is an interaction with membrane-bound C3b.

Embodiment I-36. The engineered protease of any one of embodiments I-35, wherein cleavage occurs when Factor B is not bound to C3b.

Embodiment I-37. The engineered protease of any one of embodiments I-36, wherein the cleavage activity for a non-Factor B peptide substrate is about equal to or less than cleavage activity for the Factor B site.

Embodiment I-38. The engineered protease of any one of embodiments I-36, wherein the engineered protease has a kcat/Km of about 100, about 200, about 300, about 400, about 500, about 600, about 700, about 800, about 900, about 1,000, about 1,000, about 1,100, about 1,200, about 1,300, about 1,400, about 1,500, about 1,600, about 1.700, about 1,800, or about 1,900 M-1 s-1 for Factor B cleavage.

Embodiment I-39. The engineered protease of any one of embodiments I-38, wherein the engineered protease has a kcat/Km of about 103 to about 109 M-1 s-1 for Factor B cleavage.

Embodiment I-40. The engineered protease of any one of embodiments I-39, wherein the engineered protease has an EC50 for Factor B of less than about 20 nM.

Embodiment I-41. The engineered protease of any one of embodiments I-40, wherein the engineered protease has an EC50 for Factor B of less than about 1 nM.

Embodiment I-42. The engineered protease of any one of embodiments I-39, wherein the engineered protease has an EC50 for Factor B of about 20, about 25, or about 60 nM.

Embodiment I-43. The engineered protease of any one of embodiments I-39, wherein the engineered protease has an EC50 for cleaving Factor B of about 1,000 to about 4,500 nM.

Embodiment I-44. The engineered protease of any one of embodiments I-43, wherein the engineered protease has a plasma half-life in human plasma of over about 72 hours.

Embodiment I-45. The engineered protease of any one of embodiments I-44, wherein the engineered protease has a plasma half-life in human plasma of over about 120 hours.

Embodiment I-46. The engineered protease of any one of embodiments I-45, wherein the engineered protease has a plasma half-life in human plasma of about 7 days.

Embodiment I-47. The engineered protease of embodiment I-46, wherein the catalytic activity is about 10% to about 50%, or about 90% to about 100%.

Embodiment I-48. The engineered protease of Embodiment I-29, wherein the engineered protease has an increased half-life compared to an MTSP-1 that is not modified.

Embodiment I-49. The engineered protease of any one of embodiments I-29 and I-48, wherein the engineered protease has an increased bioavailability compared to an MTSP-1 that is not modified.

Embodiment I-50. The engineered protease of Embodiment I-30, wherein the engineered protease has an increased half-life compared to a uPA that is not modified.

Embodiment I-51. The engineered protease of any one of embodiments I-30 and I-50, wherein the engineered protease has an increased bioavailability compared to a uPA that is not modified.

Embodiment I-52. The engineered protease of Embodiment I-31, wherein the engineered protease has an increased half-life compared to a chymase that is not modified.

Embodiment I-53. The engineered protease of any one of embodiments I-31 and I-52, wherein the engineered protease has an increased bioavailability compared to a chymase that is not modified.

Embodiment I-54. The engineered protease of any one of embodiments I-1 to I-53, wherein the engineered protease is non-immunogenic.

Embodiment I-55. The engineered protease of any one of embodiments Set I 1-54, wherein the engineered protease is in a zymogen form.

Embodiment I-56. The engineered protease of any one of embodiments Set I 1-54, wherein the engineered protease is in an active form.

Embodiment I-57. The engineered protease of any one of embodiments Set I 1-56, further comprising a half-life extender.

Embodiment I-58. A method of inactivating Factor B, comprising contacting the Factor B with any of the engineered proteases of embodiments I-1 to I-57.

Embodiment I-59. The method of Embodiment I-58, wherein complement activation is inhibited.

Embodiment I-60. The method of Embodiment I-59, wherein the classical pathway of the complement pathway is inhibited.

Embodiment I-61. The method of any one of embodiments 59-60, wherein the alternate pathway of the complement pathway is inhibited.

Embodiment I-62. The method of any one of embodiments 59-61, wherein the lectin pathway of the complement pathway is inhibited.

Embodiment I-63. The method of any one of embodiments set I 58-62, wherein the method is in vitro.

Embodiment I-64. The method of any one of embodiments Set I 58-62, wherein the method is in vivo.

Embodiment I-65. A method of treating a disease or condition in a subject in need thereof, comprising administering to the subject any one of the engineered proteases of embodiments I-57.

Embodiment I-66. The method of Embodiment I-65, wherein the disease or condition is associated with dysregulated complement.

Embodiment I-67. The method of any one of embodiments Set I 65-66, wherein the disease or condition is an inflammatory disease or condition.

Embodiment I-68. The method of any one of embodiments 65-67, wherein the treatment is a replacement therapy.

Embodiment I-69. The method of any one of embodiments Set I 65-68, wherein the treatment blocks complement activation.

Embodiment I-70. The method of any one of embodiments Set 165-69, wherein the treatment modulates autoimmunity.

Embodiment I-71. The method of any one of embodiments Set I 65-70, wherein the disease or condition is a congenital complement deficiency.

Embodiment I-72. The method of any one of embodiments Set I 65-71, wherein the treatment is for endothelial or kidney cell injury.

Embodiment I-73. The method of any one of embodiments Set I 65-72, wherein the disease or condition is selected from lupus nephritis, C3 glomerulopathy (C3G), primary IgA nephropathy, kidney transplant ischemia and reperfusion (I/R) injury, antineutrophil cytoplasmic antibody (ANCA)-associated vasculitis (AAV), sepsis, acute respiratory distress syndrome (ARDS), SARS-associated coronavirus (SARS-CoV), atypical hemolytic uremic syndrome (aHUS), membranous nephropathy (MN) and paroxysmal nocturnal hemoglobinuria (PNH).

Embodiment I-74. The method of any one of embodiments Set I 65-73, wherein the disease or condition is a control protein deficiency.

Embodiment I-75. The method of any one of embodiments Set I 65-73, wherein the disease or condition is a secondary complement disorder.

Embodiment I-76. The method of any one of embodiments Set I 65-73, wherein the disease or condition is an immunity-related disease or condition.

Embodiment I-77. The method of any one of embodiments Set I 65-76, wherein the engineered protease is administered to the subject subcutaneously.

Embodiment I-78. The method of Embodiment I-77, wherein the engineered protease is activated in situ at the site of a dysregulated complement component.

Embodiment I-79. The method of any of any one of embodiments Set I 65-78, wherein the engineered protease is provided in a liquid stable formulation.

Embodiment I-80. A pharmaceutical composition comprising any of the engineered proteases of embodiments Set I 1-57, and optionally a pharmaceutically acceptable carrier.

Embodiment I-81. The pharmaceutical composition of Embodiment I-80, wherein the engineered protease is provided in a liquid stable formulation.

Embodiment I-82. The pharmaceutical composition of any one of embodiments Set I 80-81, wherein the composition is formulated for subcutaneous administration.

### Set II

Embodiment II-1. An engineered protease comprising a modified chymase protease domain, a modified membrane type serine protease 1 (MTSP-1) protease domain, a modified urokinase-type plasminogen activator (uPA) protease domain, or a modified Kallikrein-related peptidase 5 (KLK5) protease domain, wherein the engineered protease is capable of cleaving Factor B.

Embodiment 11-2. The engineered protease of embodiment II-1, wherein cleavage of Factor B by the engineered protease generates one or more functionally inactive fragments.

Embodiment 11-3. The engineered protease of embodiment 11-2, wherein the one or more functionally inactive fragments are capable of reducing complement activation.

Embodiment 11-4. The engineered protease of any one of embodiments II-1 to 11-3, wherein cleavage of Factor B results in the generation of a Factor B fragment that is reduced in function.

Embodiment 11-5. The engineered protease of any one of embodiments II-1 to 11-4, wherein the Factor B is a non-human primate Factor B.

Embodiment 11-6. The engineered protease of embodiment 11-5, wherein the non-human primate is cynomolgus monkey.

Embodiment 11-7. The engineered protease of any one of embodiments II-1 to 11-4, wherein the Factor B is human Factor B.

Embodiment 11-8. The engineered protease of embodiment 11-7, wherein the Factor B comprises the amino acid sequence as set forth in SEQ ID NO: 1.

Embodiment 11-9. The engineered protease of any one of embodiments II-1 to 11-8, wherein cleavage of Factor B occurs at a site not targeted by Factor D.

Embodiment II-10. The engineered protease of embodiment 11-9, wherein cleavage at the site not targeted by Factor D generates at least two fragments that are not Ba and Bb.

Embodiment II-11. The engineered protease of any one of embodiments II-1 to II-10, wherein cleavage of Factor B results in a reduction of the generation of Factor B cleavage products Ba and Bb as compared to cleavage by Factor D.

Embodiment 11-12. The engineered protease of any one of embodiments II-1 to 11-8, wherein cleavage of Factor B occurs at a site that is targeted by Factor D.

Embodiment 11-13. The engineered protease of embodiment 11-12, wherein the Factor B cleavage site targeted by Factor D comprises QQKR/KIV (SEQ ID NO: 9).

Embodiment 11-14. The engineered protease of embodiment 11-9, wherein the Factor B cleavage site comprises a sequence selected from: WEHR/KGT (SEQ ID NO: 10), KNQKR/QKQ (SEQ ID NO: 11), DVFY/QMI (SEQ ID NO: 12), EGVD/AE (SEQ ID NO: 13), DHKL/KSG (SEQ ID NO: 14), TPW/SLA (SEQ ID NO: 15), KVSEAD (SEQ ID NO: 20), IRPSKG (SEQ ID NO: 4), GGEKRD (SEQ ID NO: 5), GKKEAG (SEQ ID NO: 3), and DHKL/KSG (SEQ ID NO: 21).

Embodiment II-15. The engineered protease of embodiment 11-9, wherein the Factor B cleavage site comprises a sequence selected from WEHR/KGT (SEQ ID NO: 10) and KNQKR/QKQ (SEQ ID NO: 11), and wherein the engineered protease comprises a modified MTSP-1 protease domain or a modified uPA protease domain.

Embodiment 11-16. The engineered protease of any one of embodiments II-1 to 11-15, wherein the engineered protease comprises a modified MTSP-1 protease domain.

Embodiment 11-17. The engineered protease of any one of embodiments II-1 to 11-15, wherein the engineered protease does not comprise a modified MTSP-1 protease domain.

Embodiment 11-18. The engineered protease of embodiment 11-16, comprising one or more modifications with respect to a MTSP-1 protease domain comprising an amino acid sequence as set forth in SEQ ID NO: 7.

Embodiment 11-19. The engineered protease of embodiment 11-18, wherein the modification is one or more of a substitution, an addition, and deletion of one or more amino acid residues.

Embodiment 11-20. The engineered protease of embodiment 11-16, wherein the one or more modifications is at one or more positions corresponding to one or more positions selected from D622, 1640, L678, A686, F703, D705, F706, T707, F708, K719, C731, D734, Y755, Q783, V791, Q802, A814, D828, and K835 in a MTSP-1 protease domain comprising the sequence of amino acids set forth in SEQ ID NO: 18.

Embodiment 11-21. The engineered protease of embodiment 11-16, wherein the one or more modifications are selected from those presented in Table 5A.

Embodiment 11-22. The engineered protease of embodiment 11-16, wherein the one or more modifications are selected from those exemplary mutation strings presented in Table 5B.

Embodiment 11-23. The engineered protease of any one of embodiments II-1 to 11-15, wherein the engineered protease comprises a modified uPA protease domain.

Embodiment 11-24. The engineered protease of any one of embodiments II-1 to 11-15, wherein the engineered protease does not comprise a modified uPA protease domain.

Embodiment 11-25. The engineered protease of embodiment 11-23, comprising one or more modifications with respect to a uPA protease domain comprising an amino acid sequence as set forth in SEQ ID NO: 8.

Embodiment 11-26. The engineered protease of embodiment 11-25, wherein the modification is one or more of a substitution, an addition, and deletion of one or more amino acid residues.

Embodiment 11-27. The engineered protease of embodiment 11-23, wherein the one or more modifications is at one or more positions corresponding to one or more positions selected from S37, D60, L97, G161, R179, H180, V185, Y187, 1207, A247, D248, A251, H252, C279, Y308, V316, A343, Q353, R378, K385 in a uPA protease domain comprising the sequence of amino acids set forth SEQ ID NO: 8.

Embodiment 11-28. The engineered protease of embodiment 11-23, wherein the one or more modifications are selected from those presented in Table 3A.

Embodiment 11-29. The engineered protease of embodiment 11-23, wherein the one or more modifications are selected from those exemplary mutation strings presented in Table 3B.

Embodiment 11-30. The engineered protease of any one of embodiments II-1 to 11-19, wherein the engineered protease comprises a modified chymase protease domain.

Embodiment 11-31. The engineered protease of any one of embodiments II-1 to 11-19, wherein the engineered protease does not comprise a modified chymase protease domain.

Embodiment 11-32. The engineered protease of embodiment 11-30, wherein the engineered protease comprises a modified chymase protease domain, and the cleavage site comprises a sequence selected from DVFY/QMI (SEQ ID NO: 12), EGVD/AE (SEQ ID NO: 13), DHKL/KSG (SEQ ID NO: 14), and TPW/SLA (SEQ ID NO: 15).

Embodiment 11-33. The engineered protease of embodiment 11-30, comprising one or more modifications with respect to a chymase protease domain comprising an amino acid sequence as set forth in SEQ ID NO: 6.

Embodiment 11-34. The engineered protease of embodiment 11-33, wherein the modification is one or more of a substitution, an addition, and deletion of one or more amino acid residues.

Embodiment 11-35. The engineered protease of embodiment 11-30, wherein the one or more modifications is one or more positions corresponding to one or more positions selected from S36 C7, V21, P26, G31, R37, K74, K80, 190, L101, L103, F110, V125, F157, D159, 5176, A177, F178, K179, L186, V196, G199, A203, A207, F209 in a chymase protease domain comprising the sequence of amino acids set forth in SEQ ID NO: 6.

Embodiment 11-36. The engineered protease of embodiment 11-30, wherein the one or more modifications are selected from those presented in Table 7A.

Embodiment 11-37. The engineered protease of embodiment 11-30, wherein the one or more modifications are selected from those exemplary mutation strings presented in Table 7B.

Embodiment 11-38. The engineered protease of any one of embodiments II-1 to 11-19, wherein the engineered protease comprises a modified KLK5 protease domain, optionally comprising one or more amino acid modifications of SEQ ID NO: 23.

Embodiment 11-39. The engineered protease of any one of embodiments II-1 to 11-19, wherein the engineered protease does not comprise a modified KLK5 protease domain.

Embodiment 11-40. The engineered protease of any one of embodiments II-1 to 11-39, wherein the engineered protease has a k_{cat}/Kₘ of about 100, about 200, about 300, about 400, about 500, about 600, about 700, about 800, about 900, about 1,000, about 1,000, about 1,100, about 1,200, about 1,300, about 1,400, about 1,500, about 1,600, about 1.700, about 1,800, or about 1,900 M⁻¹ s⁻¹ for Factor B cleavage.

Embodiment 11-41. The engineered protease of any one of embodiments II-1 to 11-40, wherein the engineered protease has a k_{cat}/Kₘ of about 10³ to about 10⁹ M⁻¹ s⁻¹ for Factor B cleavage.

Embodiment 11-42. The engineered protease of any one of embodiments II-1 to 11-41, wherein the engineered protease has an EC₅₀ for Factor B of less than about 20 nM.

Embodiment 11-43. The engineered protease of any one of embodiments II-1 to 11-42, wherein the engineered protease has an EC₅₀ for Factor B of less than about 1 nM.

Embodiment 11-44. The engineered protease of any one of embodiments II-1 to 11-41, wherein the engineered protease has an EC₅₀ for Factor B of about 20, about 25, or about 60 nM.

Embodiment 11-45. The engineered protease of any one of embodiments II-1 to 11-41, wherein the engineered protease has an EC₅₀ for cleaving Factor B of about 1,000 to about 4,500 nM.

Embodiment 11-46. The engineered protease of any one of embodiments II-1 to 11-45, wherein the engineered protease has a plasma half-life in human plasma of over about 72 hours.

Embodiment 11-47. The engineered protease of any one of embodiments II-1 to 11-46, wherein the engineered protease has a plasma half-life in human plasma of over about 120 hours.

Embodiment 11-48. The engineered protease of any one of embodiments II-1 to 11-47, wherein the engineered protease has a plasma half-life in human plasma of about 7 days.

Embodiment 11-49. The engineered protease of embodiment 11-48, wherein the catalytic activity is about 10% to about 50%, or about 90% to about 100%.

Embodiment 11-50. The engineered protease of embodiment 11-16, wherein the engineered protease has an increased half-life compared to protease comprising a MTSP-1 protease domain that is not modified.

Embodiment 11-51. The engineered protease of embodiment 11-16, wherein the engineered protease has an increased bioavailability compared to an protease comprising a MTSP-1 protease domain that is not modified.

Embodiment 11-52. The engineered protease of embodiment 11-23, wherein the engineered protease has an increased half-life compared to a protease comprising a uPA protease domain that is not modified.

Embodiment 11-53. The engineered protease of embodiment 11-23, wherein the engineered protease has an increased bioavailability compared to a protease comprising a uPA protease domain that is not modified.

Embodiment 11-54. The engineered protease of embodiment 11-30, wherein the engineered protease has an increased half-life compared to protease comprising a chymase protease domain that is not modified.

Embodiment 11-55. The engineered protease of embodiment 11-30, wherein the engineered protease has an increased bioavailability compared to protease comprising a chymase protease domain that is not modified.

Embodiment 11-56. The engineered protease of any one of embodiments II-1 to 11-55, wherein the engineered protease is non-immunogenic.

Embodiment 11-57. The engineered protease of any one of embodiments II-1 to 11-56, wherein the engineered protease is in a zymogen form.

Embodiment II-58. The engineered protease of any one of embodiments II-1 to 11-56, wherein the engineered protease is in an active form.

Embodiment 11-59. The engineered protease of any one of embodiments II-1 to II-58, wherein the engineered protease is fused to a component that extends the half-life of the engineered protease.

Embodiment 11-60. The engineered protease of embodiment II-59, wherein the component is a Fc domain.

Embodiment 11-61. The engineered protease of embodiment 11-59, wherein the component is a human serum albumin.

Embodiment 11-62. The engineered protease of any one of embodiments II-1 to 11-15, comprising a modified chymase protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 6.

Embodiment 11-63. The engineered protease of embodiment 11-62, wherein the modified chymase protease domain of SEQ ID NO: 6 comprises one of the mutation strings of Table 7B.

Embodiment 11-64. The engineered protease of any one of embodiments II-1 to 11-15, comprising a modified membrane type serine protease 1 (MTSP-1) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 7.

Embodiment 11-65. The engineered protease of embodiment 11-64, wherein the modified MTSP-1 protease domain of SEQ ID NO: 7 comprises one of the mutation strings of Table 5B.

Embodiment 11-66. The engineered protease of any one of embodiments II-1 to 11-15, comprising a modified urokinase-type plasminogen activator (uPA) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 22.

Embodiment 11-67. The engineered protease of embodiment 11-66, wherein the modified uPA protease domain of SEQ ID NO: 22 comprises one of the mutation strings of Table 3B.

Embodiment 11-68. The engineered protease of embodiment II-1, comprising a modified Kallikrein-related peptidase 5 (KLK5) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 23.

Embodiment 11-69. A method of inactivating Factor B, comprising contacting the Factor B with any of the engineered proteases of embodiments II-1 to 11-68.

Embodiment 11-70. A method of treating a disease or condition in a subject in need thereof, comprising administering to the subject any one of the engineered proteases of embodiments II-1 to 11-68.

Embodiment 11-71. The method of embodiment 11-70, wherein the disease or condition is associated with dysregulated complement.

Embodiment 11-72. The method of any one of embodiments 11-70 to 11-71, wherein the disease or condition is an inflammatory disease or condition.

Embodiment 11-73. The method of any one of embodiments 11-70 to 11-72, wherein the treatment is a replacement therapy.

Embodiment 11-74. The method of any one of embodiments 11-70 to 11-73, wherein the treatment blocks complement activation.

Embodiment 11-75. The method of any one of embodiments 11-70 to 11-74, wherein the treatment modulates autoimmunity.

Embodiment 11-76. The method of any one of embodiments 11-70 to 11-75, wherein the disease or condition is a congenital complement deficiency.

Embodiment 11-77. The method of any one of embodiments 11-70 to 11-76, wherein the treatment is for endothelial or kidney cell injury.

Embodiment 11-78. The method of any one of embodiments 11-70 to 11-77, wherein the disease or condition is selected from lupus nephritis, C3 glomerulopathy (C3G), primary IgA nephropathy, kidney transplant ischemia and reperfusion (I/R) injury, antineutrophil cytoplasmic antibody (ANCA)-associated vasculitis (AAV), sepsis, acute respiratory distress syndrome (ARDS), SARS-associated coronavirus (SARS-CoV), atypical hemolytic uremic syndrome (aHUS), membranous nephropathy (MN) and paroxysmal nocturnal hemoglobinuria (PNH).

Embodiment 11-79. The method of any one of embodiments 11-70 to 11-78, wherein the disease or condition is a control protein deficiency.

Embodiment 11-80. The method of any one of embodiments 11-70 to 11-78, wherein the disease or condition is a secondary complement disorder.

Embodiment 11-81. The method of any one of embodiments 11-70 to 11-78, wherein the disease or condition is an immunity-related disease or condition.

Embodiment 11-82. The method of any one of embodiments 11-70 to 11-81, wherein the engineered protease is administered to the subject subcutaneously.

Embodiment 11-83. The method of embodiment 11-82, wherein the engineered protease is activated in situ at the site of a dysregulated complement component.

Embodiment 11-84. The method of any of any one of embodiments 11-70 to 11-83, wherein the engineered protease is provided in a liquid stable formulation.

Embodiment 11-85. A pharmaceutical composition comprising any of the engineered proteases of embodiments II-1 to 11-68, and optionally a pharmaceutically acceptable carrier.

Embodiment 11-86. The pharmaceutical composition of embodiment 11-85, wherein the engineered protease is provided in a liquid stable formulation.

Embodiment 11-87. The pharmaceutical composition of any one of embodiments 11-85 to 11-86, wherein the composition is formulated for subcutaneous administration.

### Examples

### Example 1: Expression and Purification of Chymotrypsin-Like Serine Proteases and Initial Characterization

Expression and Purification of MTSP-1- and uPA-Based Proteases

Small-scale expression and purification of engineered MTSP-1- and uPA-based proteases was performed. Briefly, MTSP-1 and uPA each were expressed as a zymogen in an E. coli strain of BL21-Gold (DE3) bacterial host, isolated from inclusion bodies, denatured, refolded by rapid dilution, dialyzed and subsequently activated on an immobilized trypsin column. The active protein was then purified on an anion exchange column. Similarly, large-scale refold and purification of MTSP-1 was also performed. The MTSP-1 and uPA were solubilized, refolded, purified on an anion exchange column. Next, endotoxin was removed from the MTSP-1 and uPA.

### Expression and Purification of Chymase-Based Proteases

Small-scale expression and purification of chymase was performed. Briefly, chymase was expressed as a zymogen in inclusion bodies in an E. coli strain of BL21 Gold (DE3) bacterial host. The insoluble chymase was isolated from inclusion bodies, denatured in the presence of reducing agent, refolded by rapid dilution, and dialyzed. The zymogen form of the protein was purified using 4 mL of Fast Flow SP beads packed in BioRAD columns using gravity and activated overnight with enterokinase. The native chymase was then purified from the zymogen using cation exchange chromatography using a step elution method.

### Factor B Cleavage by Chymase-Based Engineered Proteases

Factor B cleavage by chymase-based engineered proteases was tested and verified by Coomassie gel. Briefly, a digestion reaction was prepared with 2.0 μM of Human Factor B (Complement Technologies) in 20 μL buffer (50 mM Tris pH 7.4/50 mM NaCl/0.01% Tween 20). Different concentration of the chymase-based engineered proteases were added to the Factor B (3000 nM at the top, diluted 1:2, 10 steps, including 0.0 nM) and were incubated for 1 hour at 37° C. After digestion, 15 μL of the reaction was transferred to a 96 well plate with 1.5 μL of 0.2N HCL to stop the chymase digestion. After quenching, the reactions were prepared for SDS-PAGE. 20 μL of the reaction mixtures were loaded per well of a 4-12% Bis-Tris Criterion gel. Densitometry analysis of the Factor B cleavage was performed, and EC₅₀ was calculated. FIG. 1D depicts a Coomassie gel showing examples of Factor B cleavage by the tested chymase-based engineered proteases. Table 8A lists the chymase-based engineered proteases tested in FIG. 1D. All references to engineered proteases in the table are depicted in chymotrypsin numbering. See Table 6 for the chymotrypsin numbering key for the modified protease domain of chymase. Two lots of each engineered protease set were used, and each lot showed the ability of the engineered proteases to cleave Factor B. Lot 1 of each set showed a visible amount of zymogen which is not titratable, but possibly is activated or is somewhat active on Factor B. These results also show that the engineered protease is highly efficient at Factor B cleavage. The engineered proteases tested are shown in Table 8A below.

FIG. 1E depicts a graph showing examples of Factor B cleavage by two chymase-based engineered proteases having low EC₅₀ calculated from ELISA measurements. All references in this Example to engineered proteases are depicted in chymotrypsin numbering. See Table 6 for the chymotrypsin numbering key for the modified protease domain of chymase. Briefly, a digestion reaction was prepared with 2.0 μM of Human Factor B (Complement Technologies) in 20 μL buffer (50 mM Tris pH 7.4/50 mM NaCl/0.01% Tween 20). Different concentrations of two chymase-based engineered proteases were used: C22S/P38Q/K40L/F41R/V138I/F173Y/D175N/A190SN213A/S218V/A226R and C22S/P38Q/K40M/F41R/V138I/F173Y/D175R/A190SN213A/S218V/A226R. These were each provided at 3000 nM, and diluted 1:2 to obtain 1500, 750, 375, 188, 94, 47, 23, 12, 6, and 3 nM, and additionally a control at 0.0 nM was used. These were each added to 2 μM of Factor B and incubated for 1 hour at 37° C. After digestion, 15 μL of the reaction was transferred to a 96 well plate with 1.5 μL of 0.2N HCL. After quenching, the reactions were prepared Factor B ELISA.

A Factor B standard curve was made with 800, 533.3, 355.5, 237.0, 158.0, 105.3, 70.2, 46.8, 31.2, 20.8, 0.0 μM in 1% BSA-PBST. A 384 well plate was coated with the monoclonal antibody against the human Factor Ba fragment (Quidel) at 2 μg/ml in carbonate buffer (25 μL/well). After blocking for 1 hour at room temperature with 1% BSA-PBST (100 μL/well), the digests (chymase+FB) were diluted to 800 pM and the standards were diluted 1:1.5 from 800 pM into the blocking buffer (25 μL/well). The plate was agitated for 30 minutes at room temperature.

The biotinylated monoclonal antibody against the human Factor Bb fragment (Quidel) was then added at 0.125 μg/ml to detect the bound FB. Streptavidin-HRP was then diluted at 1:200 in blocking buffer (25 μL/well) and the plate was agitated for 30 minutes at room temperature. The plate was developed with ELISABright, (50 μL/well) for 1 minute at room temperature and read in the EnVision plate reader. Table 8B below presents the EC₅₀ calculated for the two engineered proteases. FIG. 1E shows that 30-42 nM of the chymase-based engineered proteases tested were required to inhibit 50% of Factor B.

TABLE 8A
Chymase-Based Engineered Proteases Tested in FIG. 1D

| Notation in | |
|---|---|
| FIG. 1D | Mutation of Engineered Protease |
| 1 | C22S/K40L/F41K/L99N/F173Y/D175N/S218L/A226R |
| 2 | C22S/P38Q/K40M/F41R/L99M/F173Y/D175N/V213Q/S218V/A226R/ |
| | F228A |
| 3 | C22S/P38Q/K40M/F41R/D175N/V213Q/S218V/A226R/F228A |
| 4 | C22S/P38Q/K40L/F41R/L99H/V138I/F173Y/D175S/A190S/V213A/ |
| | S2181/A226R |

TABLE 8B
EC50 of Factor B Cleavage by Chymase-Based Engineered Proteases

| | C22S/P38Q/K40L/F41R/V138I/F173Y/ | C22S/P38Q/K40M/F41R/V138I/F173Y/ |
|---|---|---|
| | D175N/A190S/V213A/S218V/A226R | D175R/A190S/V213A/S218V/A226R |
| EC₅₀ | 42.47 | 30.22 |

### Example 2: Protein Characterization and Active Site Titration of Chymase-Based Engineered Proteases

Selected chymase-based engineered proteases were tested to measure the activity of these proteases, by using an active site titration method based on reaction with the inhibitory serpin bait, followed by HPLC analysis to quantitate the active fraction. All references in this Example, including Table 9, to engineered proteases are depicted in chymotrypsin numbering. See Table 6 for the chymotrypsin numbering key for the modified protease domain of chymase. The HPLC analysis detects a shift in the measured peak when the serpin bait is present, an indication that the protease is binding the bait and therefore active. Briefly, a working concentration of 24 μM of serpin bait was prepared from stock solution, with 50 μM of low molecular weight heparin for antithrombin (AT)-based baits. Protease solutions were prepared by using 30 μL of a 5 μM working solution. Reaction mixtures were incubated for 2 hours at 37° C., and each reaction mixture (protease alone or protease with bait) was analyzed by HPLC using standard protocols. All protease alone samples were run first, then followed by the protease with serpin bait samples. A summary of the results for the proteases tested is summarized in Table 9 below. k_{cat}/Kₘ was measured using EGVD/AE-QF (SEQ ID NO: 52) and second order rate constant K*ₐₚₚ was measured using EGVD/AE-ACT (SEQ ID NO: 53). These results measured the protease activity after purification, and most produced chymase-based engineered proteases tested showed an activity of between 60%420%.

TABLE 9
Active Site Titration of Chymase-Based Engineered Proteases Summary

| | | k*ₐₚₚ |
|---|---|---|
| | kcat/Km | EGVD/AE- |
| | EGVD/AE-QF | ACT |
| | (SEQ ID NO: | (SEQ ID |
| Mutation String | 52) | NO: 53) |
| C22S | | 30.2 |
| C22S/A226R | 347 | 77 |
| C22S/L99H/F173L/K192R/Y215H/R217T/S218V/A226R | 86 | 341 |
| C22S/L99H/F173I/K192R/Y215K/R217T/S218I/A226R | 202 | 1041 |
| C22S/L99H/F173L/K192R/Y215H/R217T/S218V/A226R | 374 | 2412 |
| C22S/F41I/L99W/G151L/F173K/R217S/A226R | 380 | 1732 |
| C22S/F41R/L99G/G151T/F173Y/Y215R/R217M/A226R | 385 | 773 |
| C22S/F41R/L99H/G151T/F173Y/Y215K/R217M/A226R | 197 | 1495 |
| C22S/F41R/L99Y/G151T/F173K/R217S/A226R | 68 | 684 |
| C22S/F41V/L99F/G151R/F173K/R217S/A226R | 102 | 652 |
| C22S/F41V/L99H/G151R/F173L/Y215R/R217T/A226R | 363 | 1131 |
| F41V/L99H/G151V/F173Y/Y215K/R217G/A226R | 457 | 649 |
| C22S/F41V/L99W/G151L/F173K/R217S/A226R | 413 | 1359 |
| C22S/K192R/A226R | 243 | 147 |
| C22S/L99R/F173L/K192R/Y215H/R217T/S218V/A226R | 229 | |
| C22S/L99Y/F173L/K192R/Y215H/R217T/S218V/A226R | 224 | |
| C22S/L99H/F173Y/K192R/S218L/A226R | 449 | |
| C22S/L99Y/F173Y/K192R/S218L/A226R | 231 | |
| C22S/K40L/F41A/K192R/A226R | 60 | |
| C22S/K40M/F41V/K192R/A226R | 32 | |
| C22S/K40M/F41V/G151R/K192R/A226R | 31 | |
| C22S/L99H/F173Y/K192R/Y215K/R217S/S218V/A226R | 262 | |
| C22S/L99F/F173K/K192R/R217S/S218I/A226R | 86 | |
| C22S/L99W/F173K/K192R/R217S/S218V/A226R | 128 | |
| C22S/L99W/F173K/R217S/S218R/A226R | 810 | |
| C22S/L99Q/F173L/Y215K/R217T/S218L/A226R | 55 | |
| C22S/F41R/L99H/G151S/F173Y/Y215K/R217M/A226R | 71 | |
| C22S/F41I/L99H/G151K/F173Y/Y215K/R217G/A226R | 89 | |
| C22S/F41V/L99H/G151R/F173S/Y215K/A226R | 53 | |
| C22S/F41R/L99H/F173Y/Y215K/R217M/A226R | 30 | |
| C22S/F41K/L99H/G151A/F173Y/Y215K/R217S/A226R | 355 | |
| C22S/F41R/L99H/G151A/F173S/Y215K/R217T/A226R | 52 | |
| C22S/F173Y/D175N/A226R | 2687 | |
| C22S/K40L/F41R/L99N/F173Y/S218L/A226R | 197 | |
| C22S/K40L/F41H/L99N/F173Y/S218L/A226R | 190 | |
| C22S/K40L/F41K/L99N/F173Y/S218L/A226R | 194 | |
| C22S/K40L/F41R/L99N/F173Y/D175N/S218L/A226R | 793 | |
| C22S/K40L/F41H/L99N/F173Y/D175N/S218L/A226R | 638 | |
| C22S/K40L/F41K/L99N/F173Y/D175N/S218L/A226R | 721 | |
| C22S/K40M/F41R/L99Q/F173Y/D175R/S218V/A226R | 157 | |
| C22S/K40M/F41R/L99Q/F173Y/D175R/S218I/A226R | 241 | |
| C22S/P38Q/V138I/A190S/V213A/A226R | 241 | |
| C22S/P38Q/V213Q/A226R/F228A | 1030 | |
| C22S/P38Q/K40A/F41R/L99H/D175N/V213Q/S218I/A226R/F228A | 180 | |
| C22S/P38Q/K40M/F41R/L99M/F173Y/D175N/V213Q/S218V/A226R/ | 181 | |
| F228A | | |
| C22S/P38Q/K40M/F41R/D175N/V213Q/S218V/A226R/F228A | 198 | |
| C22S/P38Q/K40L/F41R/V138I/F173Y/D175N/A190S/V213A/S218V/ | 1389 | |
| A226R | | |
| C22S/P38Q/K40L/F41R/L99H/V138I/F173Y/D175S/A190S/V213A/S | 832 | |
| 218I/A226R | | |
| C22S/P38Q/K40M/F41R/L99F/V138I/F173S/D175H/A190S/V213A/S | 95 | |
| 218V/A226R | | |
| C22S/P38Q/K40A/F41R/L99H/V138I/F173Y/D175N/A190S/V213A/ | 863 | |
| S218V/A226R | | |
| C22S/P38Q/K40A/F41K/L99H/V138I/D175S/A190S/V213A/S218V/ | 608 | |
| A226R | | |
| C22S/P38Q/K40M/F41R/L99N/V138I/F173Y/D175T/A190S/V213A/ | 398 | |
| S218V/A226R | | |
| C22S/P38Q/K40M/F41R/V138I/F173Y/D175R/A190S/V213A/S218V/ | 1797 | |
| A226R | | |
| C22S/P38Q/K40L/F41R/L99N/V138I/F173Y/D175N/A190S/V213A/ | 420 | |
| S218V/A226R | | |

### Example 3: Stability Studies of Serine Proteases Using Factor B Cleavage and Selection of Serine Proteases

Cleavage of Factor B by various engineered proteases was evaluated. Engineered chymase-, MTSP-1-, and uPA-based proteases were tested. All references in this Example, including Tables 10A, 10B, 11A, and 11B, to engineered proteases are depicted in chymotrypsin numbering. The chymotrypsin numbering key for the modified protease domains of engineered proteases are found in Tables 2, 4, and 6 for uPA, MTSP-1, and chymase, respectively. These experiments were also performed to evaluate the stability of non-naturally occurring chymases in various media, including cynomolgus monkey vitreous humor, human plasma, and phosphate buffered saline (PBS). Briefly, Factor B was diluted into an assay buffer and incubated at 37° C. with naturally occurring (wild type) and non-naturally occurring (engineered) proteases in vitreous humor, plasma, or PBS, and the reactions were quenched with HCl at various time points. The time points tested for chymase include T=0 which used 6 hours pre-incubation, T=2 hours which used 4 hours pre-incubation, T=4 hours which used 2 hours pre-incubation, and T=6 hours which used 0 hours pre-incubation. Assaying of the sample was performed with ELISA, or alpha screen for human C3 cleavage, using standard protocols.

A summary of the results is presented in Tables 10A-10B below, showing the various tested proteases, the concentration of the protease used in nM and the EC₅₀ of human Factor B cleavage. Table 10 presents human Factor B cleavage by various non-naturally occurring chymase-based engineered proteases, tested in 80% human plasma, compared against a chymase comprising the mutation string C122S. Blank cells indicate that a test was not performed for a particular protease mutation string. These results generally show that selected chymase-based engineered proteases such as C22S/P38Q/K40L/F41R/V138I/F173Y/D175N/A190SN213A/S218V/A226R and C22S/P38Q/K40M/F41R/V138I/F173Y/D175R/A190SN213A/S218V/A226R show high Factor B cleavage activity.

Table 10B presents human Factor B cleavage by various engineered chymase-, MTSP-1-, or uPA-based proteases, tested in mouse or human plasma. For ELISA analysis of Factor B cleavage, briefly, anti-Ba was used, and Bb was detected with anti-Bb. A dynamic range of 25-1600 pM was used. Half-life in 50% human plasma was also calculated for various chymase-based engineered proteases.

These results showed that the proteases engineered from the uPA-based scaffold did not show efficient Factor B cleavage, and proteases engineered from the MTSP-1-based scaffold showed Factor B cleavage but only for two candidates below 50 nM. The proteases engineered from the chymase-based scaffold showed the most efficient Factor B cleavage with EC₅₀ values below 50 nM.

TABLE 10A
Chymase-Based Engineered Proteases EC50 For Cleavage of Factor B

| | | | | | t_{1/2} in 50% |
|---|---|---|---|---|---|
| | | | fB | fB | human |
| | | fB | Cleavage, | Cleavage, | plasma |
| | fB | Cleavage, | 64% | 64% A₂M- | (min) |
| | Cleavage | 80% | plasma | depl | (EGVD′A |
| | EC₅₀ | plasma | EC₅₀ | plasma | E)(SEQ ID |
| Mutation String | (nM) | EC₅₀ (nM) | (nM) | EC₅₀ (nM) | NO: 13) |
| C22S | 265 | 10200 | 7885 | 1519 | |
| C22S/A226R | 5205 | none | | | |
| C22S/L99H/F173L/K192R/Y215H/ | 7770 | | | | |
| R217T/S218V/A226R | | | | | |
| C22S/L99H/F173I/K192R/Y215K/ | 5618 | | | | |
| R217T/S218I/A226R | | | | | |
| C22S/L99H/F173L/K192R/Y215H/ | 2738 | | | | |
| R217T/S218V/A226R | | | | | |
| C22S/F41I/L99W/G151L/F173K/ | 1741 | | | | |
| R217S/A226R | | | | | |
| C22S/F41R/L99G/G151T/F173Y/ | 4329 | | | | |
| Y215R/R217M/A226R | | | | | |
| C22S/F41R/L99H/G151T/F173Y/ | 1690 | | | | |
| Y215K/R217M/A226R | | | | | |
| C22S/F41R/L99Y/G151T/F173K/ | 3447 | | | | |
| R217S/A226R | | | | | |
| C22S/F41V/L99F/G151R/F173K/ | 3646 | | | | |
| R217S/A226R | | | | | |
| C22S/F41V/L99H/G151R/F173L/ | 8060 | | | | |
| Y215R/R217T/A226R | | | | | |
| F41V/L99H/G151V/F173Y/Y215K/ | 7770 | | | | |
| R217G/A226R | | | | | |
| C22S/F41V/L99W/G151L/F173K/ | 1944 | | | | |
| R217S/A226R | | | | | |
| C22S/K192R/A226R | 6367 | | | | 17.3 |
| C22S/L99R/F173L/K192R/Y215H/ | 7770 | 7590* | 4380 | 2290 | 20.9 |
| R217T/S218V/A226R | | | | | |
| C22S/L99Y/F173L/K192R/Y215H/ | 4880-6304 | | 12406 | 4575 | 10.1 |
| R217T/S218V/A226R | | | | | |
| C22S/L99H/F173Y/K192R/S218L/ | 1147 | | | | |
| A226R | | | | | |
| C22S/L99R/F173Y/K192R/S218L/ | 555 | | | | |
| A226R | | | | | |
| C22S/L99Y/F173Y/K192R/S218L/ | 1020 | | | | |
| A226R | | | | | |
| C22S/K40L/F41A/K192R/A226R | 7390 | | | | |
| C22S/K40L/F41G/K192R/A226R | 1020- | | | | |
| | 8540 | | | | |
| C22S/K40M/F41V/K192R/A226R | 7770 | | | | |
| C22S/K40M/F41V/G151R/K192R/ | 7770 | | | | |
| A226R | | | | | |
| C22S/K40L/F41V/G151R/K192R/ | 7500 | | | | |
| A226R | | | | | |
| C22S/L99H/F173Y/K192R/Y215K/ | 3456 | | | | |
| R217S/S218V/A226R | | | | | |
| C22S/L99F/F173K/K192R/R217S/ | 4001 | | | | |
| S218I/A226R | | | | | |
| C22S/L99W/F173K/K192R/R217S/ | 2492 | | | | |
| S218V/A226R | | | | | |
| C22S/L99W/F173K/R217S/S218R/ | 2663 | | | | |
| A226R | | | | | |
| C22S/L99Q/F173L/Y215K/R217T/ | (weak) | | | | |
| S218L/A226R | | | | | |
| C22S/F41R/L99H/G151S/F173Y/ | 3387- | | | | |
| Y215K/R217M/A226R | 2730 | | | | |
| C22S/F41I/L99H/G151K/F173Y/ | 7770 | | | | |
| Y215K/R217G/A226R | | | | | |
| C22S/F41V/L99H/G151R/F173S/ | 4781 | | | | |
| Y215K/A226R | | | | | |
| C22S/F41R/L99H/F173Y/Y215K/ | 6518- | | | | |
| R217M/A226R | 5390 | | | | |
| C22S/F41K/L99H/G151A/F173Y/ | 1313 | 18600 | 5692 | 5752 | |
| Y215K/R217S/A226R | | | | | |
| C22S/F41R/L99H/G151A/F173S/ | 2250 | | 3402 | 2879 | |
| Y215K/R217T/A226R | | | | | |
| C22S/F41L/L99H/G151T/F173Y/ | 3230 | | | | |
| Y215K/A226R | | | | | |
| C22S/F41R/L99H/G151T/F173Y/ | 3220 | | | | |
| Y215K/A226R | | | | | |
| C22S/F41G/L99H/G151T/F173Y/ | 6600 | | | | |
| Y215K/A226R | | | | | |
| C22S/F41R/L99H/F173Y/Y215K/ | 4800 | | | | |
| A226R | | | | | |
| C22S/F41V/L99H/F173Y/Y215K/ | 6060 | | | | |
| A226R | | | | | |
| C22S/F41G/L99H/F173Y/Y215K/ | 6650 | | | | |
| A226R | | | | | |
| C22S/F41R/L99H/G151T/F173L/ | 4800 | | | | |
| Y215K/A226R | | | | | |
| C22S/F41V/L99H/G151T/F173L/ | 5420 | | | | |
| Y215K/A226R | | | | | |
| C22S/F41G/L99H/G151T/F173L/ | 11500 | | | | |
| Y215K/A226R | | | | | |
| C22S/F41R/L99H/F173L/Y215K/ | 6640 | | | | |
| A226R | | | | | |
| C22S/F41V/L99H/G151T/F173Y/ | 5330 | | | | |
| Y215K/A226R | | | | | |
| C22S/F41L/L99H/F173Y/Y215K/ | 3450 | | | | |
| A226R | | | | | |
| C22S/F41L/L99H/G151T/F173L/ | 4170 | | | | |
| Y215K/A226R | | | | | |
| C22S/F41L/L99H/F173L/Y215K/ | 7890 | | | | |
| A226R | | | | | |
| C22S/F41V/L99H/F173L/Y215K/ | 8730 | | | | |
| A226R | | | | | |
| C22S/F41G/L99H/F173L/Y215K/ | 10200 | | | | |
| A226R | | | | | |
| C22S/L99R/K192R/S218L/A226R | 7510 | | | | |
| C22S/L99R/F173Y/S218L/A226R | 546 | | | | 11.1 |
| C22S/L99R/F173Y/K192R/A226R | 3278 | | | | |
| C22S/K93Q/G119D/F173S/K192R/ | 7770 | | | | |
| A226R | | | | | |
| C22S/P38Q/H100P/L116R/F123L/ | 868-1120 | | | | 29 |
| Q180H/S218I/A226R/R235S | | | | | |
| E21G/C22S/R61H/M135T/F173Y/ | 1275- | | | | 23 |
| D175N/A209V/A226R | 1050 | | | | |
| C22S/K111E/F173L/Y215H/A226R | 9008 | | | | |
| C22S/F173L/Y215H/A226R | 5991 | | | | |
| C22S/M135T/P150S/S152G/Q166H/ | 3163 | | | | |
| D175N/A190T/A226R | | | | | |
| E21G/C22S/R61H/M135T/F173Y/ | 1260 | | | | |
| D175N/A209V/A226R | | | | | |
| C22S/P38Q/H100P/L116R/F123L/ | 3737 | | | | |
| Q180H/A226R/R235S | | | | | |
| C22S/V138I/L160V/S189T/A226R | 7132 | | | | |
| C22S/V138L/A226R | 3483 | | | | |
| C22S/V138L/A226R | 3483 | | | | |
| C22S/L160V/S189T/A226R | 6636 | | | | |
| C22S/S189T/A226R | 7984 | | | | |
| C22S/S218I/A226R | 830 | | | | 7.8 |
| C22S/F173Y/A226R | 650 | 21000 | | | 5.9 |
| C22S/D175N/A226R | 564 | 7770 | | | 6.2 |
| C22S/F173Y/D175N/A226R | 151-182- | 11200 | 5999- | 1647 | 7.3, 9.0 |
| | 177 | | 5616 | | |
| C22S/A220S/A226R | 5563 | | | | |
| C22S/S189T/A190S/F191S/A220L/ | 8141 | | | | |
| A226R | | | | | |
| C22S/K40L/F41R/L99N/F173Y/ | 379 | 15568 | | | 22.7 |
| S218L/A226R | | | | | |
| C22S/K40M/F41R/L99H/G151P/ | 1503- | | | | |
| F173S/S218V/A226R | 1910 | | | | |
| C22S/K40M/F41R/L99H/G151A/ | 3221- | | | | |
| F173S/S218V/A226R | 4710 | | | | |
| C22S/K40M/F41I/L99H/G151R/ | 8202 | | | | |
| F173S/S218L/A226R | | | | | |
| C22S/K40M/F41R/L99N/G151S/ | 320-445 | 11193 | | | 10.9 |
| F173Y/S218L/A226R | | | | | |
| C22S/K40M/F41R/L99N/G151P/ | 2575- | | | | |
| F173S/S218V/A226R | 3010 | | | | |
| C22S/K40M/F41R/L99H/G151P/ | 1864- | | | | |
| F173S/S218I/A226R | 2100 | | | | |
| C22S/K40L/F41H/L99N/F173Y/ | 284-530 | 13903 | | | 28.9 |
| S218L/A226R | | | | | |
| C22S/K40M/F41V/L99N/G151R/ | | | | | |
| F173S/S218V/A226R | | | | | |
| C22S/K40L/F41L/L99N/G151H/ | 304-746 | 17076 | | | 20.4 |
| F173Y/S218V/A226R | | | | | |
| C22S/K40L/F41K/L99H/G151S/ | 276-369 | 17234 | | | 20.1 |
| F173Y/S218L/A226R | | | | | |
| C22S/K40L/F41K/L99H/F173Y/ | 228-394 | 14813 | | | 13.9 |
| S218L/A226R | | | | | |
| C22S/K40L/F41K/L99N/F173Y/ | 405-373 | 13979 | | | 28.9 |
| S218L/A226R | | | | | |
| C22S/K40M/F41V/L99N/G151R/ | 11824 | | | | |
| F173S/S218E/A226R | | | | | |
| C22S/A190S/A226R | 407 | | | | 4.2 |
| C22S/S189T/A190S/A226R | 605 | none | | | |
| C22S/K40L/F41R/L99N/F173Y/ | 119 | 4092; 3756 | | | 23.7 |
| D175N/S218L/A226R | | | | | |
| C22S/K40L/F41H/L99N/F173Y/ | 139 | 5066 | | | 24.9 |
| D175N/S218L/A226R | | | | | |
| C22S/K40L/F41K/L99N/F173Y/ | 61-129 | 5697 | | | 27 |
| D175N/S218L/A226R | | | | | |
| C22S/P38Q/V138I/V183I/L199V/ | none | | | | |
| A226R/F228V | | | | | |
| C22S/V138T/A190S/V213A/A226R | 6708 | | | | |
| C22S/P38Q/V138L/A190G/L199V/ | none | | | | |
| V213A/A226R/F228A | | | | | |
| C22S/P38Q/V138I/A226R/F228A | 12395 | | | | |
| C22S/F41K/L99N/F173Y/D175N/ | 77 | | | | 30 |
| S218L/A226R | | | | | |
| C22S/K40L/L99N/F173Y/D175N/ | 113 | | | | 11.4 |
| S218L/A226R | | | | | |
| C22S/K40L/F41K/F173Y/D175N/ | 77 | | | | 10.3 |
| S218L/A226R | | | | | |
| C22S/K40L/F41K/L99N/D175N/ | 295 | | | | 13.8 |
| S218L/A226R | | | | | |
| C22S/K40L/F41K/L99N/F173Y/ | 151 | | | | 15.1 |
| D175N/A226R | | | | | |
| C22S/V138A/G145D/S189T/A190S/ | 3813 | | | | |
| A226R | | | | | |
| C22S/A226R/F228A | 5072 | | | | |
| C22S/V138A/S189T/A226R/F228M | 1772 | | | | |
| C22S/V138L/S189T/A190G/A226R | 4160 | | | | |
| C22S/V138S/S189T/A226R/F228M | 2985 | | | | |
| C22S/S189T/A226R/F228M | 1553 | | | | |
| C22S/V138A/A226R | 2942 | | | | |
| C22S/V138A/S189T/A226R | 4420 | | | | |
| C22S/K40M/F41R/L99N/F173Y/ | 230 | 5137 | | | 29.6 |
| D175S/S218L/A226R | | | | | |
| C22S/K40M/F41R/L99H/F173Y/ | 181 | 7213 | | | 11.2 |
| D175W/S218L/A226R | | | | | |
| C22S/K40M/F41K/F173Y/D175R/ | 203 | 4130 | | | 38.8 |
| S218E/A226R | | | | | |
| C22S/K40M/F41K/L99N/F173Y/ | 216 | 4128 | | | 16.9 |
| D175R/S218T/A226R | | | | | |
| C22S/K40L/F41K/L99Q/F173Y/ | 110 | 5672 | | | 15.6 |
| D175N/S218V/A226R | | | | | |
| C22S/K40M/F41R/L99Q/F173Y/ | 235 | 4069 | | | 73.7 |
| D175R/S218V/A226R | | | | | |
| C22S/K40L/F41K/L99H/F173Y/ | 101 | 4621 | | | 11.5 |
| D175S/S218I/A226R | | | | | |
| C22S/K40L/F41R/L99G/F173Y/ | 333 | 8625 | | | 24.9 |
| D175R/S218V/A226R | | | | | |
| C22S/K40L/F41R/L99H/F173Y/ | 84 | 5561 | | | 10.4 |
| D175Y/S218I/A226R | | | | | |
| C22S/K40M/F41R/L99Q/F173Y/ | 213 | 4481 | | | 65.3 |
| D175R/S218I/A226R | | | | | |
| C22S/P38Q/V138I/A190S/V213A/ | 1348.4 | | | | |
| A226R | | | | | |
| C22S/P38Q/V213Q/A226R/F228A | 6210.6 | | | | |
| C22S/P38Q/K40A/F41R/L99H/ | 768.8 | | | | |
| F173Y/D175Y/V213Q/S218V/A226R/ | | | | | |
| F228A | | | | | |
| C22S/P38Q/K40A/F41R/L99H/ | 552.9 | | | | |
| D175N/V213Q/S218I/A226R/F228A | | | | | |
| C22S/P38Q/K40M/F41R/L99M/ | 787.5 | | | | |
| F173Y/D175N/V213Q/S218V/A226R/ | | | | | |
| F228A | | | | | |
| C22S/P38Q/K40M/F41R/D175N/ | 931.5 | | | | |
| V213Q/S218V/A226R/F228A | | | | | |
| C22S/P38Q/K40L/F41R/V138I/ | 36.3 | | | | |
| F173Y/D175N/A190S/V213A/S218V/ | | | | | |
| A226R | | | | | |
| C22S/P38Q/K40L/F41R/L99H/ | 60.3 | | | | |
| V138I/F173Y/D175S/A190S/V213A/ | | | | | |
| S218I/A226R | | | | | |
| C22S/P38Q/K40M/F41R/L99F/ | 625- | | | | |
| V138I/F173S/D175H/A190S/V213A/ | 796.6 | | | | |
| S218V/A226R | | | | | |
| C22S/P38Q/K40A/F41R/L99H/ | 71.8 | | | | |
| V138I/F173Y/D175N/A190S/V213A/ | | | | | |
| S218V/A226R | | | | | |
| C22S/P38Q/K40A/F41K/L99H/ | 142.3 | | | | |
| V138I/D175S/A190S/V213A/S218V/ | | | | | |
| A226R | | | | | |
| C22S/P38Q/K40M/F41R/L99N/ | 92.1 | | | | |
| V138I/F173Y/D175T/A190S/V213A/ | | | | | |
| S218V/A226R | | | | | |
| C22S/P38Q/K40M/F41R/V138I/ | 28.1 | | | | |
| F173Y/D175R/A190S/V213A/S218V/ | | | | | |
| A226R | | | | | |
| C22S/P38Q/K40L/F41R/L99N/ | 65.3 | | | | |
| V138I/F173Y/D175N/A190S/V213A/ | | | | | |
| S218V/A226R | | | | | |
| C22S/P38Q/K40A/F41R/V138I/ | 261.7 | | | | |
| D175R/A190S/V213A/S218T/A226R | | | | | |
| C22S/P38Q/K40A/F41R/L99H/ | 405.5 | | | | |
| V138I/F173Y/A190S/V213A/S218V/ | | | | | |
| A226R | | | | | |
| C22S/P38Q/K40M/F41H/L99N/ | 561 | | | | |
| V138I/F173Y/D175S/A190S/V213A/ | | | | | |
| S218T/A226R | | | | | |
| C22S/P38Q/K40M/F41K/L99Y/ | 448.1 | | | | |
| V138I/F173M/D175Y/A190S/V213A/ | | | | | |
| S218V/A226R | | | | | |
| C22S/P38Q/K40A/F41R/V138I/ | 1720.7 | | | | |
| F173S/D175T/A190S/V213A/S218V/ | | | | | |
| A226R | | | | | |
| C22S/P38Q/K40L/F41R/V138I/ | 314.2 | | | | |
| F173L/D175H/A190S/V213A/S218V/ | | | | | |
| A226R | | | | | |
| C22S/P38Q/K40M/F41R/L99N/ | 448.9 | | | | |
| V138I/D175N/A190S/V213A/S218T/ | | | | | |
| A226R | | | | | |
| C22S/P38Q/K40M/F41H/V138I/ | 47.6 | | | | |
| F173Y/D175R/A190S/V213A/ | | | | | |
| S218V/A226R | | | | | |
| C22S/P38Q/K40M/F41R/L99Q/ | 723.6 | | | | |
| V138I/F173Y/D175A/A190S/V213A/ | | | | | |
| S218T/A226R | | | | | |
| C22S/P38Q/K40A/F41R/L99H/ | 190.7 | | | | |
| V138I/D175R/A190S/V213A/S218L/ | | | | | |
| A226R | | | | | |
| C22S/P38Q/K40M/F41R/L99F/ | 870.1 | | | | |
| V138I/F173L/D175N/A190S/V213A/ | | | | | |
| A226R | | | | | |
| C22S/P38Q/K40M/F41R/L99Y/ | 814.2 | | | | |
| V138I/F173L/D175S/A190S/V213A/ | | | | | |
| S218V/A226R | | | | | |
| C22S/P38Q/K40L/F41R/V138I/ | 114.5 | | | | |
| D175N/A190S/V213A/S218V/A226R | | | | | |
| C22S/P38Q/K40M/F41R/L99Y/ | 102.7 | | | | |
| V138I/F173Y/D175S/A190S/V213A/ | | | | | |
| S218V/A226R | | | | | |
| C22S/P38Q/K40L/F41R/V138I/ | 456.4 | | | | |
| F173M/D175N/A190S/V213A/S218L/ | | | | | |
| A226R | | | | | |
| C22S/P38Q/K40L/F41R/L99N/ | 57.6 | | | | |
| V138I/F173Y/D175T/A190S/V213A/ | | | | | |
| S218V/A226R | | | | | |
| C22S/P38Q/K40M/F41R/V138I/ | 481.7 | | | | |
| F173L/D175N/A190S/V213A/S218L/ | | | | | |
| A226R | | | | | |
| C22S/P38Q/K40L/F41K/L99N/ | 143.5 | | | | |
| V138I/F173Y/D175S/A190S/V213A/ | | | | | |
| S218I/A226R | | | | | |
| C22S/P38Q/K40A/F41R/L99Y/ | 510.7 | | | | |
| V138I/F173L/D175R/A190S/V213A/ | | | | | |
| S218L/A226R | | | | | |
| C22S/P38Q/K40L/F41R/L99Q/ | 102.5 | | | | |
| V138I/F173Y/D175N/A190S/V213A/ | | | | | |
| S218V/A226R | | | | | |
| C22S/P38Q/K40M/F41R/L99Q/ | 157.3 | | | | |
| V138I/F173Y/D175N/A190S/V213A/ | | | | | |
| S218V/A226R | | | | | |
| C22S/P38Q/K40V/F41R/V138I/ | 169 | | | | |
| F173L/D175R/A190S/V213A/S218I/ | | | | | |
| A226R | | | | | |
| C22S/P38Q/K40M/F41T/V138I/ | 218.4 | | | | |
| D175R/A190S/V213A/S218I/A226R | | | | | |
| C22S/P38Q/K40M/F41R/V138I/ | 349.5 | | | | |
| F173L/D175K/A190S/V213A/S218V/ | | | | | |
| A226R | | | | | |
| C22S/P38Q/K40M/F41R/L99Y/ | 10564.6 | | | | |
| V138I/F173S/D175S/A190S/V213A/ | | | | | |
| S218T/A226R | | | | | |
| C22S/P38Q/K40A/F41T/L99Y/ | 3125.4 | | | | |
| V138I/F173S/D175H/A190S/V213A/ | | | | | |
| S218I/A226R | | | | | |
| C22S/P38Q/K40A/F41R/V138I/ | 282.9 | | | | |
| F173L/D175R/A190S/V213A/S218L/ | | | | | |
| A226R | | | | | |
| C22S/P38Q/K40M/F41R/L99Y/ | 1650.6 | | | | |
| V138I/F173L/D175Q/A190S/V213A/ | | | | | |
| S218T/A226R | | | | | |
| C22S/P38Q/K40A/F41Q/V138I/ | 2187.2 | | | | |
| F173S/D175W/A190S/V213A/S218V/ | | | | | |
| A226R | | | | | |
| C22S/P38Q/K40M/F41H/V138I/ | 1060.4 | | | | |
| D175S/A190S/V213A/A226R | | | | | |
| C22S/P38Q/K40V/F41R/V138I/ | 279.1 | | | | |
| F173L/D175N/A190S/V213A/S218V/ | | | | | |
| A226R | | | | | |
| C22S/P38Q/K40V/F41R/L99H/ | 638.9 | | | | |
| V138I/F173M/D175N/A190S/V213A/ | | | | | |
| S218T/A226R | | | | | |
| C22S/P38Q/K40L/F41R/V138I/ | 112.9 | | | | |
| F173L/D175R/A190S/V213A/S218V/ | | | | | |
| A226R | | | | | |
| C22S/P38Q/K40L/F41R/L99M/ | 212.8 | | | | |
| V138I/F173L/D175R/A190S/V213A/ | | | | | |
| S218V/A226R | | | | | |
| C22S/P38Q/K40A/F41R/L99H/ | 882 | | | | |
| V138I/F173M/D175T/A190S/V213A/ | | | | | |
| S218I/A226R | | | | | |
| C22S/P38Q/K40M/F41R/L99Y/ | 1225.5 | | | | |
| V138I/F173L/D175S/A190S/V213A/ | | | | | |
| S218T/A226R | | | | | |
| C22S/P38Q/K40A/F41R/L99H/ | 92.9 | | | | |
| V138I/F173Y/D175N/A190S/V213A/ | | | | | |
| S218I/A226R | | | | | |
| C22S/P38Q/K40M/F41R/L99W/ | 414.5 | | | | |
| V138I/F173M/D175H/A190S/V213A/ | | | | | |
| S218T/A226R | | | | | |
| C22S/P38Q/K40M/F41H/L99H/ | 277.4 | | | | |
| V138I/D175S/A190S/V213A/S218V/ | | | | | |
| A226R | | | | | |
| C22S/P38Q/K40V/F41R/V138I/ | 716.7 | | | | |
| F173S/D175N/A190S/V213A/S218I/ | | | | | |
| A226R | | | | | |
| C22S/P38Q/K40L/F41R/L99F/ | 631.2 | | | | |
| V138I/F173L/D175N/A190S/V213A/ | | | | | |
| A226R | | | | | |
| C22S/P38Q/K40M/F41R/L99H/ | 300.8 | | | | |
| V138I/D175Q/A190S/V213A/S218V/ | | | | | |
| A226R | | | | | |
| C22S/P38Q/K40L/F41R/V138I/ | 121.2 | | | | |
| F173Y/D175H/A190S/V213A/S218T/ | | | | | |
| A226R | | | | | |
| C22S/P38Q/K40M/F41K/L99W/ | 956.5 | | | | |
| V138I/F173M/A190S/V213A/S218N/ | | | | | |
| A226R | | | | | |
| C22S/P38Q/K40Q/F41R/V138I/ | 100.2 | | | | |
| F173Y/D175N/A190S/V213A/S218V/ | | | | | |
| A226R | | | | | |
| C22S/P38Q/K40I/F41R/L99N/V138I/ | 202.9 | | | | |
| D175T/A190S/V213A/S218V/ | | | | | |
| A226R | | | | | |
| C22S/P38Q/K40Q/F41R/L99H/ | 149.9 | | | | |
| V138I/F173Y/D175S/A190S/V213A/ | | | | | |
| S218V/A226R | | | | | |
| C22S/P38Q/K40A/F41R/L99H/ | 138.6 | | | | |
| V138I/F173Y/D175N/A190S/V213A/ | | | | | |
| S218L/A226R | | | | | |
| C22S/P38Q/K40M/F41R/L99N/ | 193 | | | | |
| V138I/F173Y/D175F/A190S/V213A/ | | | | | |
| S218V/A226R | | | | | |
| C22S/P38Q/K40M/F41R/L99N/ | 500 | | | | |
| V138I/F173Y/D175W/A190S/V213A/ | | | | | |
| S218T/A226R | | | | | |
| C22S/P38Q/K40L/F41R/L99Y/ | 57 | | | | |
| V138I/F173Y/D175N/A190S/V213A/ | | | | | |
| S218V/A226R | | | | | |
| C22S/P38Q/K40M/F41R/L99S/ | 472 | | | | |
| V138I/F173Y/D175N/A190S/V213A/ | | | | | |
| S218V/A226R | | | | | |
| C22S/P38Q/K40A/F41R/L99H/ | 170 | | | | |
| V138I/D175N/A190S/V213A/S218I/ | | | | | |
| A226R | | | | | |
| C22S/P38Q/K40M/F41R/V138I/ | 287 | | | | |
| F173S/D175Y/A190S/V213A/S218V/ | | | | | |
| A226R | | | | | |
| C22S/P38Q/K40M/F41R/V138I/ | 51 | | | | |
| F173L/D175R/A190S/V213A/S218T/ | | | | | |
| A226R | | | | | |
| C22S/P38Q/K40L/F41R/V138I/ | 223 | | | | |
| F173Y/D175R/A190S/V213A/S218T/ | | | | | |
| A226R | | | | | |
| C22S/P38Q/K40A/F41K/L99N/ | 121 | | | | |
| V138I/D175H/A190S/V213A/S218V/ | | | | | |
| A226R | | | | | |
| C22S/P38Q/K40M/F41R/V138I/ | 465 | | | | |
| F173Y/D175Y/A190S/V213A/S218I/ | | | | | |
| A226R | | | | | |

TABLE 10B
MTSP-1-, uPA-, and Chymase-Based Engineered
Proteases Cleavage Assays for EC₅₀ Factor B

| | | hu Factor B cleavage (ELISA) | | |
|---|---|---|---|---|
| | | EC₅₀ (nM) | | |
| Scaffold | Mutation String | n | Average | ±S.D. |
| uPA | V38D/A96E/D97G/C122S/Y172L/A98G/ | 1 | no cleavage | |
| | H99M/T97a_L97bdel | | @ 1.2 uM | |
| uPA | V38D/C122S/T97a_L97bdel | 1 | no cleavage | |
| | | | @ 3 uM | |
| uPA | V38D/A96G/D97E/A98G/H99V/C122S/ | 1 | no cleavage | |
| | T97a_L97bdel | | @ 0.95 uM | |
| uPA | C122S/V38D/A96N/D97A/A98G/H99V/ | 1 | no cleavage | |
| | T97a_L97bdel | | @ 1 uM | |
| uPA | V38D/C122S/D97L/A98G/H99V/ | 1 | no cleavage | |
| | T97a_L97bdel | | @ 0.95 uM | |
| uPA | V38D/C122S/A96E/D97R/A98G/H99V/ | 1 | no cleavage | |
| | T97a_L97bdel | | @ 3 uM | |
| uPA | V38D/C122S/A96E/D97H/A98G/H99L/ | 1 | no cleavage | |
| | T97a_L97bdel | | @ 1.05 uM | |
| uPA | V38D/A96E/D97E/C122S/A98G/H99A/ | 1 | no cleavage | |
| | T97a_L97bdel | | @ 0.75 uM | |
| uPA | V38D/C122S/A96D/D97G/A98G/H99A/ | 1 | no cleavage | |
| | T97a_L97bdel | | @ 1.1 uM | |
| uPA | V38D/D97A/C122S/A98G/H99L/ | 2 | 6560.0 | 8145.9 |
| | T97a_L97bdel | | | |
| uPA | V38D/C122S/A96D/D97W/A98N/H99L/ | 1 | 3720.0 | |
| | T97a_L97bdel | | | |
| uPA | V38D/C122S/A96D/D97T/A98G/H99M/ | 1 | 7860.0 | |
| | T97a_L97bdel | | | |
| uPA | V38D/A98G/H99A/C122S/T97a_L97bdel | 1 | no cleavage | |
| | | | @ 0.8 uM | |
| uPA | V38D/A98G/H99A/C122S/L97bdel | 1 | no cleavage | |
| | | | @ 1.6 uM | |
| uPA | V38D/A98G/H99L/C122S/T97a_L97bdel | 1 | no cleavage | |
| | | | @ 0.55 uM | |

Further, various chymase-based engineered proteases were tested to evaluate their baseline stability, by testing the effects of indirect and MTSP buffer (+/−EDTA) on the chymase-based engineered proteases. The peptide substrate EGVDAE-QF (SEQ ID NO: 52) was used for these experiments.

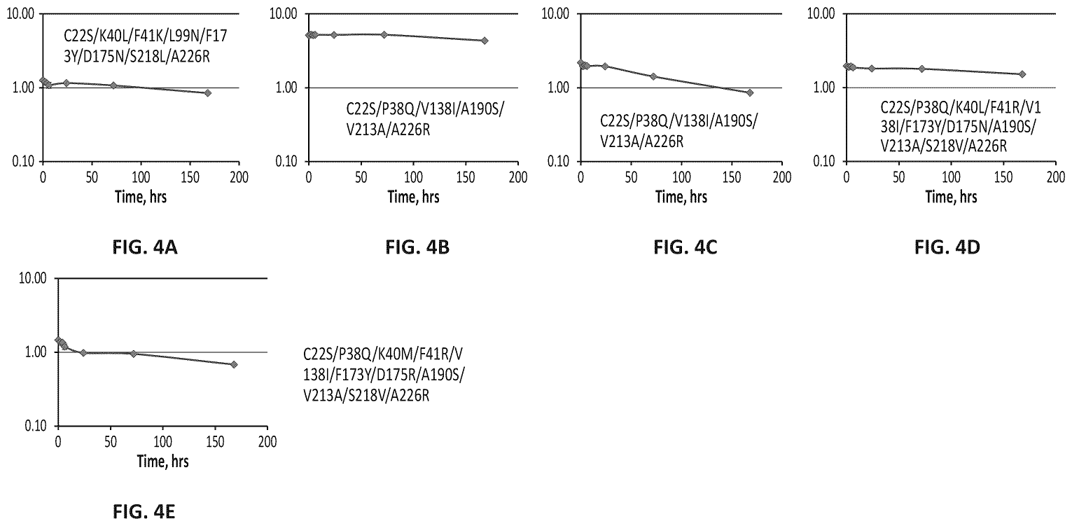

FIGS. 4A-4E depict graphs showing the stability of five different chymase proteases tested using a peptide substrate, in PBS, at 37° C. Table 11A summarizes the data shown in FIGS. 4A-4E, and lists the engineered proteases tested.

TABLE 11A
Activity of Chymase-based Engineered Proteases Tested with Peptide Substrate

| | | Fractional remaining activity | | | | | | |
|---|---|---|---|---|---|---|---|---|
| | Mutation of | | | | | | 72 hrs | 168 hrs |
| Figure | Engineered Protease | 0 hr | 2 hrs | 4 hrs | 6 hrs | 24 hrs | (Day 3) | (Day 7) |
| FIG. | C22S/K40L/F41K/L99N/ | 1.00 | 0.96 | 0.91 | 0.86 | 0.92 | 0.85 | 0.67 |
| 4A | F173Y/D175N/S218L/A226R | | | | | | | |
| FIG. | C22S/P38Q/V138I/A190S/ | 1.00 | 0.96 | 0.91 | 0.86 | 0.92 | 0.85 | 0.67 |
| 4B | V213A/A226R | | | | | | | |
| FIG. | C22S/P38Q/V138I/A190S/ | 1.00 | 1.02 | 1.01 | 1.02 | 1.01 | 1.02 | 0.85 |
| 4C | V213A/A226R | | | | | | | |
| FIG. | C22S/P38Q/K40L/F41R/ | 1.00 | 0.97 | 0.99 | 0.95 | 0.92 | 0.91 | 0.77 |
| 4D | V138I/F173Y/D175N/A190S/ | | | | | | | |
| | V213A/S218V/A226R | | | | | | | |
| FIG. | C22S/P38Q/K40M/F41R/ | 1.00 | 0.94 | 0.92 | 0.82 | 0.67 | 0.60 | 0.47 |
| 4E | V138I/F173Y/D175R/A190S/ | | | | | | | |
| | V213A/S218V/A226R | | | | | | | |

As shown, these proteases were more stable in the indirect buffer having a pH of 7.4. Wild type or naturally occurring chymase showed a loss of activity in all buffers, and EDTA had no apparent effect on stability.

Next, the second order rate constant (k*) was measured for the inhibition of various non-naturally occurring chymase-based proteases by plasma, for the extrapolation of the half-life in 100% plasma. Briefly, a baseline chymase having the modifications C22S/A226R was used. The concentration of the baseline chymase and the tested chymase-based engineered proteases, listed in Table 11B below, were used at 50 nM, and another tested chymase C22S/L99R/F173Y/K192R/S218L/A226R was used at 25 nM. For the baseline chymase, a time course of protease activity in the presence of plasma was measured using 4.2 uM CPQ2-ITLLSA-K(5FAM)-K-PEG8-K(biotin)-NH2 (SEQ ID NO: 17)/21 uM Neutravidin at 37° C. A summary of the results is presented in Table 11B below.

TABLE 11B
Second Order Rate Constant Measurements and Calculated t_{1/2}

| | | calculated t_{1/2} (s) |
|---|---|---|
| Mutation string | k* (%⁻¹s⁻¹) | (in 100% plasma) |
| C22S/A226R | 7.32E−5 | 95 |
| C22S/L99R/F173Y/ | 6.24E−5 | 111 |
| K192R/S218L/A226R | | |
| C22S/L99Y/F173Y/ | 3.71E−5 | 187 |
| K192R/S218L/A226R | | |

### Example 4: Factor B Add-Back Hemolysis Assay

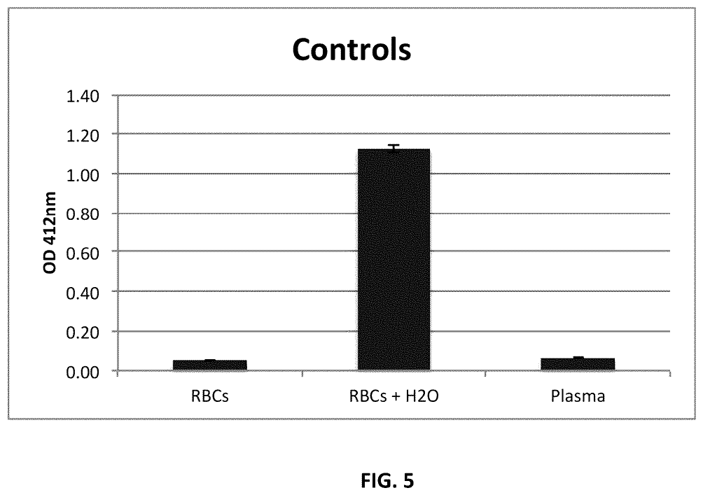

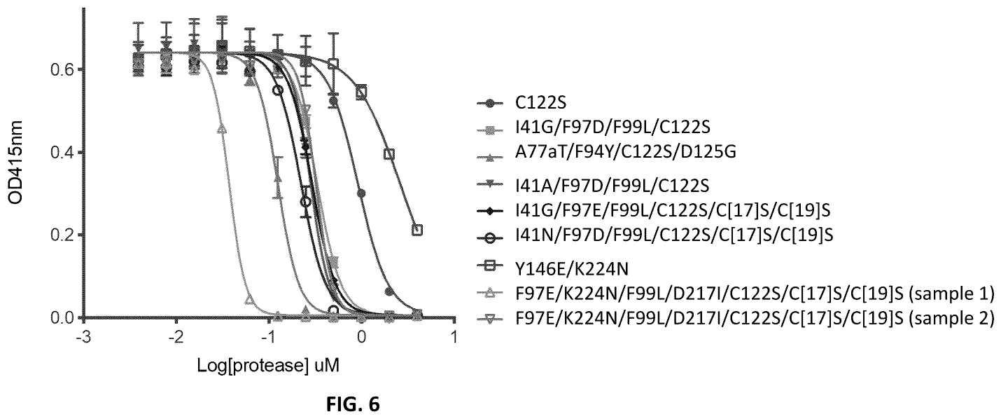

Hemolysis assays were performed to evaluate the hemolytic activity of various lots of Factor B, a lot obtained from CompTech as compared to a lot expressed in-house. Briefly, Factor B depleted human serum was used, and plasma purified or recombinant human Factor B was used for the add-back. Rabbit red blood cells (RBCs) or chicken RBCs were washed in GVB/Mg/EGTA, and the depleted serum was spiked with Factor B. The RBC lysis was then monitored with CVF/FD/human Factor B convertase. These were analyzed to create standard curves from which the Factor B lots were compared. FIGS. 5-6 depict a bar graph of controls used for the hemolysis assay and the standard curves measured from the tested engineered proteases from the Factor B add-back hemolysis assay, respectively. Table 12 below presents a summary of the data from engineered proteases based on MTSP-1 and uPA. FIG. 6 also shows that the MTSP-1-based engineered protease F97E/K224N/F99L/D217I/C122S/C[17]S/C[19]S is able to inhibit hemolysis. This engineered protease was tested at two concentrations (samples 1 and 2). All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively.

TABLE 12
Factor B Add-Back Hemolysis Data Summary

| | | [Protease]_{high} | Hu fB Add-Back |
|---|---|---|---|
| Scaffold | Mutation String | (nM) | EC₅₀ (nM) |
| MTSP-1 | C122S | 4000 | 480.4 |
| | | 4000 | 304.0 |
| | | 4000 | 916.7 |
| MTSP-1 | | 4000 | 37.3 |
| | | 400 | 32.2 |
| MTSP-1 | I41G/F97D/F99L/C122S | 4000 | 266.9 |
| | | 4000 | 108.6 |
| | | 4000 | 333.9 |
| MTSP-1 | A77aT/F94Y/C122S/D125G | 4000 | 65.9 |
| | | 4000 | 125.3 |
| MTSP-1 | I41A/F97D/F99L/C122S | 4000 | 282.9 |
| MTSP-1 | I41G/F97D/F99L/C122S/Y146D/C[17]S/C[19]S | 4000 | 282.9 |
| | | 4000 | 394.7 |
| MTSP-1 | I41G/F97E/F99L/C122S/C[17]S/C[19]S | 4000 | 362.5 |
| | | 4000 | 357.5 |
| | | 4000 | 292.5 |
| MTSP-1 | I41N/F97D/F99L/C122S/C[17]S/C[19]S | 4000 | 222.8 |
| MTSP-1 | F97L/T98G/F99V/C122S/C[17]S/C[19]S/- | 4000 | 4593.0 |
| | null_D96insD | 4000 | 3493.0 |

### Example 5: Peptide Cleavage Assay (k_{cat}/K_{M})

Peptide cleavage assays were performed to evaluate k_{cat}/Kₘ of various engineered proteases of the disclosure based on MTSP-1, uPA, or chymase. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively. Cleavage of various substrates was evaluated to determine k_{cat}/Kₘ for protease activity. Briefly, k_{cat}/Kₘ values were determined from the slopes of the linear portion of a plot of Vₒ as a function of substrate concentration. Generally, the following conditions were used: a protease concentration of 50 nM, a substrate concentration of a maximum of 20 μM, and 1.5-fold serial dilutions, and a temperature of 30° C. For the calculations, a semi-automated Michaelis Menten Kinetics protocol with quadruplicate measurements was used.

For chymase-based engineered proteases, the following quenched fluorescence (QF) peptide substrates were used: TQ2-KDVFYQMKK-Lys(SFAM)-NH2 (SEQ ID NO: 29), and TQ2-KDVFYQMKK-Lys(SFAM) (SEQ ID NO: 30). For MTSP-1- and uPA-based engineered proteases, the following peptide substrates were used: 5FAM-EQQKRKIVL-K(QXL520)-NH2 (SEQ ID NO: 31), CPQ2-PEQQKR-K(SFAM)-NH2 (SEQ ID NO: 32), TQ2-GEQQKRKIVL-Lys(SFAM)-NH2 (SEQ ID NO: 33), Ac-QQKR-ACC (SEQ ID NO: 34). Table 13A below presents the various substrates used to test for protease activity at specific cleavage site sequences, and Table 13B below presents the EGVDAE QF (SEQ ID NO: 52) substrate k_{cat}/Kₘ (M ⁻¹s⁻¹) based on various proteases that were tested.

TABLE 13A
Peptide Substrates for Various
Cleavage Site Sequences

| Cleavage site | |
|---|---|
| amino acid | |
| sequence | Peptide substrates used |
| EGVD | Ac-EGVD-ACC (SEQ ID NO: 39) |
| (SEQ ID | CPQ2-EGVDAE-K(5FAM)-K-NH2 |
| NO: 35) | (SEQ ID NO: 40) |
| | Biotin-GGGLSSLTETIEGVDAE-K |
| | (TAMARA)-NH2 (SEQ ID NO: 41) |
| | CPQ2-EGVDAE-K(5FAM)-K-PEG8-K |
| | (Biotin)-NH2 (SEQ ID NO: 42) |
| QQKR | CPQ2-PEQQKR-K(5FAM)-NH2 |
| (SEQ ID | (SEQ ID NO: 32) |
| NO: 36) | TQ2-GEQQKRKIVL-Lys(5FAM)-NH2 |
| | (SEQ ID NO: 33) |
| | Ac-QQKR-ACC (SEQ ID NO: 34) |
| | 5FAM-EQQKRKIVL-K(QXL520)-NH2 |
| | (SEQ ID NO: 31) |
| DVFY | TQ2-KDVFYQMKK-Lys(5FAM)-NH2 |
| (SEQ ID | (SEQ ID NO: 29) |
| NO: 37) | TQ2-KDVFYQMKK-Lys(5FAM) |
| | (SEQ ID NO: 30) |
| | Ac-WEHR-ACC (SEQ ID NO: 43) |
| KNQKR | Ac-KNQK-ACC-TFA Salt |
| (SEQ ID | (SEQ ID NO: 44) |
| NO: 38) | TQ2-KNQKRQKQ-Lys(5FAM)-NH2 |
| | (SEQ ID NO: 45) |
| | Ac-KNQK-ACC (SEQ ID NO: 46) |
| | WSHPQFEKKNQKRQKQ-K(5FAM)-NH2 |
| | (SEQ ID NO: 47) |

TABLE 13B
k_{cat}/K_{M} for Chymase-Based Engineered Proteases
Measured with EGVDAE-QF (SEQ ID NO: 52)

| | EGVDAE QF |
|---|---|
| | (SEQ ID NO: 52) |
| Mutation | k_{cat}/K_{M} (M⁻¹s⁻¹) |
| C22S/F173Y/D175N/A226R | 2665 |
| C22S/K40L/F41R/L99N/F173Y/S218L/A226R | 197 |
| C22S/K40L/F41H/L99N/F173Y/S218L/A226R | 190 |
| C22S/K40L/F41K/L99N/F173Y/S218L/A226R | 194 |
| C22S/K40L/F41R/L99N/F173Y/D175N/S218L/A226R | 793 |
| C22S/K40L/F41H/L99N/F173Y/D175N/S218L/A226R | 638 |
| C22S/K40L/F41K/L99N/F173Y/D175N/S218L/A226R | 721 |

The results of the peptide cleavage assays are presented in Tables 14A-14B below.

TABLE 14A
Peptide Cleavage Assay Data for uPA-Based Scaffold

| | | Specific Activity | | | | | | |
|---|---|---|---|---|---|---|---|---|
| | | (μM substrate/ | | | | k_{cat}/K_{M} (M⁻¹s⁻¹) | | |
| | | min/uM enzyme) | k_{cat}/K_{M} (M⁻¹s⁻¹) | | | (GLAR-ACC) | | |
| | Mutation String | QQKR-ACC | (AGR-ACC) | | | (SEQ ID NO: 49) | | |
| Scaffold | (Chymo #'s) | (SEQ ID NO: 48) | n | Average | ±S.D. | n | Average | ±S.D. |
| uPA | C122S | 0.0 | 4 | 9.68E+04 | 1.37E+03 | 8 | 6.86E+03 | 1.60E+03 |
| uPA | H99L/C122S | | | | | 4 | 3.15E+04 | 4.81E+03 |
| uPA | H99P/C122S | | | | | 2 | 2.19E+05 | 8.64E+03 |
| uPA | G18E/R36S/V38D/ | 0.0 | | | | 2 | 1.75E+03 | 6.06E+01 |
| | C122S/V159A | | | | | | | |
| uPA | H99D/C122S | | | | | 2 | 1.31E+03 | 6.45E+01 |
| uPA | H99N/C122S | 0.2 | | | | 1 | 5.38E+03 | |
| uPA | H99C/C122S | 0.5 | | | | | | |
| uPA | C122S/R217V | 0.1 | | | | 1 | 6.81E+03 | |
| uPA | R36H/S37dP/V38D/ | | | | | 2 | 5.49E+02 | 1.42E+01 |
| | C122S/A184T | | | | | | | |
| uPA | V38D/C122S/ | | 1 | 6.95E+03 | | 1 | 4.90E+04 | |
| | T97a_L97bdel | | | | | | | |
| uPA | V38D/A96G/D97E/ | | 1 | 5.27E+03 | | 1 | 3.05E+04 | |
| | A98G/H99V/C122S/ | | | | | | | |
| | T97a_L97bdel | | | | | | | |
| uPA | C122S/V38D/A96N/ | | 1 | 9.16E+03 | | 1 | 3.12E+04 | |
| | D97A/A98G/H99V/ | | | | | | | |
| | T97a_L97bdel | | | | | | | |
| uPA | V38D/C122S/D97L/ | | 1 | 5.77E+03 | | 1 | 2.54E+04 | |
| | A98G/H99V/ | | | | | | | |
| | T97a_L97bdel | | | | | | | |
| uPA | V38D/C122S/A96E/ | | 1 | 1.32E+04 | | 1 | 1.79E+04 | |
| | D97R/A98G/H99V/ | | | | | | | |
| | T97a_L97bdel | | | | | | | |
| uPA | V38D/C122S/A96E/ | | 1 | 5.05E+04 | | 1 | 4.21E+05 | |
| | D97H/A98G/H99L/ | | | | | | | |
| | T97a_L97bdel | | | | | | | |
| uPA | V38D/A96E/D97E/ | | 1 | 1.04E+04 | | 1 | 8.53E+03 | |
| | C122S/A98G/H99A/ | | | | | | | |
| | T97a_L97bdel | | | | | | | |
| uPA | V38D/C122S/A96D/ | | 1 | 1.53E+04 | | 1 | 8.49E+03 | |
| | D97G/A98G/H99A/ | | | | | | | |
| | T97a_L97bdel | | | | | | | |
| uPA | V38D/D97A/C122S/ | | 1 | 7.60E+04 | | 1 | 5.31E+05 | |
| | A98G/H99L/ | | | | | | | |
| | T97a_L97bdel | | | | | | | |
| uPA | V38D/C122S/A96D/ | | 1 | 3.86E+04 | | 1 | 2.52E+05 | |
| | D97W/A98N/H99L/ | | | | | | | |
| | T97a_L97bdel | | | | | | | |
| uPA | V38D/C122S/A96D/ | | 1 | 4.79E+04 | | 1 | 2.08E+05 | |
| | D97T/A98G/H99M/ | | | | | | | |
| | T97a_L97bdel | | | | | | | |

TABLE 14A
Continued: Peptide Cleavage Assay Data for uPA-Based Scaffold

| | | k_{cat}/Kₘ (M⁻¹s⁻¹) | | |
|---|---|---|---|---|
| | | (QQKR-ACC) | | |
| | Mutation String | (SEQ ID NO: 48) | | |
| Scaffold | (Chymo #'s) | n | Average | ±S.D. |
| uPA | C122S | 5 | 1.67E+02 | 3.05E+01 |
| uPA | H99L/C122S | 2 | 3.24E+03 | 7.27E+01 |
| uPA | H99P/C122S | 2 | 6.18E+03 | 3.05E+02 |
| uPA | G18E/R36S/V38D/C122S/V159A | 0 | | |
| uPA | H99D/C122S | 2 | 6.00E+02 | 2.83E+01 |
| uPA | H99N/C122S | 1 | 3.68E+02 | |
| uPA | H99C/C122S | 2 | 2.30E+03 | 4.47E+02 |
| uPA | C122S/R217V | 2 | 8.42E+02 | 2.10E+02 |
| uPA | R36H/S37dP/V38D/C122S/A184T | 0 | | |
| uPA | V38D/A96E/D97G/C122S/Y172L/A98G/H99M/T97a_L97bdel | 1 | 7.50E+03 | |
| uPA | V38D/C122S/T97a_L97bdel | 1 | 9.14E+03 | |
| uPA | V38D/A96G/D97E/A98G/H99V/C122S/T97a_L97bdel | 2 | 6.86E+04 | 8.34E+03 |
| uPA | C122S/V38D/A96N/D97A/A98G/H99V/T97a_L97bdel | 2 | 7.61E+04 | 3.87E+03 |
| uPA | V38D/C122S/D97L/A98G/H99V/T97a_L97bdel | 2 | 3.64E+04 | 5.64E+03 |
| uPA | V38D/C122S/A96E/D97R/A98G/H99V/T97a_L97bdel | 2 | 5.51E+04 | 5.48E+03 |
| uPA | V38D/C122S/A96E/D97H/A98G/H99L/T97a_L97bdel | 2 | 1.42E+05 | 3.50E+04 |
| uPA | V38D/A96E/D97E/C122S/A98G/H99A/T97a_L97bdel | 2 | 4.43E+04 | 2.65E+03 |
| uPA | V38D/C122S/A96D/D97G/A98G/H99A/T97a_L97bdel | 2 | 9.24E+04 | 2.03E+03 |
| uPA | V38D/D97A/C122S/A98G/H99L/T97a_L97bdel | 2 | 7.19E+04 | 2.26E+04 |
| uPA | V38D/C122S/A96D/D97W/A98N/H99L/T97a_L97bdel | 2 | 2.05E+05 | 5.39E+03 |
| uPA | V38D/C122S/A96D/D97T/A98G/H99M/T97a_L97bdel | 2 | 7.28E+04 | 1.47E+04 |
| uPA | V38D/D97E/L97bV/A98G/H99V/C122S/A96_H99del | 1 | 1.39E+04 | |
| uPA | V38D/A96G/D97A/H99E/C122S/L97b_H99del | 1 | 6.05E+02 | |
| uPA | V38D/C122S/A96_H99delinsPGVE | 1 | 6.74E+02 | |
| uPA | V38D/C122S/L97b_H99del/D97delinsEG | 1 | 1.39E+04 | |
| uPA | V38D/T97aS/L97bV/A98T/H99E/C122S/A96_H99del | 1 | 1.11E+03 | |
| uPA | V38D/C122S/A96del/A98_H99del/-nulldelinsLK | 1 | 2.77E+04 | |
| uPA | V38D/A96G/D97E/A98G/H99E/C122S/T97a_H99del | 1 | 5.74E+02 | |
| uPA | V38D/C122S/A96del/A98_H99del/-nulldelinsRS | 1 | 2.71E+04 | |
| uPA | V38D/D97E/C122S/-null_H99delinsSPG | 1 | 4.31E+03 | |
| uPA | Y40H/C122S | 0 | | |
| uPA | V38D/A98G/H99A/C122S/T97a_L97bdel | 1 | 5.99E+04 | |
| uPA | V38D/A98G/H99A/C122S/L97bdel | 1 | 3.44E+03 | |
| uPA | V38D/A98G/H99L/C122S/T97a_L97bdel | 1 | 8.81E+04 | |

| | | k_{cat}/Kₘ (M−1s−1) | | |
|---|---|---|---|---|
| | | (QQKR/KI-QF) | | |
| | Mutation String | (SEQ ID NO: 50) | | |
| Scaffold | (Chymo #'s) | n | average | ±S.D. |
| uPA | C122S | 0 | | |
| uPA | H99L/C122S | 0 | | |
| uPA | H99P/C122S | 0 | | |
| uPA | H99D/C122S | 0 | | |
| uPA | V38D/A96E/D97G/C122S/Y172L/A98G/H99M/T97a_L97bdel | 0 | | |
| uPA | V38D/C122S/T97a_L97bdel | 0 | | |
| uPA | C122S/V38D/A96N/D97A/A98G/H99V/T97a_L97bdel | 1 | 5.16E+02 | |
| uPA | V38D/C122S/D97L/A98G/H99V/T97a_L97bdel | 0 | | |
| uPA | V38D/C122S/A96E/D97R/A98G/H99V/T97a_L97bdel | 1 | 4.73E+02 | |
| uPA | V38D/C122S/A96E/D97H/A98G/H99L/T97a_L97bdel | 1 | 1.45E+03 | |
| uPA | V38D/A96E/D97E/C122S/A98G/H99A/T97a_L97bdel | 0 | | |
| uPA | V38D/C122S/A96D/D97G/A98G/H99A/T97a_L97bdel | 1 | 2.77E+02 | |
| uPA | V38D/D97A/C122S/A98G/H99L/T97a_L97bdel | 1 | 6.57E+02 | |
| uPA | V38D/C122S/A96D/D97W/A98N/H99L/T97a_L97bdel | 1 | 3.09E+03 | |
| uPA | V38D/C122S/A96D/D97T/A98G/H99M/T97a_L97bdel | 1 | 9.75E+02 | |
| uPA | V38D/C122S/A96del/A98_H99del/-nulldelinsLK | | | |

TABLE 14B
Peptide Cleavage Assay Data for MTSP-1-Based and Chymase-Based Scaffold

| | | Specific Activity | | | |
|---|---|---|---|---|---|
| | | (uMsubstrate/ | k_{cat}/Kₘ (M⁻¹s⁻¹) | | |
| | | min/uMenzyme) | (QHRR-ACC) | | |
| | Mutation String | QQKR-ACC | (SEQ ID NO: 51) | | |
| Scaffold | (Chymo #'s) | (SEQ ID NO: 48) | n | Average | ±S.D. |
| MTSP | C122S | 2.6 | 1 | 3.39E+03 | |
| MTSP | I41G/F97D/F99L/C122S | | 1 | 1.07E+06 | |
| MTSP | A77aT/F94Y/C122S/D125G | | 1 | 4.08E+04 | |
| MTSP | I41A/F97D/F99L/C122S | | 1 | 5.35E+05 | |
| MTSP | I41S/F97D/F99V/C122S/Y146D/ | | 1 | 1.06E+05 | |
| | C[17]S/C[19]S | | | | |
| MTSP | I41G/F97D/F99L/C122S/Y146D/ | | 1 | 6.87E+05 | |
| | C[17]S/C[19]S | | | | |
| MTSP | I41G/F97E/F99L/C122S/C[17]S/ | | 1 | 7.72E+05 | |
| | C[19]S | | | | |
| MTSP | I41N/F97D/F99L/C122S/C[17]S/ | | 1 | 8.05E+05 | |
| | C[19]S | | | | |
| MTSP | I41A/F97D/F99V/C122S/Y146D/Q175T/ | | 1 | 5.03E+04 | |
| | C[17]S/C[19]S | | | | |
| MTSP | I41G/F97D/F99M/C122S/C[17]S/ | | 1 | 2.41E+05 | |
| | C[19]S | | | | |
| MTSP | F97L/T98G/F99V/C122S/C[17]S/ | | 1 | 2.62E+04 | |
| | C[19]S/-null_D96insD | | | | |

| | | k_{cat}/Kₘ (M⁻¹s⁻¹) | | | k_{cat}/Kₘ (M⁻¹s⁻¹) | | | | | |
|---|---|---|---|---|---|---|---|---|---|---|
| | | (QQKR-ACC) | | | (RQAR-ACC) | | | k_{cat}/Kₘ (M⁻¹s⁻¹) | | |
| | Mutation String | (SEQ ID NO: 48) | | | (SEQ ID NO: 54) | | | (angiotensinogen-QF) | | |
| Scaffold | (Chymo #'s) | n | Average | ±S.D. | n | Average | ±S.D. | n | Average | ±S.D. |
| MTSP | C122S | 9 | 2.28E+04 | 1.28E+04 | 7 | 3.89E+06 | 8.69E+05 | | | |
| MTSP | F97E/K224N/F99L/ | 2 | 1.49E+06 | 4.24E+05 | 1 | 2.31E+07 | | | | |
| | D217I/C122S/C[17]S/ | | | | | | | | | |
| | C[19]S | | | | | | | | | |
| MTSP | C[17]S/C[19]S/ | 2 | 1.15E+06 | 2.05E+05 | 1 | 2.36E+07 | | | | |
| | F97E/F99L/C122S/Q175L/ | | | | | | | | | |
| | Q192M/D217I/K224R | | | | | | | | | |
| MTSP | C[17]S/C[19]S/ | 1 | 2.90E+06 | | 1 | 3.72E+07 | | | | |
| | F97M/F99L/C122S/Q175W/ | | | | | | | | | |
| | D217I/K224A | | | | | | | | | |
| MTSP | I41G/F97D/F99L/C122S | 1 | 1.23E+05 | | 1 | 4.91E+06 | | | | |
| MTSP | A77aT/F94Y/C122S/ | 1 | 1.14E+05 | | 1 | 4.27E+06 | | | | |
| | D125G | | | | | | | | | |
| MTSP | I41A/F97D/F99L/C122S | 1 | 9.48E+04 | | 1 | 3.92E+06 | | | | |
| MTSP | I41S/F97D/F99V/C122S/ | 1 | 5.13E+04 | | 1 | 3.85E+05 | | | | |
| | Y146D/C[17]S/ | | | | | | | | | |
| | C[19]S | | | | | | | | | |
| MTSP | I41G/F97D/F99L/C122S/ | 1 | 5.05E+04 | | 1 | 2.66E+06 | | | | |
| | Y146D/C[17]S/ | | | | | | | | | |
| | C[19]S | | | | | | | | | |
| MTSP | I41G/F97E/F99L/C122S/ | 1 | 1.08E+05 | | 1 | 3.50E+06 | | | | |
| | C[17]S/C[19]S | | | | | | | | | |
| MTSP | I41N/F97D/F99L/C122S/ | 1 | 1.24E+05 | | 1 | 2.39E+06 | | | | |
| | C[17]S/C[19]S | | | | | | | | | |
| MTSP | I41A/F97D/F99V/C122S/ | 1 | 3.79E+04 | | 1 | 1.97E+05 | | | | |
| | Y146D/Q175T/C[17]S/ | | | | | | | | | |
| | C[19]S | | | | | | | | | |
| MTSP | I41G/F97D/F99M/C122S/ | 1 | 3.45E+04 | | 1 | 1.28E+06 | | | | |
| | C[17]S/C[19]S | | | | | | | | | |
| MTSP | F97L/T98G/F99V/C122S/ | 1 | 1.08E+04 | | 1 | 2.17E+04 | | | | |
| | C[17]S/ | | | | | | | | | |
| | C[19]S/-null_D96insD | | | | | | | | | |
| MTSP | C122S/C[17]S/ | 1 | 1.17E+04 | | 3 | 3.38E+05 | 1.47E+04 | | | |
| | C[19]S/-null_T98insA | | | | | | | | | |
| MTSP | C122S/C[17]S/ | 1 | 1.92E+03 | | 3 | 5.27E+05 | 3.26E+05 | | | |
| | C[19]S/-null_T98insAA | | | | | | | | | |
| MTSP | C[17]S/C[19]S/ | 2 | 4.96E+05 | 3.20E+04 | 1 | 1.85E+07 | | | | |
| | I41Q/F97T/F99L/C122S/Y146D/ | | | | | | | | | |
| | Q175W | | | | | | | | | |
| MTSP | C[17]S/C[19]S/ | 2 | 1.19E+05 | 1.19E+05 | 1 | 8.78E+06 | | | | |
| | I41F/F97D/F99I/C122S/Y146D/ | | | | | | | | | |
| | Q175S/D217T | | | | | | | | | |
| MTSP | C[17]S/C[19]S/ | 2 | 1.64E+04 | 8.81E+03 | 1 | 1.11E+05 | | | | |
| | I41R/F97D/F99N/C122S/Y146D/ | | | | | | | | | |
| | D217V/K224Q | | | | | | | | | |
| MTSP | C[17]S/C[19]S/I41R/ | 2 | 1.70E+04 | 8.13E+03 | 1 | 1.76E+05 | | | | |
| | F97E/F99N/C122S/Y146D/ | | | | | | | | | |
| | D217Y/K224M | | | | | | | | | |
| MTSP | C[17]S/C[19]S/ | 2 | 3.61E+04 | 2.62E+04 | 1 | 1.05E+07 | | | | |
| | I41G/F97E/F99L/C122S/R161G/ | | | | | | | | | |
| | D217Y/K224Q | | | | | | | | | |
| MTSP | C[17]S/C[19]S/ | 1 | 2.09E+03 | | 1 | 6.67E+03 | | | | |
| | I41R/F97D/F99H/C122S/Y146D/ | | | | | | | | | |
| | Q175P/Q192M/D217F/K224L | | | | | | | | | |
| MTSP | C[17]S/C[19]S/ | 2 | 1.33E+04 | 1.11E+04 | 1 | 4.45E+04 | | | | |
| | I41R/F97E/F99S/C122S/Y146D/ | | | | | | | | | |
| | Q175R/D217I/K224M | | | | | | | | | |
| MTSP | C[17]S/C[19]S/ | 2 | 5.62E+02 | 1.98E+01 | 1 | 7.04E+04 | | | | |
| | I41K/F97D/F99Y/C122S | | | | | | | | | |
| MTSP | I41K/F97D/F99H/C122S/ | 2 | 5.49E+02 | 1.27E+02 | 1 | 7.71E+03 | | | | |
| | C[17]S/C[19]S | | | | | | | | | |
| MTSP | F94Y/F99L/C122S/G197A/ | 1 | 6.73E+05 | | 1 | 4.58E+06 | | | | |
| | C[17]S/C[19]S | | | | | | | | | |
| MTSP | F99L/C122S/L172M/G197A/ | 1 | 1.72E+05 | | 1 | 9.70E+06 | | | | |
| | C[17]S/C[19]S | | | | | | | | | |
| MTSP | D23Y/L70Q/F99L/K110I/C122S/ | 1 | 4.91E+05 | | 1 | 1.12E+07 | | | | |
| | V183A/A204V/C[17]S/ | | | | | | | | | |
| | C[19]S | | | | | | | | | |
| MTSP | F94Y/C122S/C[17]S/ | 2 | 2.23E+05 | 4.54E+03 | 1 | 7.10E+06 | | | | |
| | C[19]S | | | | | | | | | |
| chymase | C22S | | | | | | | 2 | 1.69E+05 | 1.14E+03 |
| chymase | C22S/L99G | | | | | | | 1 | 1.73E+04 | |
| chymase | C22S/F173L | | | | | | | 1 | 1.29E+05 | |
| chymase | C22S/L99G/F173L | | | | | | | 1 | 1.17E+04 | |

| | | k_{cat}/Kₘ (M⁻¹s⁻¹) | | | k_{cat}/Kₘ (M⁻¹s⁻¹) | | |
|---|---|---|---|---|---|---|---|
| | | (DVFY/QM-QF) | | | (NVFY/QM-QF) | | |
| | Mutation String | (SEQ ID NO: 55) | | | (SEQ ID NO: 56) | | |
| Scaffold | (Chymo #'s) | n | Average | ±S.D. | n | Average | ±S.D. |
| chymase | C22S | 2 | 1.79E+05 | 4.99E+03 | 3 | 1.22E+05 | 5.17E+04 |
| chymase | C22S/L99G | 1 | 4.93E+05 | | 1 | 7.20E+05 | |
| chymase | C22S/F173L | 1 | 1.34E+05 | | 1 | 1.32E+05 | |
| chymase | C22S/L99G/F173L | 1 | 3.59E+05 | | 1 | 7.78E+05 | |

### Example 6: Inhibition Tests for Engineered Proteases

Inhibition tests were carried out using chymase-based engineered proteases. Various serpins, which are capable of inhibiting protease activity, were used to test whether these could be used for adequate inhibition of the tested chymase-based engineered proteases, to enable selection of an engineered protease resistant to inhibition in plasma. A summary of the serpin inhibition tests are provided in Table 15 below. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively.

TABLE 15
Serpin Inhibition Summary

| | Plasma | |
|---|---|---|
| Mutation String | Inhibitor | kₐₚₚ(M⁻¹s⁻¹) |
| C22S | Plasma | 31166 |
| | antichymotrypsin | |
| | (ACT) | |
| C22S/A226R | alpha-2- | 812 |
| | marcroglobin | |
| | (A₂M) | |
| | ACT | 1045 |
| C22S/F41I/L99W/G151L/F173K/R217S/A226R | A₂M - | 530-37 |
| | YESD/VM | |
| C22S/F41V/L99W/G151L/F173K/R217S/A226R | A₂M | 329 |
| C22S/L99H/F173Y/K192R/S218L/A226R | A₂M | 356 |
| C22S/L99Y/F173Y/K192R/S218L/A226R | A₂M | 3127 |
| C22S/F173Y/D175N/A226R | A₂M | 645 |
| | ACT | 428 |
| C22S/K40L/F41R/L99N/F173Y/S218L/A226R | plasma ACT | 190 |
| C22S/K40M/F41R/L99N/G151S/F173Y/S218L/A226R | plasma ACT | 317 |
| C22S/K40L/F41H/L99N/F173Y/S218L/A226R | plasma ACT | 193 |
| C22S/K40L/F41L/L99N/G151H/F173Y/S218V/A226R | plasma ACT | 252 |
| C22S/K40L/F41K/L99H/G151S/F173Y/S218L/A226R | plasma ACT | 139 |
| C22S/K40L/F41K/L99H/F173Y/S218L/A226R | plasma ACT | 147 |
| C22S/K40L/F41K/L99N/F173Y/S218L/A226R | plasma ACT | 197 |
| C22S/A190S/A226R | plasma ACT | 1599 |
| C22S/S189T/A190S/A226R | plasma ACT | 935 |
| C22S/K40L/F41R/L99N/F173Y/D175N/S218L/A226R | plasma ACT | 37 |
| C22S/K40L/F41H/L99N/F173Y/D175N/S218L/A226R | plasma ACT | 40 |
| C22S/K40L/F41K/L99N/F173Y/D175N/S218L/A226R | plasma ACT | 28 |
| C22S/P38Q/V138I/V183I/L199V/A226R/F228V | plasma ACT | 269 |
| C22S/K40M/F41R/L99Q/F173Y/D175R/S218I/A226R | 46 min (disc) | |

### Example 6: Factor B Cleavage and Activity of KLK5 Protease

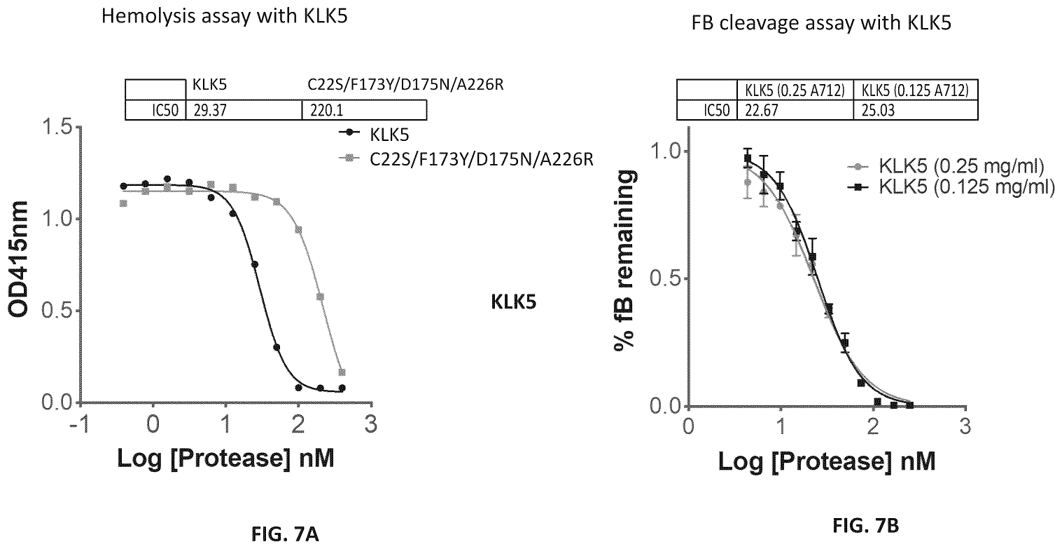

FIG. 7A depicts a graph showing hemolysis inhibition by KLK5 and compared with a chymase-based engineered protease C22S/F173Y/D175N/A226R. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively. Briefly, a digestion reaction was prepared with 4.0 μM of Human Factor B (Complement Technologies) in 20 μL buffer (50 mM Tris pH 7.4/50 mM NaCl/0.01% Tween 20). Different concentration of the KLK5 protease or the chymase-based engineered protease C22S/F173Y/D175N/A226R (400, 200, 100, 50, 25, 12.5, 6.25, 3.125, 1.56, 0.78, and 0.39 with a control at 0.0 nM) were added to 4 μM of Factor B and incubated for 1 hour at 37° C. After digestion, 6 μL of the reaction was evaluated in a hemolysis assay.

The Factor B digestion reactions were diluted in 20 μL of GVB (Complement Technologies), 10 mM MgCl₂ and 8 mM EGTA (GVB/Mg/EGTA). 45 μL of human Factor B Depleted Serum (Complement Technologies) was then added to 5 μL of human Factor B digests to obtain a final volume of 90% serum. In parallel, 50 μL of rabbit red blood cells (Colorado Serum Co.) were diluted into 950 μL of GVB/Mg/EGTA and mixed gently. After spinning at 2000 RPM for 5 minutes at 4° C. the rabbit cells were resuspended in 1 ml of GVB/Mg/EGTA. The washed rabbit cells were then incubated with the mixture of serum+Factor B digests in GVB/Mg/EGTA buffer to obtain a 15% serum final concentration and incubated for 1 hour at 37° C. under agitation. The reaction was then centrifuged at 2000 RPM for 5 minutes and 100 μL of the supernatant was transferred to a clear, flat-bottomed 96 well plate. The plate absorbance was read at 415 nm with a spectrophotometer and the EC₅₀ was calculated. The results of the hemolysis assay are depicted in FIG. 7A, showing that the KLK5 protease was effective in inhibition of hemolysis.

FIG. 7B depicts a graph showing Factor B cleavage with KLK5 and a chymase-based engineered protease C22S/F173Y/D175N/A226R. Briefly, A digestion reaction is prepared with 4.0 uM of Human Factor B (Complement Technologies) in 20 μL buffer (50 mM Tris pH 7.4/50 mM NaCl/0.01% Tween 20). Different concentrations of KLK5 (400, 200, 100, 50, 25, 12.5, 6.25, 3.125, 1.56, 0.78, and 0.39 with a control at 0.0 nM) are added to 4 μM of factor B and incubated for 1 hour at 37° C. After digestion, 15 μL of the reaction is transferred to a 96 well plate with 1.5 μL of 0.2N HCL. After quenching, the reactions are prepared factor B ELISA.

A Factor B standard curve is made with 800, 533.3, 355.5, 237.0, 158.0, 105.3, 70.2, 46.8, 31.2, 20.8, 0.0 pM in 1% BSA-PBST. A 384 well plate was coated with the monoclonal antibody against human Factor Ba (#A225, Quidel) at 2 μg/mL in carbonate buffer (25 μL/well). After blocking for 1 hour at room temperature with 1% BSA-PBST (100 μL/well), the digests (chymase+Factor B) were diluted to 800 pM and standards were diluted 1:1.5 from 800 pM into the blocking buffer (25 μL/well). The plate was agitated for 30 minutes at room temperature.

The biotinylated monoclonal antibody against human Factor Bb (Quidel) was then added at 0.125 μg/ml to detect the bound Factor B. Streptavidin-HRP was then diluted at 1:200 in blocking buffer (25 μL/well) and the plate was agitated for 30 minutes at room temperature. The plate was developed with ELISABright, (50 μL/well) for 1 minute at room temperature and read in the EnVision plate reader. FIG. 7B depicts two independent experiments with different stocks of KLK5. These results show that the KLK5 protease was effective in cleavage Factor B, with comparable activity at the various concentrations used.

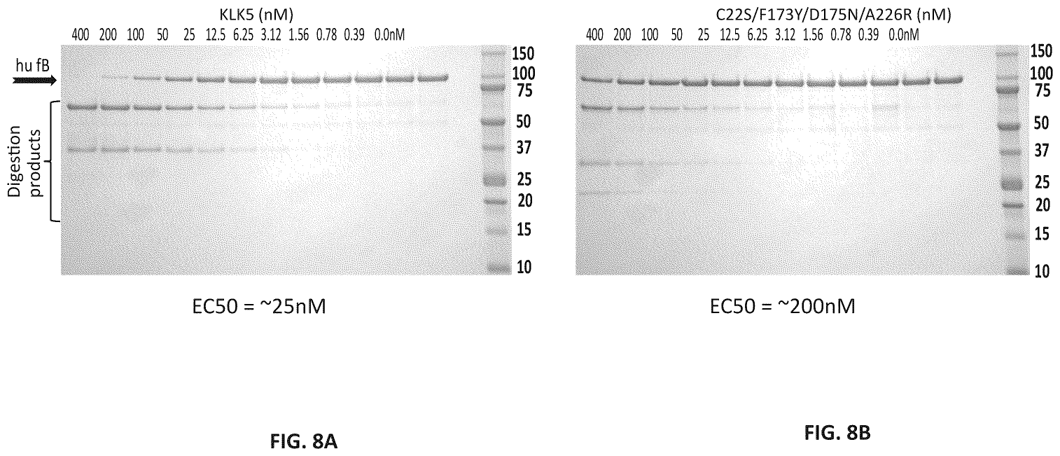

Factor B cleavage by KLK5 was also evaluated by Coomassie gel. FIGS. 8A-8B depict Coomassie gels showing examples of Factor B cleavage with KLK5 and a chymase-based engineered protease C22S/F173Y/D175N/A226R. Briefly, a digestion reaction was prepared with 4.0 μM of Human Factor B (Complement Technologies) in 20 μL buffer (50 mM Tris pH 7.4/50 mM NaCl/0.01% Tween 20). Different concentrations of KLK5 or the chymase-based engineered protease C22S/F173Y/D175N/A226R (400, 200, 100, 50, 25, 12.5, 6.25, 3.125, 1.56, 0.78, and 0.39 with a control at 0.0 nM) were added to 4 μM of Factor B and incubated for 1 hour at 37° C. After digestion, 15 μL of the reaction was transferred to a 96 well plate with 1.5 μL of 0.2N HCL. After quenching, the reactions were prepared for SDS-PAGE gel. 20 μL of the reaction mixtures were loaded per well of a 4-12% Bis-Tris Criterion gel. Densitometry analysis of the Factor B cleavage was performed, and EC₅₀ was calculated.

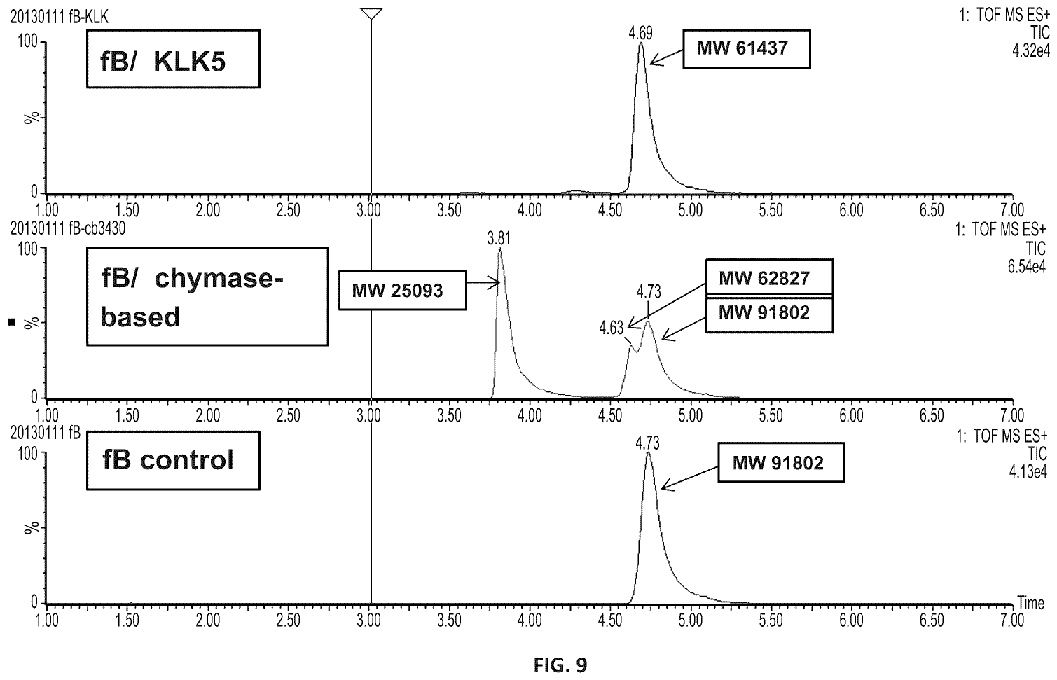

FIG. 9 depicts mass spectrometry (MS) data identifying the cleavage site at 234 Arg within the QQKR/KIV (SEQ ID NO: 9) cleavage site of Factor B by KLK5, and identifying the cleavage site at ²²¹Asp within the EGVDAE (SEQ ID NO: 13) cleavage site of Factor B by the chymase-based engineered protease C22S/A226R. Briefly, human Factor B (Comptech) was incubated at 2 μM with different concentration of Kallikrein 5 (KLK5, R&D Systems) or the chymase-based engineered protease C22S/A226R at 10 nM or 100 pM for 10 minutes to 1 hour at 37° C. in 20 mM Tris pH 8 buffer with 16-0 or 18-0 water. The reaction was quenched with 20 μM of inhibitor FFR-CMK for 30 minutes at room temperature. Half of the sample was further treated with Rapidgest/chymotrypsin and adjusted to pH 3 by the addition of 1 mL 1% TFA. TCEP (100 mM final concentration) was then added to reduce disulfides for 30 minutes at 37° C. 12 mL of each sample was bound to a Ziptip (Millipore) and eluted with 15 mL 80% ACN-0.1% TFA. After drying in a Speedvac, the samples were re-dissolved in 4 ml of 30% ACN-0.05% TFA. 0.35 mL of the sample was loaded with 0.45 mL of CHCA matrix (10 mg/mL) onto an OptiPlate, and analyzed by MALDI-MS (ABI 4700) in both linear (m/z 2-22k) and reflector (m/z 1500-5400) mode. The tandem MS was carried out on peptides of interest when possible. Two large peptide fragments were detected at −33 and 59 kDa. In summary, 100 pM KLK5 cleaves at 1 of 38 Arg in a 10 minute reaction and in 4 out of 38 Arg in a 60 minute reaction (Arg175, Arg193, Arg730 and Arg739). At 10 nM, cleavage was detected in 18 of 38 Arg in 10 and 60 minute reactions (Arg50, Arg74, Arg94, Arg175, Arg182, Arg193, Arg202, Arg203, Arg259, Arg381, Arg415, Arg658, Arg679, Arg708, Arg710, Arg730, Arg739). The earliest cleavage event was at Arg 730.

The reconstructed ion chromatograms shown in FIG. 9 are of reaction products from Kallikrein-5 (“KLK”), the chymase-based engineered protease C22S/A226R (“chymase-based”), and plasma derived-Factor B (“Fb control”). These chromatograms show that the chymase-based engineered protease C22S/A226R cleaves Factor B at ²²¹Asp, wherein the peak at 3.8 minutes corresponding to intact chymase, the peak at 4.73 minutes corresponds to intact Factor B (739 residues with four A2 glycans, MW 91802), and the peak at 4.63 minutes corresponds to cleavage at ²²¹Asp as residues 222-739 containing two A2 glycans (MW 62827). In contrast, KLK5 cleaves Factor B at ²³⁴ Arg (peak at 4.69 minutes, MW 61437). No remaining intact Factor B was present in this sample (top trace).

### Example 7: Complement Activation and Cytokine Release Measured in Mouse Model of Acute Respiratory Distress Syndrome

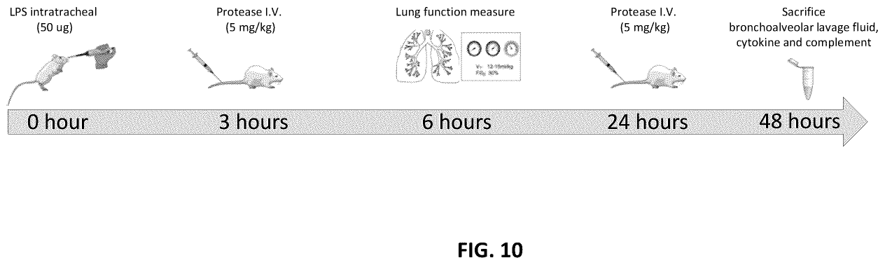

FIG. 10 is a schematic depicting the general method for measuring complement activation and cytokine release from tissues of a mouse model of acute respiratory distress syndrome (ARDS) treated with a chymase-based engineered protease. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively. Briefly, mice received injections of a chymase-based engineered protease C22S/P38Q/K40L/F41R/V138I/F173Y/D175N/A190SN213A/S218V/A226R, and lung function were measured, and post-sacrifice, bronchoalveolar lavage fluid was measured. In FIG. 10, the engineered protease is designated as “protease.”

After an adaptation period, each animal was weighed and randomly assigned to a treatment group based on body weight. On day 0 (0 hours), mice were anaesthetized and received a single intratracheal instillation (IT) of Lipopolysaccharide (LPS, Sigma) at a dosage of 50 μg per mouse. Control mice received an instillation of sterile 0.9% saline (50 μl). All animals were monitored for general health status and body weight over the disease course. Respiratory functions were measured by whole-body plethysmography (WBP) on conscious mice at 0, 6, 24, and 48 hours post-LPS IT. Three (3) hours post-LPS IT, mice received an intravenous (IV) injection of the chymase-based engineered protease C22S/P38Q/K40L/F41R/V138I/F173Y/D175N/A190S/V213A/S218V/A226R at 5 mg/kg or 6.5 mg/kg and control animals received an IV injection of vehicle (PBS). A subset of mice received a second IV injection of the chymase-based engineered protease C22S/P38Q/K40L/F41R/V138I/F173Y/D175N/A190S/V213A/S218V/A226R at 5 mg/kg or 6.5 mg/kg, 27 hours post-LPS IT (i.e. 24 hours post-first IV injection). Mice were sacrificed at 24 and 48 hours post-LPS IT. To obtain plasma, blood was drawn by facial puncture under anesthesia and collected in K₂EDTA microtainer tubes. After centrifuging at 2,000×g for 10 minutes at 4° C. and the plasma was aliquoted (60 μl) and stored at −80° C. for future cytokine and complement analysis.

Next, a tracheotomy was performed to expose the lungs. The trachea was connected to a cannula and the left lung was clamped while 0.9 ml of cold PBS 1X, Protease Inhibitor 1X (SigmaFAST®) solution (3×300 4) was injected to perform a bronchoalveolar lavage fluid (BALF) on the right lobe of the lungs. A first aliquot (300 μL) was kept for BALF total cells count with cells differential count. Two other aliquots of 60 μL each were stored at −80° for future complement and cytokine analysis. The right lung was immediately snap frozen and stored at −80° C. for complement and cytokine analysis in the lung homogenate. The lung was homogenized in 1×PBS+0.1% Triton X-100 with protease cocktail inhibitors to obtain a homogenate of 20 mg/100 μL and centrifuged at 2,520×g for 15 minutes at 4° C. The supernatant was then processed for cytokine analysis.

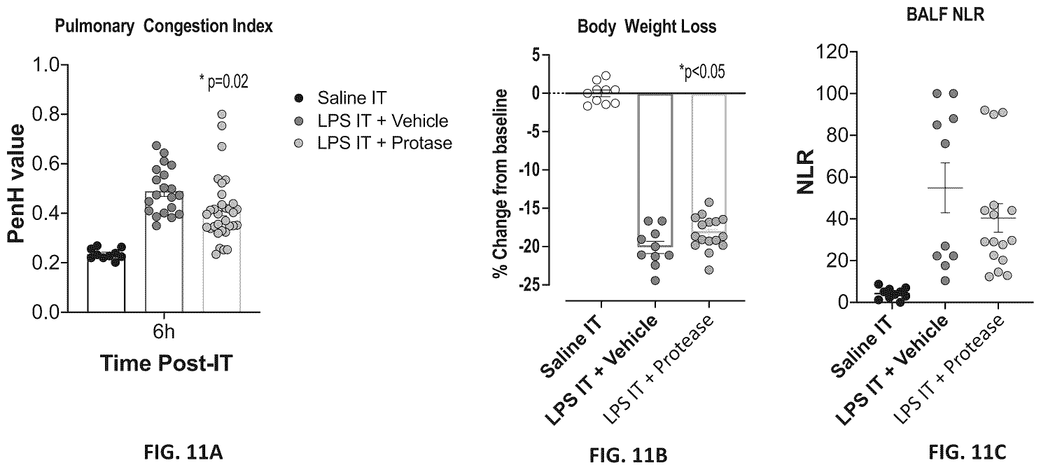

Three hours post injection with the chymase-based engineered protease C22S/P38Q/K40L/F41R/V138I/F173Y/D175N/A190SN213A/S218V/A226R, plethysmography measurements showed a significant reduction in the PenH values suggesting an improvement in lung function. However, this effect is not sustained over time (24 hour and 48 hour measurements). Later, 48 hours post-LPS, animals receiving 2 doses of the chymase-based engineered protease C22S/P38Q/K40L/F41R/V138I/F173Y/D175N/A190SN213A/S218V/A226R at 5-6.5 mg/kg showed a significant improvement of body weight suggesting this engineered protease reduced the severity of ARDS symptoms. At sacrifice 48 hours after LPS, animals receiving 2 doses of the chymase-based engineered protease at 5-6.5 mg/kg showed a trend for a lower neutrophil to lymphocyte ratio in BALF indicating that inflammatory infiltrates are reduced with the chymase-based engineered protease C22S/P38Q/K40L/F41R/V138I/F173Y/D175N/A190S/V213A/S218V/A226R. These results are depicted in FIGS. 11A-11C. FIG. 11A depicts the pulmonary congestion index shown by the PenH value. FIG. 11B depicts body weight loss measured in the tested animals. FIG. 11C depicts the BALF Neutrophil-to-Lymphocyte Ratio (NLR) measured in the tested animals. The data shown are mean+/−SEM, and *p values are <0.05 using the Student's T-test. These results show the chymase-based engineered protease C22S/P38Q/K40L/F41R/V138I/F173Y/D175N/A190S/V213A/S218V/A226R is efficacious in improving respiratory function in a mouse model of ARDS. In FIGS. 11A-11C, this engineered protease is designated as “protease.”

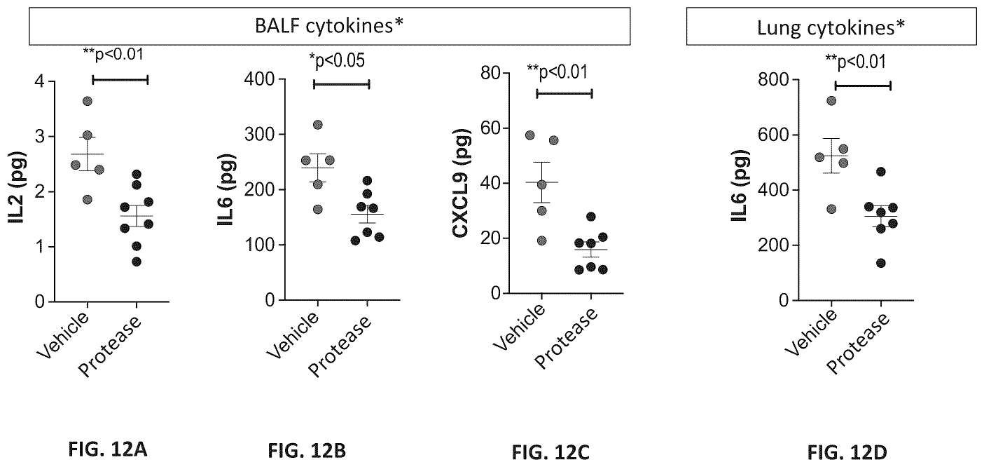

FIGS. 12A-12D depict the results of BALF and lung cytokine measurement from mouse tissue after treatment with a chymase-based engineered protease C22S/P38Q/K40L/F41R/V138I/F173Y/D175N/A190SN213A/S218V/A226R. In FIGS. 12A-12D, this engineered protease is designated as “protease.” Briefly, BALF and lung tissues were collected similarly as described above, 24 hours after a single dose of the chymase-based engineered protease C22S/P38Q/K40L/F41R/V138I/F173Y/D175N/A190S/V213A/S218V/A226R was administered intravenously at 5 mg/kg 3 hours post-LPS. Tissues were processed for quantification of cytokines/chemokines using a Mouse Cytokine Array/Chemokine Array 31-Plex (MD31) measuring Eotaxin, G-CSF, GM-CSF, IFNgamma, IL-1alpha, IL-1beta, IL-2, IL-3, IL-4, IL-5, IL-6, IL-7, IL-9, IL-10, IL-12 (p40), IL-12 (p70), IL-13, IL-15, IL-17A, IP-10, KC, LIF, LIX, MCP-1, M-CSF, MIG, MIP-1alpha, MIP-1beta, MIP-2, RANTES, TNFalpha, and VEGF (Eve Technologies). Samples were centrifuged prior to aliquoting and diluted 2-fold prior to running the assay according to Eve Technology protocol. IL-2, IL-6 and CXCL9 are significantly reduced in mouse bronchoalveolar fluids (BALF) and IL-6 is significantly reduced in lung tissues indicating a lower pulmonary inflammation which may lead to a subsequent reduction in chemoattraction of inflammatory cells into lung tissues. Values are mean+/−SEM and *p<0.05, **p<0.01 using Student's t test. These results show that this engineered protease is efficacious in reducing inflammatory cytokines IL-2 and IL-6, and chemokine CXCL9 in a mouse model of ARDS.

### Example 8: Activity Assessment and Characterization of a Mammalian Expressed Chymase-Based Engineered Protease

The ability for a chymase-based engineered protease zymogen (C22S/P38Q/K40A/F41R/L99H/V138I/F173Y/D175N/A190SN213A/52181/A226R) (Table 7B) (produced in a mammalian culture system, and purified and activated) to cleave CFB was assessed. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively. Enzymatic cleavage of Complement Factor B (CFB, Complement Technologies Cat #A135) was assessed via an AlphaLISA assay. Following in vitro cleavage at 37° C. for 1 hour, reactions were diluted and transferred to a multi-well plate for bead based detection of CFB. For detection of CFB, an anti-Factor Ba antibody (Quidel Cat #A225) labeled with DIG (Biotium Mix N′ Stain Kit, Cat #92450) was paired with anti-DIG acceptor beads (Perkin Elmer Cat #AL113C) and a biotinylated anti Factor-Bb antibody (Quidel Cat #A712) was paired with streptavidin donor beads (Perkin Elmer, Cat #6760002S). If non cleaved CFB remains, the acceptor and donor beads are brought together and a upon laser excitation, a singlet oxygen from the donor bead drives a chemiluminescent signal from the acceptor bead that activates fluorophores contained in the same acceptor bead. Upon CFB cleavage by the engineered protease tested in this example, the resulting Ba and Bb cleavage products are no longer associated and a loss or reduction in signal results. Cleavage of full length CFB was quantified by linear regression against a CFB standard curve in the absence of chymase. Table 16 shows the results of the cleavage of CFB using the listed engineered protease.

TABLE 16
Cleavage of Complement Factor B (CFB)

| Engineered Chymase- | | | |
|---|---|---|---|
| Based Protease | Assay | Metric | Measurement |
| C22S/P38Q/K40A/F41R/ | AlphaLISA | EC₅₀ | 190-212 nM |
| L99H/V138I/F173Y/D175N/ | | | |
| A190S/V213A/S218I/A226R | | | |

The engineered protease was also assessed for inhibition of hemolysis both in a standard Alternative Pathway (AP) hemolysis assay as well as in an enhanced version of the assay, similar to that described in Example 4. Hemolysis inhibition by the small molecule Factor B inhibitor LNP023 (Iptacopan, MedChemExpress Cat #HY-127105) was assessed at in the same experiments as a comparison. For the standard AP assay, varying concentrations of the C22S/P38Q/K40A/F41R/L99HN138I/F173Y/D175N/A190SN213A/S218I/A226R engineered protease or LNP023 or relevant vehicle controls were premixed with 20% Normal Human Serum (NHS) for 10 minutes at 37° C. Rabbit red blood cells (RBCs) were then added along with Alternative Pathway buffer (gelatin veronal buffer, GVB+Mg+EGTA, 0.1% gelatin, 5 mM Veronal, 145 mM NaCl, 0.025% NaN₃, pH 7.3, 10 mM MgCl₂ and 8 mM EGTA) and incubated for 30 minutes at 37° C. Cells were then pelleted and supernatant OD at 415 nm measured to assess lysis. For the enhanced version of the assay the following adjustments were made: NHS was replaced with human Factor B depleted sera (Complement Technologies Cat #A335), Factor B purified from human serum was spiked back into the serum for a final concentration in the hemolysis assay of 1.6 mM, and preincubation of the serum with drug was carried out for 180 min. at 37° C. Percent lysis was calculated using the following formula: [(OD₄₁₅ of sample−OD₄₁₅ EDTA negative control)/(OD₄₁₅ saline positive control-OD₄₁₅ EDTA)*100 ] (Table 17A and Table 17B). IC₅₀s were calculated by nonlinear regression (Prism 9, log(inhibitor,bv) vs. response-4 parameter variable slope model) (Table 18A and Table 18B).

TABLE 17A
Inhibition in Standard AP RBC Hemolysis

| Test | Engineered | | Concentration |
|---|---|---|---|
| Article | Protease | LNP023 | (mM) |
| % Hemolysis | Not tested | 0 | 10 |
| | 3.88 | 0 | 3 |
| | 47.86 | 1.16 | 1 |
| | 88.23 | 0 | 0.33 |
| | 102.20 | 6.08 | 0.11 |
| | Not tested | 93.92 | 0.011 |

TABLE 17B
Inhibition in Enhanced AP RBC Hemolysis

| Test | Engineered | | Concentration |
|---|---|---|---|
| Article | Protease | LNP023 | (mM) |
| % Hemolysis | Not tested | 0 | 10 |
| | 0 | 33.94 | 3 |
| | 4.09 | 108.48 | 1 |
| | 30.45 | 102.50 | 0.33 |
| | 103.94 | 110.53 | 0.11 |
| | 104.70 | Not tested | 0.037 |
| | 112.73 | Not tested | 0.012 |
| | Not tested | 102.27 | 0.011 |
| | 99.24 | Not tested | 0.004 |
| | 104.47 | Not tested | 0.001 |

TABLE 18A
Inhibition in Standard AP RBC Hemolysis

| Test | Engineered | |
|---|---|---|
| Article | Protease | LNP023 |
| IC₅₀ (mM) | 0.92 | 0.027 |

TABLE 18B
Inhibition in Enhanced AP RBC Hemolysis

| Test | Engineered | |
|---|---|---|
| Article | Protease | LNP023 |
| IC₅₀ (mM) | 0.27 | 2.6 |

### Example 9: Lung Function Measured in a Mouse Model of Acute Respiratory Distress Syndrome

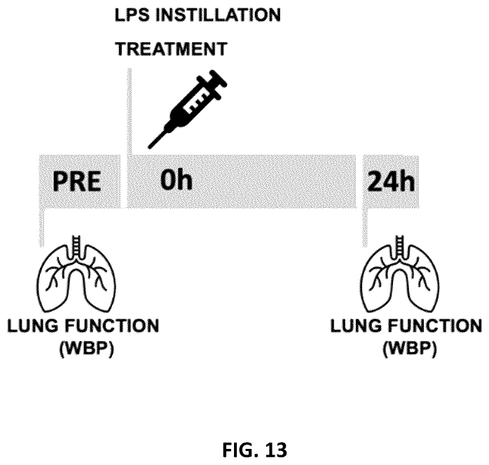

FIG. 13 is a schematic depicting the general method for measuring lung function in a mouse model of acute respiratory distress syndrome (ARDS) treated with a chymase-based engineered protease of the disclosure. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively. Briefly, mice received injections of a chymase-based engineered protease C22S/P38Q/K40A/F41R/L99HN138I/F173Y/D175N/A190SN213A/S218I/A226R or oral gavage of LNP023 (AdooQ Biosciences A18905), and lung function was measured pre-treatment and 24 hours after LPS instillation.

After an adaptation period, each animal was weighed and randomly assigned to a treatment group based on body weight. On day 0 (0 hours), mice were anaesthetized and received a single intratracheal instillation (IT) of Lipopolysaccharide (LPS, Sigma) at a dosage of 50 μg per mouse. All animals were monitored for general health status and body weight over the disease course. Respiratory functions were measured by whole-body plethysmography (WBP) on conscious mice at 0 and 24 hours post-LPS IT. Immediately prior to LPS IT, mice received an intravenous (IV) injection of the chymase-based engineered protease listed above at 5.15 mg/kg. Negative control animals received an IV injection of vehicle (PBS). Active comparator animals received a 30 mg/kg dose of LNP023 administered orally. Mice were sacrificed at 24 post-LPS IT.

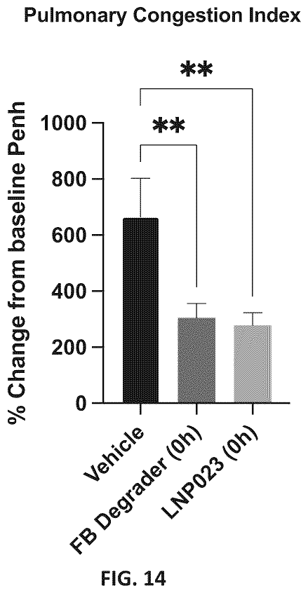

Twenty-four hours post administration of the chymase-based engineered protease listed above or LNP023, plethysmography measurements showed a significant protection to pulmonary congestion, suggesting a protection of lung function with treatment. These results are depicted in FIG. 14. The effects to pulmonary congestion index are shown by the fold-change from baseline of the PenH value. The data shown are mean+/−SEM, and **p values are <0.01 using one-way ANOVA with Dunnett's multiple comparisons test.

Table 19 shows a comparison of the dose levels of the chymase-based engineered protease of the example and LNP023 dose levels administered in vivo

TABLE 19

| | | | | Fold-difference |
|---|---|---|---|---|
| | | Approximate | | molar dose |
| | | Molecular | Molar | (LNP023: |
| | Dose | Weight | Dose | Engineered |
| Treatment | (mg/kg) | (g/mol) | (μmol/kg) | Protease) |
| Engineered | 5.15 | 25100 | 0.2 | ~355 |
| Protease | | | | |
| LNP023 | 30 | 422.5 | 7.1 | |

Table 19 combined with the protective effect of treatment on pulmonary congestion index observed in FIG. 14 demonstrates that the chymase-based engineered protease tested in this example is as efficacious in protecting respiratory function in a mouse model of ARDS as the active comparator LNP023 when administered at approximately a 355-fold lower molar concentration.

The results suggest the efficient regulation of engineered proteases at low concentrations, while small molecule therapeutics require higher concentrations and frequent dosing.

### Example 10: Expression and Purification of Untagged Chymase-Based Engineered Proteases in a Mammalian Expression System

HEK293 cells were transiently transfected with chymase-based engineered protease expression vectors, harvested and clarified by depth filtration. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively. The culture harvest is diluted 1.5 fold with 25 mM Tris HCl, pH7.5 (CCS). The CCS is loaded onto a cation exchange column (Capto SP ImPres or similar) at 20 mL/min. The column is washed with 10 CV of 90% Buffer A (25 mM Tris HCL, pH7.5)+10% Buffer B (with 25 mM Tris HCL, 1M NaCl pH7.5) at 20 mL/min. The recombinant engineered chymase is eluted from the column with a 40 CV linear gradient from 10% Buffer B to 65% Buffer B at 10 mL/min, 5 mL peak fractions containing the engineered chymase are collected, pooled and quantitated by absorbance at 280 nm.

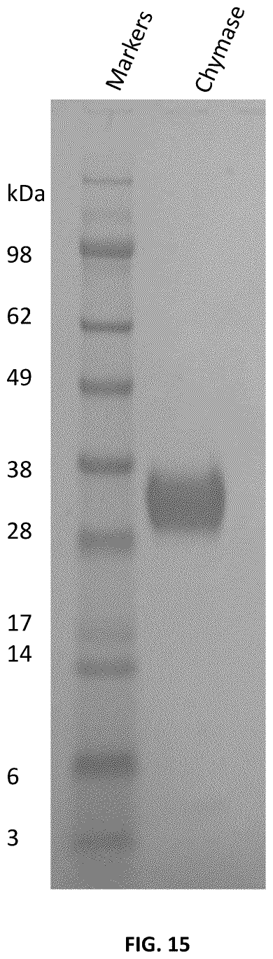

Chymase-based engineered proteases were activated by incubation with enterokinase following adjustment of the pooled fractions to 150 mM NaCl with Buffer A, and addition of CaCl₂) to 4 mM. Activation was initiated with the addition of enterokinase (EKmax, Invitrogen) and incubated at 3TC overnight. Engineered chymase proteases were approximately 90% activated by this method and were further was purified from unactivated chymase and the enterokinase by cation-exchange chromatography using the same procedure outlined above. Pooled fractions are formulated in PBS, 0.1% PS80 (pH 7.4) with purity greater than 98% monomer, HMWS and LMWS less than 2% (FIG. 15).

FIG. 15 is a SDS-PAGE (reduced) depicting the expression, purification, and activation of a chymase-based engineered protease of the disclosure.

### Example 11: Half-Life Extension (HE) and Manufacturability Strategies Using HSA or the IgG1 Fc Domain (Fe)

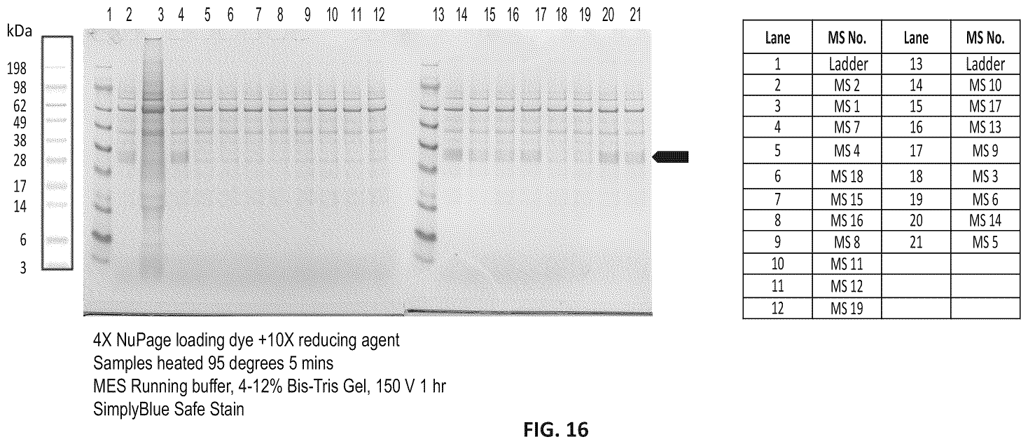

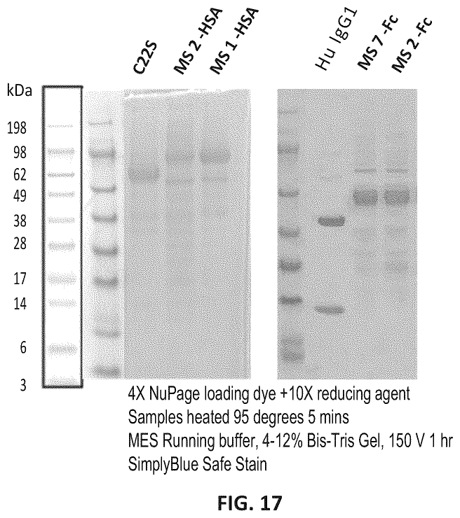

Nineteen chymase-based engineered proteases based on cleavage activity (Table 10) were transiently expression tested as zymogens in HEK293 cells. All references in this Example to engineered proteases are depicted in chymotrypsin numbering. The chymotrypsin numbering key for the modified protease domains of engineered proteases are found in Tables 2, 4, and 6 for uPA, MTSP-1, and chymase, respectively. Eight of the nineteen expressed as evaluated by SDS-PAGE analysis of clarified tissue culture supernatants (Table 20, FIG. 16). Human HSA or Fc were selected as C-terminal fusion partners for selected variants to express transiently in CHO-S or HEK293 cells with the aim of extending solubility, chemical, and in vivo half-life. Both HSA and Fc tagged fusion proteins expressed using the HEK293 transient expression system (FIG. 17). HSA fusion rescued expression for the engineered protease Mutation String Number 1(MS No. 1), which ranked highly based in specific activity in the original E. coli-produced screen.

TABLE 20
Factor B Cleavage Activity of Exemplary Chymase-Based Engineered Proteases

| MS | | EC50 Fb | Expressed |
|---|---|---|---|
| No. | Mutation String | Cleavage | in HEK293 |
| 1 | C22S/P38Q/K40M/F41R/V138I/F173Y/D175R/A190S/V213A/ | 28.1 | |
| | S218V/A226R | | |
| 2 | C22S/P38Q/K40L/F41R/V138I/F173Y/D175N/A190S/V213A/ | 36.3 | Y |
| | S218V/A226R | | |
| 3 | C22S/P38Q/K40M/F41H/V138I/F173Y/D175R/A190S/V213A/ | 47.6 | |
| | S218V/A226R | | |
| 4 | C22S/P38Q/K40M/F41R/V138I/F173L/D175R/A190S/V213A/ | 51.0 | |
| | S218T/A226R | | |
| 5 | C22S/P38Q/K40L/F41R/L99Y/V138I/F173Y/D175N/A190S/ | 57.0 | Y |
| | V213A/S218V/A226R | | |
| 6 | C22S/P38Q/K40L/F41R/L99N/V138I/F173Y/D175T/A190S/ | 57.6 | |
| | V213A/S218V/A226R | | |
| 7 | C22S/P38Q/K40L/F41R/L99H/V138I/F173Y/D175S/A190S/ | 60.3 | Y |
| | V213A/S218I/A226R | | |
| 8 | C22S/K40L/F41K/L99N/F173Y/D175N/S218L/A226R | 61.0 | |
| 9 | C22S/P38Q/K40L/F41R/L99N/V138I/F173Y/D175N/A190S/ | 65.3 | Y |
| | V213A/S218V/A226R | | |
| 10 | C22S/P38Q/K40A/F41R/L99H/V138I/F173Y/D175N/A190S/ | 71.8 | Y |
| | V213A/S218V/A226R | | |
| 11 | C22S/F41K/L99N/F173Y/D175N/S218L/A226R | 77.0 | |
| 12 | C22S/K40L/F41K/F173Y/D175N/S218L/A226R | 77.0 | |
| 13 | C22S/P38Q/K40M/F41R/L99N/V138I/F173Y/D175T/A190S/ | 92.1 | Y |
| | V213A/S218V/A226R | | |
| 14 | C22S/P38Q/K40A/F41R/L99H/V138I/F173Y/D175N/A190S/ | 92.9 | Y |
| | V213A/S218I/A226R | | |
| 15 | C22S/K40L/F41R/L99N/F173Y/D175N/S218L/A226R | 119 | |
| 16 | C22S/K40L/F41H/L99N/F173Y/D175N/S218L/A226R | 139 | |
| 17 | C22S/P38Q/K40A/F41K/L99H/V138I/D175S/A190S/V213A/ | 142.3 | Y |
| | S218V/A226R | | |
| 18 | C22S/F173Y/D175N/A226R | 170 | |
| 19 | C22S/K40L/F41R/L99H/F173Y/D175Y/S218I/A226R | | |

1. An engineered protease comprising a modified chymase protease domain, a modified membrane type serine protease 1 (MTSP-1) protease domain, a modified urokinase-type plasminogen activator (uPA) protease domain, or a modified Kallikrein-related peptidase 5 (KLK5) protease domain, wherein the engineered protease is capable of cleaving Factor B.

2. The engineered protease of claim 1, wherein cleavage of Factor B by the engineered protease generates one or more functionally inactive fragments, and wherein the one or more functionally inactive fragments are capable of reducing complement activation.

3.-4. (canceled)

5. The engineered protease of claim 1, wherein the Factor B is a non-human primate Factor B, or a human Factor B.

6.-8. (canceled)

9. The engineered protease of claim 1, wherein cleavage of Factor B occurs at a site not targeted by Factor D, and/or wherein cleavage of Factor B results in a reduction of the generation of Factor B cleavage products Ba and Bb as compared to cleavage by Factor D.

10.-11. (canceled)

12. The engineered protease of claim 1, wherein cleavage of Factor B occurs at a site that is targeted by Factor D, and wherein the Factor B cleavage site comprises QQKR/KIV (SEQ ID NO: 9).

13. (canceled)

14. The engineered protease of claim 9, wherein the Factor B cleavage site comprises a sequence selected from: WEHR/KGT (SEQ ID NO: 10), KNQKR/QKQ (SEQ ID NO: 11), DVFY/QMI (SEQ ID NO: 12), EGVD/AE (SEQ ID NO: 13), DHKL/KSG (SEQ ID NO: 14), TPW/SLA (SEQ ID NO: 15), KVSEAD (SEQ ID NO: 20), IRPSKG (SEQ ID NO: 4), GGEKRD (SEQ ID NO: 5), GKKEAG (SEQ ID NO: 3), and DHKL/KSG (SEQ ID NO: 21).

15. (canceled)

16. The engineered protease of claim 1, wherein the engineered protease comprises a modified MTSP-1 protease domain.

17. (canceled)

18. The engineered protease of claim 16, comprising one or more modifications with respect to a MTSP-1 protease domain comprising an amino acid sequence as set forth in SEQ ID NO: 7, and wherein the modification is one or more of a substitution, an addition, and deletion of one or more amino acid residues.

19. (canceled)

20. The engineered protease of claim 16, wherein the one or more modifications is at one or more positions selected from D622, 1640, L678, A686, F703, D705, F706, T707, F708, K719, C731, D734, Y755, Q783, V791, Q802, A814, D828, and K835 in a MTSP-1 protease domain comprising the sequence of amino acids set forth in SEQ ID NO: 18; and/or (ii) the one or more modifications are selected from those presented in Table 5A or Table 5B.

21.-22. (canceled)

23. The engineered protease of claim 1, wherein the engineered protease comprises a modified uPA protease domain.

24. (canceled)

25. The engineered protease of claim 23, comprising one or more modifications with respect to a uPA protease domain comprising an amino acid sequence as set forth in SEQ ID NO: 8, and wherein the modification is one or more of a substitution, an addition, and deletion of one or more amino acid residues.

26. (canceled)

27. The engineered protease of claim 23, wherein (i) the one or more modifications is at one or more positions corresponding to one or more positions selected from S37, D60, L97, G161, R179, H180, V185, Y187, 1207, A247, D248, A251, H252, C279, Y308, V316, A343, Q353, R378, K385 in a uPA protease domain comprising the sequence of amino acids set forth SEQ ID NO: 8; and/or (ii) the one or more modifications are selected from those presented in Table 3A or Table 3B.

28.-29. (canceled)

30. The engineered protease of claim 1, wherein the engineered protease comprises a modified chymase protease domain, and the cleavage site comprises a sequence selected from DVFY/QMI (SEQ ID NO: 12), EGVD/AE (SEQ ID NO: 13), DHKL/KSG (SEQ ID NO: 14), and TPW/SLA (SEQ ID NO: 15).

31.-32. (canceled)

33. The engineered protease of claim 30, comprising one or more modifications with respect to a chymase protease domain comprising an amino acid sequence as set forth in SEQ ID NO: 6, and wherein the modification is one or more of a substitution, an addition, and deletion of one or more amino acid residues.

34. (canceled)

35. The engineered protease of claim 30, wherein (i) the one or more modifications is one or more positions corresponding to one or more positions selected from S36 C7, V21, P26, G31, R37, K74, K80, 190, L101, L103, F110, V125, F157, D159, S176, A177, F178, K179, L186, V196, G199, A203, A207, F209 in a chymase protease domain comprising the sequence of amino acids set forth in SEQ ID NO: 6; and/or (ii) the one or more modifications are selected from those presented in Table 7A or Table 7B.

36.-37. (canceled)

38. The engineered protease of claim 1, wherein the engineered protease comprises a modified KLK5 protease domain, optionally comprising one or more amino acid modifications of SEQ ID NO: 23.

39. (canceled)

40. The engineered protease of claim 1, wherein the engineered protease has a k_{cat}/Kₘ of about 100, about 200, about 300, about 400, about 500, about 600, about 700, about 800, about 900, about 1,000, about 1,000, about 1,100, about 1,200, about 1,300, about 1,400, about 1,500, about 1,600, about 1.700, about 1,800, or about 1,900 M⁻¹ s⁻¹ for Factor B cleavage, or wherein the engineered protease has a k_{cat}/Kₘ of about 10³ to about 10⁹ M⁻¹ s⁻¹ for Factor B cleavage.

41. (canceled)

42. The engineered protease of claim 1, wherein the engineered protease has an EC₅₀ for Factor B of about 20, about 25, or about 65 nm, or less than about 20 nM.

43.-45. (canceled)

46. The engineered protease of claim 1, wherein the engineered protease has a plasma half-life in human plasma of over about 72 hours, over about 120 hours, or about 7 days.

47.-48. (canceled)

49. The engineered protease of claim 46, wherein the catalytic activity is about 10% to about 50%, or about 90% to about 100%.

50. The engineered protease of claim 16, wherein the engineered protease has an increased half-life or an increased bioavailability compared to protease comprising a MTSP-1 protease domain that is not modified.

51. (canceled)

52. The engineered protease of claim 23, wherein the engineered protease has an increased half-life or an increased bioavailability compared to a protease comprising a uPA protease domain that is not modified.

53. (canceled)

54. The engineered protease of claim 30, wherein the engineered protease has an increased half-life or an increased bioavailability compared to protease comprising a chymase protease domain that is not modified.

55.-58. (canceled)

59. The engineered protease of claim 1, wherein the engineered protease is fused to a component that extends the half-life of the engineered protease.

60.-61. (canceled)

62. The engineered protease of claim 1, comprising a modified chymase protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 6, and wherein the modified chymase protease domain comprises one of the mutation strings of Table 7B.

63. (canceled)

64. The engineered protease of claim 1, comprising a modified membrane type serine protease 1 (MTSP-1) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 7, and wherein the modified MTSP-1 domain comprises one of the mutation strings of Table 5B.

65. (canceled)

66. The engineered protease of claim 1, comprising a modified urokinase-type plasminogen activator (uPA) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 22, and wherein the modified uPA protease domain comprises one of the mutation strings of Table 3B.

67. (canceled)

68. The engineered protease of claim 1, comprising a modified Kallikrein-related peptidase 5 (KLK5) protease domain having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 95% sequence identity to SEQ ID NO: 23.

69. A method of inactivating Factor B, comprising contacting the Factor B with any of the engineered proteases of claim 1.

70. A method of treating a disease or condition in a subject in need thereof, comprising administering to the subject any one of the engineered proteases of claim 1.

71.-84. (canceled)

85. A pharmaceutical composition comprising any of the engineered proteases of claim 1, and optionally a pharmaceutically acceptable carrier.

86.-87. (canceled)
